(19) United States
(12) Patent Application Publication
(10) Pub. No.: US 2026/0250369 A1
(43) Pub. Date: Aug. 27, 2026

(54) METHODS OF TREATING LIVER FIBROSIS, INFLAMMATION OR ASSOCIATED DISEASES USING AN ANGPTL4 ANTAGONIST

(71) Applicant: Nanyang Technological University, Singapore (SG)

(72) Inventors: Nguan Soon TAN, Singapore (SG); Hong Sheng CHENG, Singapore (SG); Zun Siong LOW, Singapore (SG); Damien CHUA, Singapore (SG)

(21) Appl. No.: 18/872,573

(22) PCT Filed: Jun. 9, 2023
(86) PCT No.: PCT/SG2023/050410

§ 371 Date: Dec. 6, 2024

(30) Foreign Application Priority Data
Jun. 10, 2022 (SG) 10202250125X

(51) Int. Cl. C07K 16/22; A61K 31/713; A61K 39/00; A61P 1/16; A61P 37/02; C12N 15/113

(52) U.S. Cl. CPC C07K 16/22; A61K 31/713; A61P 1/16; A61P 37/02; C12N 15/1136; A61K 2039/505; C07K 2317/24; C07K 2317/30; C07K 2317/76; C12N 2310/14; C12N 2320/30

(57) ABSTRACT

The invention relates generally to methods of treating liver fibrosis and/or inflammation, or associated diseases such as non-alcoholic fatty liver disease (NAFLD) or non-alcoholic steatohepatitis (NASH), using antagonists of angiopoietin like 4 protein (ANGPTL4), more particularly antagonists of ANGPTL4 that are directed against the C-terminal fibrinogen-like domain of ANGPTL4.

### CROSS-REFERENCE TO RELATED APPLICATION

This application claims the benefit of priority of Singapore Patent Application No. 10202250125X filed 10 Jun. 2022, the content of which being hereby incorporated by reference in its entirety for all purposes.

### TECHNICAL FIELD

The present invention generally relates to methods of treating liver fibrosis and/or inflammation, or associated diseases such as non-alcoholic fatty liver disease (NAFLD) or non-alcoholic steatohepatitis (NASH), using antagonists of angiopoietin-like 4 protein (ANGPTL4), more particularly antagonists of ANGPTL4 that are directed against the C-terminal fibrinogen-like domain of ANGPTL4 (cANGPTL4) such that the activity or expression of cANGPTL4 is inhibited.

### BACKGROUND

Non-alcoholic fatty liver disease (NAFLD) is an impending epidemic that affects close to 30% of the world's population (1). NAFLD encompasses all fatty liver disease states, covering a spectrum of liver conditions associated with metabolic dysregulation, starting with liver steatosis, through non-alcoholic steatohepatitis (NASH) that progresses to cirrhosis (2). The global disease prevalence is projected to increase in tandem with the rise of obesity and diabetes mellitus (3). Despite the immense burden of NAFLD, there is still no Food and Drug Administration (FDA)-approved pharmacotherapy. The current standard of care is lifestyle modifications targeting weight loss, which is often difficult to achieve and rarely sustainable (4). Once the disease has progressed to cirrhosis, such measures become less effective (5). Although the challenges and clinical realities have been known for decades, our understanding of the etiology of this multiorgan disease remains sorely lacking, and our ability to treat it remains unacceptably inadequate.

Patients who present with NASH and fibrosis have an increased risk of liver-related mortality (6). As liver fibrosis is the strongest predictor of adverse clinical outcomes, NASH represents the best therapeutic condition, during which most clinical symptoms start to manifest. The reversal of liver fibrosis has the greatest clinical impact in the disease and is a key marker in currently defined endpoints for clinical trials. Chronic hepatic inflammation represents the driving force in the progression of NASH to fibrosis/cirrhosis. The evolution of NAFLD in humans and mice is concomitant with an increase in the prevalence of activated cytotoxic CD8₊ T cells and IFN-γ-producing CD4₊ T cells in the liver. In addition to T cells, B cells are also detectable within the inflammatory infiltrates in liver biopsies from NASH patients. This hepatic infiltration by B and T cells parallels the worsening of liver damage and lobular inflammation (7, 8). Not surprisingly, reduced hepatic recruitment of activated CD4₊ and CD8₊ T cells is observed with decrease in fibrosis (9). Considering the emerging role of adaptive immunity in the progression of NASH, modulating lymphocyte recruitment and activation offers a novel approach to ameliorate NASH-associated fibrosis. To date, the development of therapies for NASH has mainly focused on modulating metabolic dysfunction, oxidative stress, and innate immunity (10). Progress in targeting adaptive immunity in NASH will also rely on a better understanding of the roles of B and T cells in the chronic inflammation process that drives the fibrotic progression in NASH.

Therefore, there is still need in the art to identify novel therapeutic targets for treating Non-alcoholic fatty liver disease (NAFLD) and/or non-alcoholic steatohepatitis (NASH), and more particularly treating, preventing, delaying or ameliorating liver fibrosis or inflammation, or diseases associated with liver fibrosis or inflammation.

### SUMMARY

In a first aspect, there is provided a method for treating, ameliorating, delaying or preventing liver fibrosis or a disease associated with liver fibrosis in a subject, the method comprising administering to the subject an effective amount of an angiopoietin-like 4 protein (ANGPTL4) antagonist.

In various embodiments, the disease associated with liver fibrosis is selected from non-alcoholic steatohepatitis (NASH), autoimmune hepatitis, congenital liver fibrosis, non-alcoholic fatty liver disease (NAFLD), cholestatic liver disease, alcoholic hepatitis, and viral hepatitis.

In various embodiments, the disease associated with liver fibrosis is non-alcoholic steatohepatitis (NASH) or wherein the liver fibrosis is associated with non-alcoholic steatohepatitis (NASH).

In various embodiments, the disease associated with liver fibrosis is non-alcoholic fatty liver disease (NAFLD) or wherein the liver fibrosis is associated with non-alcoholic fatty liver disease (NAFLD).

In various embodiments, the ANGPTL4 antagonist is an antisense molecule; or an aptamer; or an siRNA molecule; or an anti-ANGPTL4 antibody.

In various embodiments, the ANGPTL4 antagonist is directed against the C-terminal fibrinogen-like domain of angiopoietin like 4 (cANGPTL4).

In various embodiments, the ANGPTL4 antagonist neutralizes, blocks, inhibits, reduces or interferes with the expression and/or activity of the C-terminal fibrinogen-like domain of angiopoietin-like 4 (cANGPTL4).

In various embodiments, the ANGPTL4 antagonist is an anti-cANGPTL4 antibody, preferably a monoclonal antibody, more preferably a neutralizing monoclonal antibody.

In various embodiments, the anti-cANGPTL4 antibody is a monoclonal antibody that binds to the same epitope as mAb 11F6C4.

In various embodiments, the anti-cANGPTL4 antibody is a humanized antibody, preferably a humanized mAb 11F6C4.

In various embodiments, the anti-cANGPTL4 antibody specifically binds to the C-terminal fibrinogen-like domain of ANGPTL4.

In various embodiments, the ANGPTL4 antagonist is a siRNA molecule, preferably an ANGPTL4-siRNA molecule.

In various embodiments, the siRNA molecule targets a mRNA sequence encoding ANGPTL4.

In various embodiments, the subject is a human.

In another aspect, there is provided a method for treating, ameliorating or preventing liver inflammation in a subject suffering from liver fibrosis or a disease associated with liver fibrosis, the method comprising administering an effective amount of an angiopoietin-like 4 protein (ANGPTL4) antagonist.

In another aspect, there is provided a method for regulating an immune response in a subject suffering from liver fibrosis or a disease associated with liver fibrosis, the method comprising administering an effective amount of an angiopoietin-like 4 protein (ANGPTL4) antagonist, wherein the administration induces the number, activity and/or effector functions of a population of regulatory T cells, preferably CD8⁺ T and/or CD4+ T cells, to be reduced in the liver of the subject.

In another aspect, there is provided an in vitro method of reducing fibrosis in a liver sample, comprising: contacting the liver cell or tissue with an angiopoietin-like 4 protein (ANGPTL4) antagonist.

In another aspect, there is provided an in vitro method of reducing liver inflammation in a liver sample, comprising: contacting the liver sample with an angiopoietin-like 4 protein (ANGPTL4) antagonist.

In another aspect, there is provided an in vitro method for regulating an immune response in a liver sample, comprising: contacting the liver sample with an angiopoietin-like 4 protein (ANGPTL4) antagonist, wherein the contacting induces the number, activity and/or effector functions of a population of regulatory T cells or activated T-cells, preferably CD8⁺ T and/or CD4+ T cells, to be reduced in the liver sample.

In various embodiments, the ANGPTL4 antagonist is directed against the C-terminal fibrinogen-like domain of angiopoietin like 4 (cANGPTL4).

### BRIEF DESCRIPTION OF THE DRAWINGS

Various embodiments will be better understood with reference to the detailed description when considered in conjunction with the non-limiting examples and the accompanying drawings.

FIG. 1 shows the physiological and metabolic parameters of control and LIDPAD mice: A. Liver weight normalized to body weight after feeding for 1 to 48 weeks; B. Area under the curve (AUC) of intraperitoneal glucose tolerance test (IGTT) curves for the indicated timepoints; C. Proton density fat fraction (PDFF) images from liver of 4-, 8- and 16-week post-diet intervention control and LIDPAD mice. Increased accumulation of hepatic fat is indicated by PDFF scale. Quantification of liver PDFF from control and LIDPAD mice at all imaging timepoints (right). Excessive lipid accretion was observed during 8-12 weeks of LIDPAD intervention. Subcutaneous fat growth identified between timepoints; D. Representative macroscopic and microscopic images of the livers obtained from LIDPAD mice. Histological sections were stained with hematoxylin and eosin (H&E) to show general liver features, picrosirius red (PSR), and Masson trichrome (MT) to highlight collagen deposition, and oil red O (ORO) to detect the presence of lipids. The scale bar represents 100 μm. Graphs show tabulated cumulative histological SAF scores of the livers from LIDPAD mice at the indicated weeks postfeeding (right). Each row corresponds to one analysed liver. For 1A-B, n=7-10 for each group. Data are expressed as the means±SEMs. ****p<0.0001, ***p<0.001, **p<0.01, *p<0.05 (unpaired t test, ANOVA Welch's t test or ANCOVA test when appropriate, followed by post hoc comparisons). n.s. denotes not significant. For 1C, n=5 for each group, *p<0.05 (Mann-Whitney test following pixel thresholding at 60%).

FIG. 2 shows the metabolic parameters of control and LIDPAD mice: A. Change in weight (left) of mice fed the control diet and LIDPAD and the number of animals (left) measured at each time point for 48 weeks; B. IGTT curves of LIDPAD- and control-fed mice from weeks 1 to 48. n=7-10 per group. Data are expressed as the means±SEMs. ****p<0.0001, ***p<0.001, **p<0.01, *p<0.05 (unpaired t test, ANOVA Welch's t test or ANCOVA test when appropriate, followed by post hoc comparisons).

FIG. 3 shows the physiological parameters of control and LIDPAD mice: A-B. Respiratory exchange ratio (RER) (A) of mice fed the control and LIDPAD diets every hour. Gray and white zones represent dark and light cycles that alternate from 0700 to 1900 daily. Mean RER (B) was calculated to determine any statistical differences between control and LIDPAD mice; C-E. Mouse physiology was recorded when challenged with voluntary exercise. Metabolic rate (C) displayed with gray and white zones representing dark and light cycles. The respiratory exchange ratio (D) of LIDPAD mice remained below 0.8, and the daily energy expenditure (E) of LIDPAD mice remained relatively unchanged after introducing voluntary exercise. Data are expressed as the means±SEMs. ***p<0.001 (unpaired t test).

FIG. 4 shows the histological images of pancreatic islets and kidneys from control and LIDPAD mice: A. Representative H&E images of pancreatic islets; B. Quantification and analysis of islet area using ImageJ for control and LIDPAD mice across weeks 1 to 48. n=12-28 region of interest (≥5 islets/animal). Data are expressed as the median±interquartile range; C. Kidney weight (both kidneys) of control and LIDPAD mice compared at week 48. Data information: n=8 per group. Data are expressed as the means±SEMs, ***p<0.001, **p<0.01, *p<0.05 (Welch's t test). n.s. denotes not significant; D. Representative images of kidneys stained using H&E to show the general morphology. Periodic Acid-Schiff (PAS) staining was used to highlight the basement membrane, and Masson trichrome staining was used to identify collagen deposition.

FIG. 5 shows representative macroscopic and microscopic images of the livers obtained from control and LIDPAD mice: A. Histological sections were stained with hematoxylin and eosin (H&E) to show general liver features, picrosirius red (PSR), and Masson's trichrome (MT) to highlight collagen deposition, and oil red O (ORO) to detect the presence of lipids. The scale bar represents 100 μm. The graphs show the tabulated cumulative histological SAF scores of the livers from control mice at the indicated weeks postfeeding (right). Each row corresponds to one analysed liver; B. Representative histological images of mouse livers fed LIDPAD for 16 weeks, stained with H&E and PSR to indicate the hepatic pathological features. * indicates macrovesicular steatosis; † indicates microvesicular steatosis; ‡ indicates ballooned hepatocytes; § indicates the region of lobular inflammation; ¶ indicates the region of pericellular fibrosis; # indicates periportal fibrosis. The scale bar represents 100 μm.

FIG. 6 shows the hepatic transcriptomic and molecular analysis of control and LIDPAD mice: A. Heatmap of differentially expressed genes (DEGs) across weeks 1 to 48 with corresponding Gene Ontology (GO) biological process terms guided by hierarchical clustering; B. Gene ontology and pathway analysis across multiple time points tracking temporal changes in DEGs, featuring only recurring GO terms that span across at least two time points, to increase the confidence of the data as a true positive; C. Transcriptomic profile of LIDPAD mice plotted with orthologues of human genes associated with NAFLD progression. Percentage of genes associated with each histological trait for patients with NASH. Graphs are arranged with increasing severity (left to right). Transcriptomic-based staging of LIDPAD livers was included as a reference; D. GSEA plotted using FDR and size, showing terms from Reactome, WP, KEGG, Biocarta and GO.

FIG. 7 shows hepatic ALT and serum cytokine levels: A. Blood chemistry readout of control and LIDPAD mice based on a liver function test (LFT) consisting of alanine transaminase (ALT). Data are median±interquartile range; B. Lipid analysis highlighting cholesterol and squalene differences between control and LIDPAD mice; C. Heatmap of the serum cytokine array with colour based on the row values after LIDPAD feeding for 1 to 48 weeks. Data are expressed as log (fold change of mean concentration) between LIDPAD and control mice; D. Representative images of liver biopsies from patients with NASH. Scale bar: 200 μm. For A-C: n=3-7 per group. **p<0.01, *p<0.05 (2-way ANOVA with Sidak's multiple comparison for C and Welch's t test for D). n.s. denotes not significant.

FIG. 8 shows the metabolic effects of Angptl4 neutralizing antibodies on NASH mice: A-B. Body weight (A) and IGTT AUC (B) of mice in different treatment groups. AUC: area under the curve; IGTT, intraperitoneal glucose tolerance test; C-D. Representative immunofluorescence images (C) of livers from the indicated treatment groups after feeding for 8 and 12 weeks. CD45 indicates immune infiltration, while α-SMA indicates fibroblast activation. The scale bar represents 100 μm. Barplots showing the quantification of CD45 and α-SMA fluorescence intensities (D) in the immunofluorescence sections; E. FACS analysis showing the proportion of intrahepatic CD45+ immune cells in the indicated groups; F. Representative liver histological images of immunodeficient NSG mice fed a LIPDAD diet for 52 weeks. Data are expressed as the mean±SD. *p<0.05, ***p<0.001. n=6-9 mice per group.

FIG. 9 shows the effects of Angptl4 deficiency on liver pathology and transcriptomes: A. Representative images of microscopic images of the livers obtained from control, LIDPAD-fed mice, LIDPAD-fed mice treated with neutralizing mAb against cAngptl4 and LIDPAD-fed Angptl4LysM−/− mice after 8 and 12 weeks of feeding. Histological sections were stained with hematoxylin and eosin (H&E) to show general liver features and picrosirius red (PSR) to highlight collagen deposition. The scale bar represents 100 μm. The graphs show the tabulated cumulative histological SAF scores of the livers from the indicated groups at the indicated weeks postfeeding (right). Each row corresponds to one analysed liver; B. Graph showing the hepatic triglyceride concentration normalized to the protein level. Data are expressed as the means±SEMs. ***p<0.001, *p<0.05 (unpaired t test). n=5 for each group; C. Principal component analysis (PCA) of liver transcriptomes across the indicated groups and treatments at 8 and 12 weeks; D. Heatmap of differentially expressed genes (DEGs) at weeks 8 and 12 with corresponding Gene Ontology (GO) biological process terms guided by hierarchical clustering of hepatic transcriptomes of the indicated groups and treatments.

FIG. 10 shows the intrahepatic immune landscape during LIDPAD-induced NAFLD: A. Overall UMAP representation of the total intrahepatic CD45+ immune population (left). Dotplot representing molecular markers used to identify major immune cell types (right); B-C. Relative abundance of total CD45+ immune cell types (B) and T-cell populations (C) in the livers of control mice, LIDPAD-fed mice, LIDPAD-fed mice treated with neutralizing monoclonal antibodies against cAngptl4 (mAb) and LIDPAD-fed Angptl4LysM−/− mice after 8 and 12 weeks of feeding.

FIG. 11 shows intrahepatic B- and T-cell profiles: A. UMAP of adaptive immune subpopulations from B and T cells (left). Dot plot representing molecular markers used to identify specific subpopulations of B- and T cells (right); B. GSEA showing highly enriched gene sets in CD8+ T cells during NASH progression from 8 to 12 weeks of feeding based on the differential transcriptomes from different treatment groups. The normalized enrichment score (NES) is color-coded, where NES>0 (red) and <0 (blue) indicate gene functions that were enriched in up- and downregulated genes, respectively. Dot size denotes the −log₁₀ FDR of representative enrichment tests.

FIG. 12 shows that cAngptl4 mAb suppressed intrahepatic CD4 T-cell activation via eIF2α-mediated translation attenuation: A. Proportion of intrahepatic naïve and activated CD4 T cells in control mice, LIDPAD-fed mice, LIDPAD-fed mice treated with neutralizing monoclonal antibodies against cAngptl4 (mAb), and LIDPAD-fed Angptl4LysM−/− mice after 8 and 12 weeks of feeding; B. Pseudotime analysis ordering the CD4 T cells according to the activation status. Vertical lines indicate the median pseudotime score of each group; C. Percentage of intrahepatic naïve (CD45+Lin−CD3+CD4+CD62L+CD69−) and activated (CD45+Lin−CD3+CD4+CD69+) CD4 T cells from the indicated groups after 12 weeks of feeding based on FACS. Lineage markers (Lin) include CD11b, NK1.1 and Ly6G; D-E. GSEA showing highly enriched gene sets in CD4+ T cells during NASH progression from 8 to 12 weeks of feeding (D) and signature genes involved in the regulation of eIF2α phosphorylation (E) based on the differential transcriptomes of naïve CD4 T cells from different treatment groups. Normalized enrichment score (NES)>0 and <0 indicate the enrichment of the gene set by up- and downregulated genes, respectively. For 12D, the NES is color-coded, and the dot size denotes the −log 10 FDR of representative enrichment tests.

FIG. 13 shows the gating strategy of FACS analysis to identify intrahepatic naïve and activated CD4+ and CD8+ T cells.

FIG. 14 shows cAngptl4 mAb suppressed intrahepatic CD8₊ T-cell activation: A. Proportion of intrahepatic naïve and activated CD8 T cells in control mice, LIDPAD-fed mice, and LIDPAD-fed mice treated with neutralizing monoclonal antibodies against cAngptl4 (mAb) after 8 and 12 weeks of feeding; B. Pseudotime analysis ordering the CD8 T cells according to the activation status. Vertical lines indicate the median pseudotime score of each group; C. Percentage of intrahepatic naïve (CD45+Lin−CD3+CD8+CD62L+CD69−) and activated (CD45+Lin−CD3+CD8+CD69+) CD8 T cells from the indicated groups after 12 weeks of feeding based on FACS. Lineage markers (Lin) include CD11b, NK1.1 and Ly6G; D-E. GSEA showing highly enriched gene sets in CD8+ T cells during NASH progression from 8 to 12 weeks of feeding (D) and signature genes involved in the regulation of eIF2α phosphorylation (E) based on the differential transcriptomes of naïve CD8 T cells from different treatment groups. Normalized enrichment score (NES)>0 and <0 indicate the enrichment of the gene set by up- and downregulated genes, respectively. For 14D, the NES is color-coded, and the dot size denotes the −log₁₀ FDR of representative enrichment tests.

FIG. 15 shows distinct roles for CD4+ and CD8+ T cells in NASH severity and liver fibrosis. GSEA of CD4+ T and CD8+ T cells and gene sets in primarily two processes, namely in eukaryotic translation and immune responses. Normalized enrichment score (NES)>0 and <0 indicate the enrichment of the gene set by up- and downregulated genes, respectively. The FDR is color-coded.

FIG. 16 shows A. Eif2ak4 signaling is specifically activated by cANGPTL4 monoclonal antibodies in naïve CD4+ T cells, whereas the gene signatures of effector CD4+ (i.e., Th1 and Th17) and CD8+ T cells were markedly suppressed in activated T cells, regardless of disease progression; B. cANGPTL4 monoclonal antibodies diminished interferon-γ signaling and antigen processing activities in activated CD4+ T cells, both of which are linked to the immune-modulatory capacity of CD4+ T cells.

FIG. 17 shows the kinomic modulatory pathway of the Angptl4-eIF2α phosphorylation pathway: A-B. Kinase inhibitor screens of CD4₊ T cells from wild-type and Angptl4-KO mice using LPS as a stimulant. The heatmap illustrates the expression levels of eIF2α and target genes of phosphorylatedeIF2α (i.e., Ddit3, Atf4 and Hspa5) relative to DMSO-treated wild-type CD4₊ T cells (A). The kinase inhibitors and their molecular targets (in parenthesis) were separated into three groups with differential activities on phospho-eIF2α signalling and Angptl4 dependency based on the expression profiles using a hierarchical clustering method. The schematic diagram (B) outlines six scenarios to justify the role of different protein kinases in the Angptl4-eIF2α axis. Terms and arrows that are shrunken and greyed out indicate suppression of their activities, while those that are enlarged indicate overactivity. Briefly, Angptl4 deficiency in CD4₊ T cells is associated with upregulation of eIF2α and phospho-eIF2α target genes, suggesting an inhibitory effect of Angptl4 on eIF2α expression and its downstream signalling. For protein kinases that do not regulate the eIF2α pathway, their inhibitors do not modify the expression of eIF2α and the target genes in wild-type and Angptl4-knockout CD4₊ T cells (e.g., CK2, ASK, AMPK, CaMK and CDK4/6). When an inhibitor upregulates eIF2α and the target genes in wild-type but not Angptl4-knockout CD4+ T cells (which reflects de-repression of eIF2α in the absence of Angptl4), the regulatory activity of the corresponding molecular targets on the eIF2α pathway is Angptl4-dependent (e.g., MAPK, PI3K, Src, Syk-Btk, etc.). For kinase inhibitors that further upregulate eIF2α and the target genes regardless of the Angptl4 status, the regulatory activity of molecular targets is Angptl4-independent (e.g., PKD1/2/3, JAK1/2/3, Aurora A/B/C, etc.); C-D. mRNA expression of eIF2α (C) and immunoblots of eIF2α and phospho-eIF2α (D) in wild-type and Angptl4-knockout CD4₊ T cells with and without LPS challenge. For (C), 18s was used as the endogenous reference gene while for (D), β-Tubulin which served as a loading control, was from the same samples; E-F. Knockdown efficiency of eIF2α in Angptl4-knockout mouse splenic lymphocytes using SMARTpool siRNA (E). 18s was used as the endogenous reference gene. Activation status of Cell Tracker-labelled, adoptively transferred (top) and host (bottom) CD4₊ T cells in the livers of LIDPAD-fed mice after 12 weeks of feeding (F). Adoptively transferred cells were treated with either si-scrambled or si-eIF2α at 10 nM. Naïve (CD45₊Lin−CD3+CD4+CD62 L+CD69−) and activated (CD45₊Lin₋CD3₊CD4₊CD69₊) CD4 T cells were determined using FACS; G. Schematic diagram summarizing the Angptl4-eIF2α pathway and its role in T-lymphocyte activation during fibrosis in LIDPAD-induced NAFLD mouse model. Angptl4 stimulates the kinases that inhibit phospho-eIF2α signalling, removing its inhibitory effect on T cell activation, i.e., a reduced phosphoeIF2α signalling is associated with more T cell activation.

### DETAILED DESCRIPTION

The following detailed description refers to, by way of illustration, specific details and embodiments in which the invention may be practised. These embodiments are described in sufficient detail to enable those skilled in the art to practice the invention. Other embodiments may be utilized and logical changes may be made without departing from the scope of the invention. The various embodiments are not necessarily mutually exclusive, as some embodiments can be combined with one or more other embodiments to form new embodiments.

Unless otherwise defined, all technical and scientific terms used herein have the same meaning as commonly understood by one of ordinary skill in the art. The singular terms “a,” “an,” and “the” include plural referents unless context clearly indicates otherwise. Similarly, the word “or” is intended to include “and” unless the context clearly indicates otherwise. The term “comprises” means “includes.” In case of conflict, the present specification, including explanations of terms, will prevail. “About”, as used herein in connection with numerical values refers to the referenced numerical value±10% or ±5%.

The inventors of the present application have discovered a key mediatory role of angiopoietin-like 4 (ANGPTL4) in liver fibrosis and/or inflammation and associated liver diseases such as Nonalcoholic fatty liver disease (NAFLD) and non-alcoholic steatohepatitis (NASH). In particular, the C-terminal fibrinogen-like domain of ANGPTL4 (cANGPTL4) has been identified as playing a key role in the development and severity of liver inflammation and liver fibrosis associated with NAFLD or NASH.

In this regard, cANGPTL4 represents a novel therapeutic inhibitory target in treating, preventing or ameliorating liver fibrosis, liver inflammation or diseases associated with said liver fibrosis and/or inflammation, more particularly liver fibrosis and/or inflammation associated with NASH. As exemplified in the experimental data disclosed herein, the systemic inhibition of cANGPTL4 activity using neutralizing monoclonal antibodies was shown to exert a potent suppressive effect on T-cell activation and functionality via the impairment of global protein translation, especially in CD4+ T cells. Essentially, the inhibition of cANGPTL4 activity was shown to remodel the immune cell subpopulations and attenuate the recruitment of adaptive immune cells within the intrahepatic NASH milieu. cANGPTL4 inhibition modulated the translation initiation pathway in CD4+ T cells, subsequently disrupting the maturation of CD4+ and CD8+ T cells into their effector counterparts, modifying the ratio of activated CD4+ and CD8+ T cells in livers, causing a loss of immune and inflammatory activities from these cellular subpopulations.

Thus, the inhibition of cANGPTL4 is translated into reduced inflammation and immune landscape remodelling that ameliorate liver fibrosis and delay the progression of liver diseases, such as NASH.

Further, cANGPTL4 is shown to shape activation of the intrahepatic T-cell landscape through the modulation of eIF2α signalling during fibrosis. Single-immune cell analysis and hepatic transcriptomics during fibrosis, and kinase inhibitor screening confirmed that ANGPTL4 orchestrates the hyperactivation of intrahepatic adaptive immunity via eIF2α signalling. Consistently, inhibition (e.g. immunoblocking) of cANGPTL4 reduces T-cell overactivation, delaying disease aggravation. Thus, cANGPTL4 is a crucial determinant in shaping intrahepatic adaptive immunity during liver fibrosis.

These findings support the potential clinical use of targeting cANGPTL4 with genetic knockdown/knockout, immunoneutralization or other relevant methods to inhibit the activity of cANGPTL4 for treating, preventing, ameliorating or delaying liver fibrosis, liver inflammation or diseases associated with liver fibrosis. In particular, the inhibition of cANGPTL4 activity represents a novel direction for treating, preventing or ameliorating liver fibrosis and/or liver inflammation associated with NASH, and thus may be important for the treatment or prevention of NAFLD.

Accordingly, there is provided a method for treating, ameliorating, delaying or preventing liver fibrosis or a disease associated with liver fibrosis in a subject, the method comprising administering to the subject an effective amount of an angiopoietin-like 4 protein (ANGPTL4) antagonist.

The term “liver fibrosis”, as used herein refers to a condition in which there is an excessive accumulation of scar tissue (fibrous tissue) in the liver, usually as a result of chronic liver injury or inflammation that interferes with liver function. Scar tissue blocks blood flow to the hepatic hilum through organs and interferes with normal functioning. Damage to the liver parenchyma due to inflammation causes activation of stellate cells, which increases fibrosis through the production of myofibroblasts and obstructs blood flow in circulation. Myofibroblast production accelerates the loss of liver function and can lead to death. Liver fibrosis can be caused by various factors, including chronic viral hepatitis B or C infection, excessive alcohol consumption, non-alcoholic fatty liver disease (NAFLD) or non-alcoholic steatohepatitis (NASH), autoimmune hepatitis, certain medications, genetic disorders, and other less common conditions. The severity of liver fibrosis can vary, ranging from mild scarring that may not cause noticeable symptoms, to extensive scarring that can impair liver function and lead to complications such as cirrhosis, liver failure, and portal hypertension. In various embodiments, the indicators of a disease or condition associated with liver fibrosis include increased levels of one or more of the following molecules: α-smooth muscle actin (α-SMA), connective tissue growth factor (CTGF), Vascular cell adhesion factor 1 (VCAM-1), collagen, Yes-associated protein (YAP), phosphorylated YAP, serum alanine aminotransferase (ALT), serum aspartate aminotransferase (AST) and liver Hydroxyproline. As used herein, “chronic liver injury” refers to damage to the liver caused by infection, exposure to drugs or toxic compounds, alcohol, impurities in food, abnormal accumulation of normal substances in the blood, autoimmune processes, genetic defects, or other factors chronic damage. Chronic liver damage may lead to liver fibrosis and cirrhosis.

As used herein, a “disease associated with liver fibrosis” refers to a class of diseases characterized by chronic liver damage and fibrosis. Exemplary “diseases or conditions associated with liver fibrosis” include autoimmune hepatitis, congenital liver fibrosis, non-alcoholic steatohepatitis (NASH), Metabolic Associated Fatty Liver Disease (MAFLD), non-alcoholic fatty liver disease (NAFLD), cholestatic liver disease, alcoholic Hepatitis or viral hepatitis.

In various embodiments, the disease associated with liver fibrosis is selected from non-alcoholic steatohepatitis (NASH), autoimmune hepatitis, congenital liver fibrosis, Metabolic Associated Fatty Liver Disease (MAFLD), non-alcoholic fatty liver disease (NAFLD), cholestatic liver disease, alcoholic hepatitis, and viral hepatitis.

In various embodiments, the disease associated with liver fibrosis is non-alcoholic fatty liver disease (NAFLD).

As used herein, the terms “non-alcoholic fatty liver disease” and “NAFLD” are used interchangeably to refer to a range of conditions affecting people who drink little to no alcohol characterized, at least in part, by excess fat stored in liver cells (e.g. steatosis) not due to alcohol consumption. NAFLD is s typically associated with risk factors such as obesity, insulin resistance, high blood sugar, high cholesterol, and metabolic syndrome. NAFLD may be characterized by any combination of features including steatosis, fibrosis, enlarged liver, fatigue, abdominal pain, abdominal swelling, enlarged blood vessels, enlarged breasts, enlarged spleen, red palms, and jaundice. NAFLD refers to a spectrum of conditions that may range in severity or degree, from simple steatosis, which is the accumulation of fat in the liver without inflammation or liver damage, to non-alcoholic steatohepatitis (NASH), which is characterized by inflammation and liver cell damage, depending on the progression of the disease in a given subject. NAFLD can be sub-classified as non-alcoholic fatty liver (NAFL). Non-alcoholic fatty liver is a type of NAFLD and is a condition in which fat accumulates in the liver cells. NAFL has minimal risk of progressing to cirrhosis. Simple fatty liver usually does not damage the liver, but is a condition that can be identified by liver biopsy. Simple fatty liver is not associated with any other liver abnormalities such as scarring or inflammation. It is a common finding in patients who are very overweight or have diabetes mellitus. A patient has a fatty liver when the fat makes up at least 5% of the liver. As used herein, the term “steatosis” refers to the accumulation of fat in the cells of the liver.

In various embodiments, the NAFLD is independent of obesity and diabetes.

Recently, the term NAFLD has been proposed to be replaced by Metabolic Associated Fatty Liver Disease (MAFLD) (11). MAFLD is a newer term that reflects the evolving understanding of the disease and highlights the strong association between fatty liver disease and metabolic health. According to the proposed criteria, MAFLD is diagnosed when there is evidence of liver fat accumulation, along with the presence of any one of the following three criteria: overweight/obesity, type 2 diabetes or insulin resistance, and evidence of liver inflammation or damage. MAFLD encompasses a broader spectrum of patients who may have fatty liver disease, including those who may not have been traditionally diagnosed with NAFLD due to lack of alcohol consumption but still have metabolic risk factors. In summary, NAFLD is a condition in which excess fat accumulates in the liver of individuals who consume little to no alcohol, while MAFLD is a newer term that reflects a broader understanding of the disease, highlighting the strong association between fatty liver disease and metabolic health, and includes a wider spectrum of patients who may have fatty liver disease.

In various embodiments, the disease associated with liver fibrosis is Metabolic Associated Fatty Liver Disease (MAFLD). In various embodiments, the disease associated with liver fibrosis is NAFLD and MAFLD.

In various embodiments, the MALFD is lean MAFLD. The term “lean MAFLD” as used herein refers to a subtype or variant of metabolic-associated fatty liver disease (MAFLD) that is characterized by the presence of liver fat accumulation and metabolic risk factors, but occurs in subjects with a normal body mass index (BMI) or lower levels of body fat. Lean MAFLD is typically diagnosed in individuals with a BMI less than 25 kg/m², which is considered within the normal or “lean” range.

In various embodiments, the disease associated with liver fibrosis is non-alcoholic steatohepatitis (NASH). In this regard, the liver fibrosis may be associated with non-alcoholic steatohepatitis (NASH). The term “NASH” as used herein, is a more severe form of NAFLD characterized by lipid accumulation, inflammation, hepatocyte ballooning, and varying degrees of fibrosis in the liver, and is regarded as a major cause of cirrhosis of the liver of unknown cause. NASH is usually first suspected in a subject who is found to have elevations in liver tests that are included in routine blood test panels, such as alanine aminotransferase (ALT) or aspartate aminotransferase (AST). When further evaluation shows no apparent reason for liver disease and when x-rays or imaging studies of the liver show fat, NASH is suspected. The only means of providing a definitive diagnosis of NASH and separating it from simple fatty liver is a liver biopsy. NASH is diagnosed when fat along with inflammation and damage to liver cells is observed from the biopsy. If the tissue shows fat without inflammation and damage, NAFL or NAFLD is diagnosed.

As highlighted above and illustrated in the working examples, cANGPTL4 inhibition was shown to modulate the translation initiation pathway in CD4+ T cells, subsequently disrupting the maturation of CD4+ and CD8+ T cells into their effector counterparts, modifying the ratio of activated CD4+ and CD8+ T cells in livers, causing a loss of immune and inflammatory activities from these cellular subpopulations.

Accordingly, there is also provided a method for treating, preventing, delaying or ameliorating liver inflammation in a subject suffering from liver fibrosis or a disease associated with liver fibrosis, the method comprising administering an effective amount of an angiopoietin-like 4 protein (ANGPTL4) antagonist.

The term “liver inflammation”, as used herein refers to the inflammation of the liver tissue. Chronic liver inflammation occurs when the liver is persistently inflamed for an extended period of time, usually more than six months. Chronic hepatitis can result from ongoing viral infections (such as chronic hepatitis B or C), autoimmune hepatitis, metabolic conditions (such as non-alcoholic fatty liver disease or NASH), or other causes. Chronic liver inflammation can lead to liver fibrosis.

In various embodiments, the liver inflammation is chronic liver inflammation. In various embodiments, the liver inflammation is associated with NAFLD or NASH, such that the subject is suffering from said NAFLD or NASH.

In treating, ameliorating, preventing or delaying liver inflammation according to the methods disclosed herein, one effect that may be seen is the decrease in the number of regulatory T-cell or activated T-cell populations, such as CD4+ or CD8+ T-cells. The methods disclosed herein can thus also be considered as methods of affecting or altering the immune response of a subject to whom the ANGPTL4 antagonist is administered. The subject may have liver inflammation in which the immunomodulation of CD4+ or CD8+ T cells is a desired outcome.

In various embodiments, the method and administration of the ANGPTL4 antagonist reduces the number and/or activity of CD8+ T and/or CD4+ T cells, more specifically activated CD8+ T and/or CD4+ T cells, compared to the subject in the absence of the ANGPTL4 antagonist. In various embodiments, the reduction occurs intrahepatic, that is, within the liver cells or tissue of the subject.

Accordingly, there is also provided a method for regulating an immune response in a subject suffering from liver fibrosis or a disease associated with liver fibrosis, the method comprising administering an effective amount of an angiopoietin-like 4 protein (ANGPTL4) antagonist, wherein the administration induces the number, activity and/or effector functions of a population of regulatory T cells, such as CD8⁺ T and/or CD4+ T cells to be reduced or inhibited. In various embodiments, the reduction or inhibition occurs intrahepatic, that is, within the liver cells or tissue.

In addition, there is also provided a method for reducing T cell activation, preferably CD8⁺ T and/or CD4+ T cell activation, in a subject suffering from liver fibrosis or inflammation or a disease associated with liver fibrosis, the method comprising administering an effective amount of an angiopoietin-like 4 protein (ANGPTL4) antagonist. In various embodiments, the reduction of T cell activation occurs intrahepatic, that is, within the liver cells or tissue.

“ANGPTL4” or “Angptl4” refers to angiopoietin-like 4 polypeptide or protein, along with naturally occurring allelic, secreted, and processed forms thereof. ANGPTL4 is a secreted glycoprotein belonging to a family of nine structurally similar Angptl proteins (12). The action of Angptl4 depends on its proteolytic cleavage, releasing an N-terminal coiled-coil domain (nAngptl4) and a C-terminal fibrinogen-like domain (cAngptl4) (12). nAngptl4, present in the plasma, is involved in lipid metabolism through inhibitory binding to lipoprotein lipase, curbing peripheral triglyceride release (13). cAngptl4 interacts with extracellular matrix proteins and is localized in different tissues (14). It has been implicated in various inflammation-associated diseases, such as tissue injury and cancer (15-17). cANGPTL4 modulates inflammation by enhancing vascular permeability, cytokine secretion, and monocyte differentiation (18-20).

The term “ANGPTL4” is also used to refer to fragments (e.g., subsequences, truncated forms, etc.) of the polypeptide comprising, e.g., N-terminal fragment, Coiled-coil domain, C-terminal fragment, fibrinogen-like domain. The term “ANGPTL4” refers to an angiopoietin like protein 4 from any vertebrate or mammalian source, including, but not limited to, human, bovine, chicken, rodent, mouse, rat, porcine, ovine, primate, monkey, and guinea pig, unless specified otherwise. The term also refers to fragments and variants of native ANGPTL4 that maintain at least one in vivo or in vitro activity of a native ANGPTL4. The term encompasses full-length unprocessed precursor forms of ANGPTL4.

ANGPTL4 is found in both humans and mice. For example, ANGPTL4 from a human is a 406 amino acid protein, while the mouse ANGPTL4 is a 410 amino acid protein. In various embodiments, the ANGPTL4 is human ANGPTL4 or murine ANGPTL4.

ANGPTL4 polypeptides and nucleic acids encoding ANGPTL4 are readily known and available to the skilled person and include, for example, a human ANGPTL4 having an NCBI gene ID of 51129, NCBI transcript Reference Sequence: NM_139314, Uniprot ID of Q9BY76 and Ensembl Gene ID of ENSG00000167772, or a murine ANGPTL4 having a NCBI gene ID of 57875, NCBI transcript Reference Sequence: NM_020581, Uniprot ID of Q9Z1P8 and Ensembl Gene ID of ENSMUSG00000002289. For ease of reference, sequences of both human and murine ANGPTL4 are included in the below Table 1.

TABLE 1
Human and Murine Example Sequences of ANGPTL4

| SEQ | | |
|---|---|---|
| ID NO: | Description | Sequence |
| 1 | Human | MSGAPTAGAALMLCAATAVLLSAQGGPVQSKSPRFASWDEMNVLA |
| | ANGPTL4- | HGLLQLGQGLREHAERTRSQLSALERRLSACGSACQGTEGSTDLP |
| | protein | LAPESRVDPEVLHSLQTQLKAQNSRIQQLFHKVAQQQRHLEKQHLR |
| | organism: | IQHLQSQFGLLDHKHLDHEVAKPARRKRLPEMAQPVDPAHNVSRLH |
| | Homo sapiens | RLPRDCQELFQVGERQSGLFEIQPQGSPPFLVNCKMTSDGGWTVI |
| | (Human) | QRRHDGSVDFNRPWEAYKAGFGDPHGEFWLGLEKVHSITGDRNS |
| | Uniprot ID: | RLAVQLRDWDGNAELLQFSVHLGGEDTAYSLQLTAPVAGQLGATT |
| | Q9BY76 | VPPSGLSVPFSTWDQDHDLRRDKNCAKSLSGGWWFGTCSHSNLN |
| | | GQYFRSIPQQRQKLKKGIFWKTWRGRYYPLQATTMLIQPMAAEAAS |
| 2 | Human | SRLHRLPRDCQELFQVGERQSGLFEIQPQGSPPFLVNCKMTSDGG |
| | cANGPTL4- | WTVIQRRHDGSVDFNRPWEAYKAGFGDPHGEFWLGLEKVHSITGD |
| | Fibrinogen C- | RNSRLAVQLRDWDGNAELLQFSVHLGGEDTAYSLQLTAPVAGQLG |
| | terminal | ATTVPPSGLSVPFSTWDQDHDLRRDKNCAKSLSGGWWFGTCSHS |
| | domain | NLNGQYFRSIPQQRQKLKKGIFWKTWRGRYYPLQATTMLIQPMA |
| | (residues 179- | |
| | 406 of SEQ ID | |
| | NO: 1) | |
| | organism: | |
| | Homo sapiens | |
| | (Human) | |
| 3 | Human | agaagccgagctgagcggatcctcacacgactgtgatccgattctttccagcggcttctgcaac |
| | ANGPTL4- | caagcgggtcttacccccggtcctccgcgtctccagtcctcgcacctggaaccccaacgtcccc |
| | mRNA/cDNA | gagagtccccgaatccccgctcccaggctacctaagaggatgagcggtgctccgacggccgg |
| | organism: | ggcagccctgatgctctgcgccgccaccgccgtgctactgagcgctcagggcggacccgtgc |
| | Homo sapiens | agtccaagtcgccgcgctttgcgtcctgggacgagatgaatgtcctggcgcacggactcctgca |
| | (Human) | gctcggccaggggctgcgcgaacacgcggagcgcacccgcagtcagctgagcgcgctgga |
| | NM_139314 | gcggcgcctgagcgcgtgcgggtccgcctgtcagggaaccgaggggtccaccgacctcccgt |
| | | tagcccctgagagccgggtggaccctgaggtccttcacagcctgcagacacaactcaaggctc |
| | | agaacagcaggatccagcaactcttccacaaggtggcccagcagcagcggcacctggaga |
| | | agcagcacctgcgaattcagcatctgcaaagccagtttggcctcctggaccacaagcacctag |
| | | accatgaggtggccaagcctgcccgaagaaagaggctgcccgagatggcccagccagttga |
| | | cccggctcacaatgtcagccgcctgcaccggctgcccagggattgccaggagctgttccaggtt |
| | | ggggagaggcagagtggactatttgaaatccagcctcaggggtctccgccatttttggtgaactg |
| | | caagatgacctcagatggaggctggacagtaattcagaggcgccacgatggctcagtggactt |
| | | caaccggccctgggaagcctacaaggcggggtttggggatccccacggcgagttctggctgg |
| | | gtctggagaaggtgcatagcatcacgggggaccgcaacagccgcctggccgtgcagctgcg |
| | | ggactgggatggcaacgccgagttgctgcagttctccgtgcacctgggtggcgaggacacggc |
| | | ctatagcctgcagctcactgcacccgtggccggccagctgggcgccaccaccgtcccaccca |
| | | gcggcctctccgtacccttctccacttgggaccaggatcacgacctccgcagggacaagaact |
| | | gcgccaagagcctctctggaggctggtggtttggcacctgcagccattccaacctcaacggcca |
| | | gtacttccgctccatcccacagcagcggcagaagcttaagaagggaatcttctggaagacctg |
| | | gcggggccgctactacccgctgcaggccaccaccatgttgatccagcccatggcagcagagg |
| | | cagcctcctagcgtcctggctgggcctggtcccaggcccacgaaagacggtgactcttggctct |
| | | gcccgaggatgtggccgttccctgcctgggcaggggctccaaggaggggccatctggaaactt |
| | | gtggacagagaagaagaccacgactggagaagccccctttctgagtgcaggggggctgcat |
| | | gcgttgcctcctgagatcgaggctgcaggatatgctcagactctagaggcgtggaccaagggg |
| | | catggagcttcactccttgctggccagggagttggggactcagagggaccacttggggccagc |
| | | cagactggcctcaatggcggactcagtcacattgactgacggggaccagggcttgtgtgggtc |
| | | gagagcgccctcatggtgctggtgctgttgtgtgtaggtcccctggggacacaagcaggcgcca |
| | | atggtatctgggcggagctcacagagttcttggaataaaagcaacctcagaaca |
| 4 | Murine | MRCAPTAGAALVLCAATAGLLSAQGRPAQPEPPRFASWDEMNLLA |
| | ANGPTL4- | HGLLQLGHGLREHVERTRGQLGALERRMAACGNACQGPKGKDAP |
| | protein | FKDSEDRVPEGQTPETLQSLQTQLKAQNSKIQQLFQKVAQQQRYLS |
| | organism: Mus | KQNLRIQNLQSQIDLLAPTHLDNGVDKTSRGKRLPKMTQLIGLTPNA |
| | musculus | THLHRPPRDCQELFQEGERHSGLFQIQPLGSPPFLVNCEMTSDGG |
| | (Mouse) | WTVIQRRLNGSVDFNQSWEAYKDGFGDPQGEFWLGLEKMHSITG |
| | Uniprot ID: | NRGSQLAVQLQDWDGNAKLLQFPIHLGGEDTAYSLQLTEPTANELG |
| | Q9Z1P8 | ATNVSPNGLSLPFSTWDQDHDLRGDLNCAKSLSGGWWFGTCSHS |
| | | NLNGQYFHSIPRQRQERKKGIFWKTWKGRYYPLQATTLLIQPMEAT |
| | | AAS |
| 5 | Murine | THLHRPPRDCQELFQEGERHSGLFQIQPLGSPPFLVNCEMTSDGG |
| | cANGPTL4- | WTVIQRRLNGSVDFNQSWEAYKDGFGDPQGEFWLGLEKMHSITG |
| | Fibrinogen C- | NRGSQLAVQLQDWDGNAKLLQFPIHLGGEDTAYSLQLTEPTANELG |
| | terminal | ATNVSPNGLSLPFSTWDQDHDLRGDLNCAKSLSGGWWFGTCSHS |
| | domain | NLNGQYFHSIPRQRQERKKGIFWKTWKGRYYPLQATTLLIQPME |
| | (residues 183- | |
| | 410 of SEQ ID | |
| | NO: 3) | |
| | organism: Mus | |
| | musculus | |
| | (Mouse) | |
| 6 | Murine | gctttataaagtggggctttaggtgcaaccgtgaaacgcttatgagctacgggctccagatcttctt |
| | ANGPTL4- | ctgcaccagagcaagtctaagtctgagccggctcccccagaactccagctgctgggtcttgaac |
| | mRNA/cDNA | tcctgcgttccggagtcctagcgttgctgcacccaaggccacccccagaatcatgcgctgcgct |
| | organism: Mus | ccgacagcaggcgctgccctggtgctatgcgcggctactgcggggcttttgagcgcgcaaggg |
| | musculus | cgccctgcacagccagagccaccgcgctttgcatcctgggacgagatgaacttgctggctcac |
| | (Mouse) | gggctgctacagctcggccatgggctgcgcgaacacgtggagcgcacccgtgggcagctgg |
| | NM_020581 | gcgcgctggagcgccgcatggctgcctgtggtaacgcttgtcaggggcccaagggaaaagat |
| | | gcacccttcaaagactccgaggatagagtccctgaaggccagactcctgagactctgcagagt |
| | | ttgcagactcagctcaaggctcaaaacagcaagatccagcaattgttccagaaggtggcccag |
| | | cagcagagatacctatcaaagcagaatctgagaatacagaatcttcagagccagatagacct |
| | | cttggcccccacgcacctagacaatggagtagacaagacttcgaggggaaagaggcttccca |
| | | agatgacccagctcattggcttgactcccaacgccacccacttacacaggccgccccgggact |
| | | gccaggaactcttccaagaaggggagcggcacagtggacttttccagatccagcctctggggt |
| | | ctccaccatttttggtcaactgtgagatgacttcagatggaggctggacagtgattcagagacgc |
| | | ctgaacggctctgtggacttcaaccagtcctgggaagcctacaaggatggcttcggagatcccc |
| | | aaggcgagttctggctgggcctggaaaagatgcacagcatcacagggaaccgaggaagcc |
| | | aattggctgtgcagctccaggactgggatggcaatgccaaattgctccaatttcccatccatttgg |
| | | ggggtgaggacacagcctacagcctgcagctcactgagcccacggccaatgagctgggtgc |
| | | caccaatgtttcccccaatggcctttccctgcccttctctacttgggaccaagaccatgacctccgt |
| | | ggggaccttaactgtgccaagagcctctctggtggctggtggtttggtacctgtagccattccaatc |
| | | tcaatggacaatacttccactctatcccacggcaacggcaggagcgtaaaaagggtatcttctg |
| | | gaaaacatggaagggccgctactatcctctgcaggctaccaccctgctgatccagcccatgga |
| | | ggctacagcagcctcttagcctcctcactggagcctggttccaggcctaagaagacagtgacttt |
| | | ggttgtggccctgagatttggccattctctgctgggggcaggagctctaagtagggctatctgcgt |
| | | cttgtggacaaagaagaagcccgtaactggagagactggaggaccccttttccgtgttggggtc |
| | | tgcaagcattgttgtctgaaacagtcagagcaacaggaaacaaatggcccagatccagaaaa |
| | | catgggctcgaggggcactgaatatcacttctcgcctaccagagaagttggggatgcagaggg |
| | | accactacagtccaactagctgggcccttaatggcggactcagtcatattgactgactggagac |
| | | agggtgccaggagccctggatacactcatggtgctgttgtaggtgctgtggatgcacaggtgcta |
| | | actgtggttcccaggcacagctcacagcattcttacaataaaaacaacctcagaacaaaaaaa |
| | | aaaaaaa |

In this regard, the C-terminal fibrinogen-like domain of human ANGPTL4 (cANGPTL4) may comprise or consist of amino acids 186 to 406, more preferably amino acids 179-401 of the amino acid sequence as set forth in SEQ ID NO: 1, wherein the position numbering is in accordance with SEQ ID NO: 1. In particular, the C-terminal fibrinogen-like domain of human ANGPTL4 (cANGPTL4) may comprise or consist of the amino acid sequence as set forth in SEQ ID NO: 2.

Further, the C terminal fibrinogen-like domain of mouse ANGPTL4 (cANGPTL4) may comprise or consist of amino acids 186 to 410, more preferably amino acids 183-405, of the amino acid sequence shown as SEQ ID NO: 4, wherein the position numbering is in accordance with SEQ ID NO: 4. In particular, the C-terminal fibrinogen-like domain of murine ANGPTL4 (cANGPTL4) may comprise or consist of the amino acid sequence as set forth in SEQ ID NO: 5.

Accordingly, the ANGPTL4 antagonists described herein are molecules that are capable of neutralizing, blocking, inhibiting, abrogating, reducing or interfering with ANGPTL4 activities, or its expression including its binding to an ANGPTL4 receptor. For example, an ANGPTL4 antagonist can include anti-ANGPTL4 antibodies and antigen-binding fragments thereof, oligonucleotide molecules, small molecules, receptor molecules and derivatives which bind specifically to ANGPTL4 thereby sequestering its binding to one or more receptors, anti-ANGPTL4 receptor antibodies and ANGPTL4 receptor antagonists such as small molecule inhibitors of the receptor. Other ANGPTL4 antagonists also include antagonist variants of ANGPTL4, antisense molecules (e.g., ANGPTL4-S RNA), RNA aptamers, and ribozymes against ANGPTL4 or its receptor, or conjugates or fusion proteins thereof, that inhibit an ANGPTL4 activity, directly or indirectly. In various embodiments, antagonist ANGPTL4 antibodies are antibodies that inhibit or reduce the activity of ANGPTL4 by binding to a specific subsequence or region of ANGPTL4.

In various embodiments, the ANGPTL4 antagonist is directed against the expression or activity of the C-terminal fibrinogen-like domain of angiopoietin-like 4 (cANGPTL4) that comprises or consists of the amino acid sequence as set forth in SEQ ID NO: 2 or 5.

In various embodiments, the ANGPTL4 antagonist is a small molecule. Small molecule antagonists may be identified and chemically synthesized using known methodology (see, e.g., PCT Publication Nos. WO/2000/00823 and WO/2000/39585). In general, small-molecule antagonists are usually less than about 2000 daltons in size, alternatively less than about 1500, 750, 500, 250 or 200 daltons in size, wherein such organic small molecules that are capable of binding, preferably specifically, to the target polypeptide (i.e in this instance ANGPTL4, or more particularly cANGPTL4). Such small-molecule antagonists may be identified without undue experimentation using well-known techniques. In this regard, it is noted that techniques for screening organic small-molecule libraries for molecules that are capable of binding to a polypeptide target are well-known in the art (see, e.g., PCT Publication Nos. WO/2000/00823 and WO/2000/39585).

in various embodiments, the antagonist of ANGPTL4 is an antibody that is directed against the C-terminal region of angiopoietin-like 4 protein (cANGPTL4), and may be termed as an anti-cANGPTL4 antibody. The anti-cANGPTL4 antibody is an antibody that inhibits or reduces the activity of cANGPTL4 by binding to a specific target subsequence or region of the C-terminal fibrinogen-like domain of ANGPTL4 protein.

The term “antibody”, as used herein, refers to a protein consisting of one or more polypeptide chains substantially encoded by all or part of the known immunoglobulin genes. Known immunoglobulin genes, for example in humans, include the kappa (κ), lambda (λ), and heavy chain genetic loci, which together comprise the multitude of variable region genes, and the constant region genes mu (μ), delta (δ), gamma (γ), epsilon (ε), and alpha (α) which encode the IgM, IgD, IgG (IgG1, IgG2, IgG3, and IgG4), IgE, and IgA (IgA1 and IgA2) isotypes respectively. The term “antibody”, as used herein is meant to include full length antibodies and antibody fragments, and may refer to a natural antibody from any organism, an engineered antibody, or an antibody generated recombinantly for experimental, therapeutic, or other purposes. The antibody fragments or variants referred to herein do however always include the heavy chain and light chain variable regions as disclosed herein. Accordingly, such fragments and variants include the known scFv fragments or scFv antibodies. Antibodies may be formulated as a pharmaceutical composition or medicament. The terms “antibody” and “immunoglobulin” are used interchangeably herein to relate to polypeptides encoded by immunoglobulin genes. By “IgG” as used herein is meant a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3.

An antibody directed against the C-terminal region of angiopoietin-like 4 protein (cANGPTL4), means that the antibody specifically recognizes and binds to cANGPTL4. “Specifically binding” and “specific binding”, as used herein, refer to an antibody that binds to its target, i.e. cANGPTL4, based on recognition of an epitope on the target molecule. The anti-cANGPTL4 antibody is an antibody that binds to cANGPTL4 with sufficient affinity and specificity and will usually not bind to other ANGPTL4 homologues, e.g., ANGPTL3. The anti-cANGPTL4 antibody recognizes and binds to the target molecule cANGPTL4 with a binding affinity that is higher than that for other compounds that may be present. In various embodiments, “specifically binding” may mean that the antibody binds to the target molecule cANGPTL4 with at least about a 10⁶-fold greater affinity, preferably at least about a 10⁷-fold greater affinity, more preferably at least about a 10⁸-fold greater affinity, and most preferably at least about a 10⁹-fold greater affinity than it binds molecules unrelated to the target molecule, such as albumins. Typically, specific binding refers to affinities in the range of about 10⁶-fold to about 10⁹-fold greater than non-specific binding. In some embodiments, specific binding may be characterized by affinities greater than 10⁹-fold over non-specific binding. The binding affinity may be determined by any suitable method. Such methods are known in the art and include, without limitation, surface plasmon resonance and isothermal titration calorimetry. For example, binding affinity may be as measured by competition ELISA or by measurement of Kd with BIACORE™, KINEXA™ or PROTEON™. In various embodiments, the antibody uniquely recognizes and binds to the target cANGPTL4.

As the amino acid sequence of the C-terminal region of angiopoietin-like 4 protein (cANGPTL4) are known (e.g. SEQ ID NO: 2 or 5), the person skilled in the art would have no difficulty developing or identifying antibodies which specifically bind to cANGPTL4 with sufficient affinity and specificity.

In various embodiments, the antibody is a monoclonal antibody, more preferably the anti-cANGPTL4 antibody is an anti-cANGPTL4 monoclonal antibody that specifically binds to the C-terminal region of angiopoietin-like 4 protein, more preferably the anti-cANGPTL4 antibody binds to a region or epitope within the amino acid sequence set forth in SEQ ID NO: 2 or 5.

In various embodiments, the antibody specifically binds to an epitope or region within the C-terminal region of angiopoietin-like 4 protein, wherein the C-terminal region of angiopoietin-like 4 protein comprises or consists of the amino acid sequence as set forth in SEQ ID NO: 2 or 5.

To determine the specific region or epitope on cANGPTL4 to which the antibodies bind, epitope mapping may be performed. The term “epitope”, as used herein, refers to its common biochemical sense of a portion of the target protein/antigen capable of being recognized and specifically bound by a particular antibody. In certain embodiments, epitopes include chemically active surface groupings of molecules such as amino acids, sugar side chains, phosphoryl groups, or sulfonyl groups, and, in certain embodiments, may have specific three-dimensional structural characteristics, and/or specific charge characteristics. An antibody epitope typically includes at least 3, and more usually, at least 5 or 8-10 amino acids in a unique spatial conformation. Methods of epitope determination or “epitope mapping” are well known in the art and may be used in conjunction with the instant disclosure to identify epitopes on cANGPTL4 to which antibodies may bind to with sufficient affinity and specificity. In particular, “epitope mapping”, as used herein, refers to the identification and definition of the epitope recognized by an antibody. Various methods can be used including, but not limited to, synthetic peptides (where the sequence of the protein is known), phage display libraries (see epitope library), protein footprinting (using monoclonal antibody to protect the protein from proteolytic degradation), isolation and characterization of the peptide bound to MHC, or expression cloning. Once an epitope on an antigen is determined, it is then possible to competitively screen antibodies for binding to the same epitope. An approach to achieve this is to conduct competition studies to find antibodies that compete for binding to the antigen. A high throughput process for binning antibodies based upon their cross-competition is described in WO/2003/48731 A1.

The term “monoclonal antibody”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they may be synthesized by hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. The monoclonal antibodies can include “chimeric” antibodies and humanized antibodies.

Monoclonal antibodies may be obtained by any technique that provides for the production of antibody molecules by continuous cell lines in culture. These include, but are not limited to the hybridoma technique of Koehler and Milstein (1975), Nature, 256: 495-7; and U.S. Pat. No. 4,376,110), the human B-cell hybridoma technique (Kosbor, et al. (1983), Immunology Today, 4: 72; Cote, et al. (1983), Proc. Natl. Acad. Sci. USA, 80: 2026-30), and the EBV-hybridoma technique (Cole, et al. (1985), in Monoclonal Antibodies And Cancer Therapy, Alan R. Liss, Inc., New York, pp. 77-96). The preparation of monoclonal antibodies specific for a target compound is also described in Harlow and Lane, eds. (1988) Antibodies—A Laboratory Manual. Cold Spring Harbor Laboratory, Chapter 6. Such antibodies may be of any immunoglobulin class including IgG, IgM, IgE, IgA, IgD and any subclass thereof. The hybridoma producing the mAb may be cultivated in vitro or in vivo. Production of high titers of mAbs in vivo makes this a very effective method of production.

In various embodiments, the anti-cANGPTL4 monoclonal antibody is a neutralizing anti-cANGPTL4 monoclonal antibody, such as mAb 3F4F5 (56, 57). The term “neutralizing antibody” or “antibody that neutralizes” as used herein, refers to an antibody that reduces at least one activity of a polypeptide of cANGPTL4 comprising the epitope to which the antibody specifically binds. In various embodiments, a neutralizing antibody reduces an activity in vitro and/or in vivo.

In various embodiments, the neutralizing monoclonal antibody specifically binds to the same epitope as mAb 3F4F5 in cANGPTL4.

In various embodiments, the antibody s a humanized antibody, more preferably the anti-cANGPTL4 antibody is a humanized anti-cANGPTL4 antibody. In various embodiments, the humanized anti-cANGPTL4 antibody is derived from a monoclonal anti-cANGPTL4 antibody.

For example, International patent publication WO2014/027959 A1, which is incorporated by its entirety herein, discloses a murine monoclonal antibody directed against human cANGPTL4 (termed mAb11F6C4). mAb11F6C4 was found to target an epitope that resides within the C-terminus of human ANGPTL4 (cANGPTL4), and does not affect the mitochondrial activities and glucose regulations of this protein. For ease of reference, the amino acid sequences in relation to the VH and VL domains of mAb 11F6C4, as disclosed in WO2014/027959 A1, are recited below in Table 2.

TABLE 2
VH and VL domains of murine mAb 11F6C4

| SEQ | | |
|---|---|---|
| ID NO | Description | Sequence |
| 15 | mAb11F6C4 | QVQLQESGPGILKPSQTLSLTCSFS |
| | heavy chain | GFSLSTSGMGVGWIRQPSGKGLERL |
| | | AHIWWDDDKYYNPSLKSQLTISKDT |
| | | SRNQVFLKIISVDTADTATYYCARK |
| | | DYGSSYDYRGQGTTVTVS |
| 16 | mAb11F6C4 | DIELTQSPASLAVSLGQRATISCKA |
| | light chain | SQSVDYDGDSYLNRFQQKPGQPPKL |
| | | LIYTASNLESGIPARFSGSGSGTDF |
| | | TLNIHPVEEEDAATYYCQQSNEDPW |
| | | TFGGGTKLEIKR |

International patent publication WO2021/221565 A1, which is incorporated by its entirety herein, discloses a humanized antibody directed against the C-terminal region of angiopoietin-like 4 protein (cANGPTL4), more specifically a humanized version of mAb11F6C4 derived from the murine monoclonal antibody mAb11F6C4 as described in WO 2014/027959 A1. In this regard, the murine mAb11F6C4 was humanized in that it comprised a human framework region (FR) and all 6 CDRs from the murine antibody mAb11F6C4 as described in international patent publication WO 2014/027959 A1. For ease of reference, amino acid sequences in relation to the VH and VL domains of humanized mAb11F6C4, as disclosed in WO2021/221565 A1, are recited below in Table 3.

TABLE 3
VH and VL domains of humanized mAb 11F6C4

| SEQ | | |
|---|---|---|
| ID NO | Description | Sequence |
| 7 | VH domain | QITLKESGPTLVKPTQTLTLTCTFS |
| | ANGH1 | GFSLSTSGMGVGWIRQPPGKALEWL |
| | | AHIWWDDDKYYNPSLKSRLTITKDT |
| | | SKNQVVLTMTNMDPVDTATYYCARK |
| | | DYGSSYDYWGQGTLVTVSS |
| 8 | VL domain | DIVMTQSPDSLAVSLGERATINCKA |
| | ANGK1 | SQSVDYDGDSYLNWYQQKPGQPPKL |
| | | LIYTASNLESGVPDRFSGSGSGTDF |
| | | TLTISSLQAEDVAVYYCQQSNEDPW |
| | | TFGQGTKVEIK |
| 9 | VH domain | QVTLKESGPVLVKPTETLTLTCTVS |
| | ANGH2 | GFSLSTSGMGVGWIRQPPGKALEWL |
| | | AHIWWDDDKYYNPSLKSRLTISKDT |
| | | SKSQVVLTMTNMDPVDTATYYCARK |
| | | DYGSSYDYWGQGTLVTVSS |
| 10 | VL domain | EIVLTQSPATLSLSPGERATLSCKA |
| | ANGK2 | SQSVDYDGDSYLNWYQQKPGQAPRL |
| | | LIYTASNLESGIPARFSGSGSGTDF |
| | | TLTISSLEPEDFAVYYCQQSNEDPW |
| | | TFGQGTKLEIK |
| 11 | VH domain | QVTLKESGPTLVKPTQTLTLTCTFS |
| | ANGH3 | GFSLSTSGMGVGWIRQPPGKALEWL |
| | | AHIWWDDDKYYNPSLKSRLTISKDT |
| | | SKNQVVLTMTNMDPVDTATYYCARK |
| | | DYGSSYDYWGQGTTVTVSS |
| 12 | VL domain | DIVMTQSPLSLPVTPGEPASISCKA |
| | ANGK3 | SQSVDYDGDSYLNWYLQKPGQSPRL |
| | | LIYTASNLESGVPDRFSGSGSGTDF |
| | | TLKISRVEAEDVGVYYCFQSNEDPW |
| | | TFGQGTKLEIK |
| 13 | VH domain | QITLKESGPTLVKPTQTLTLTCTFS |
| | ANGH4 | GFSLSTSGMGVGWIRQPPGKALEWL |
| | | AHIWWDDDKYYNPSLKSRLTITKDT |
| | | SKNQVDLTMTFMDPWDTATYYCAHK |
| | | DYGSSYDYWGQGTLVTVSS |
| 14 | VL domain | DIQMTQSPSSLSASVGDRVTITCKA |
| | ANGK4 | SQSVDYDGDSYLNWYQQKPGKAPNL |
| | | LIYTASNLESGVPSRFSGSGSGTDF |
| | | TLTISSLQPEDFATYYCQQSNEDPW |
| | | TFGEGTKVEIK |

In various embodiments, the monoclonal antibody is derived from the murine monoclonal antibody mAb11F6C4. In various embodiments, the monoclonal antibody specifically binds to the same epitope as the murine mAb11F6C4 in cANGPTL4. In various embodiments, the monoclonal antibody is, or is derived from, the murine monoclonal antibody mAb11F6C4, comprising a heavy chain variable domain comprising or consisting of the amino acid sequence set forth in SEQ ID NO:15 or a variant thereof and a light chain variable domain comprising or consisting of the amino acid sequence set forth in SEQ ID NO:16 or a variant thereof.

Said variants are invariable with respect to the CDR regions as defined in WO2014/027959 A1, i.e. any variation occurs in the framework region. Said variants, in various embodiments, share at least 80%, preferably at least 81%, 82%, 83%, 84%, 85%, 86%, 87%, 88%, 89%, 90%, 91%, 92%, 93%, 94%, 95%, 96%, 97%, 98%, or 99% sequence identity with the amino acid sequences set forth in SEQ ID Nos. 15 and 16. The variations, typically amino acid substitutions or deletions, occur in the framework region and not in the CDRs. Possible are for example short truncations on the C- or N-terminus of the variable domain, typically 1, 2, 3 or 4 amino acids in length and/or single amino acid substitutions.

In various embodiments, the monoclonal antibody is, or is derived from, the humanized monoclonal antibody mAb11F6C4. In various embodiments, the monoclonal antibody specifically binds to the same epitope as the humanized mAb11F6C4 in cANGPTL4.

In various embodiments, the monoclonal antibody is a humanized antibody directed against the C-terminal region of angiopoietin-like 4 protein (cANGPTL4), said humanized antibody comprising a heavy chain variable domain and a light chain variable domain with human framework regions. In various embodiments, the humanized antibody comprises:
(i) a heavy chain variable domain comprising or consisting of the amino acid sequence set forth in SEQ ID NO:7 or a variant thereof and a light chain variable domain comprising or consisting of the amino acid sequence set forth in SEQ ID NO:8 or a variant thereof; or
(ii) a heavy chain variable domain comprising or consisting of the amino acid sequence set forth in SEQ ID NO:9 or a variant thereof and a light chain variable domain comprising or consisting of the amino acid sequence set forth in SEQ ID NO:10 or a variant thereof; or
(iii) a heavy chain variable domain comprising or consisting of the amino acid sequence set forth in SEQ ID NO:11 or a variant thereof and a light chain variable domain comprising or consisting of the amino acid sequence set forth in SEQ ID NO:12 or a variant thereof; or
(iv) a heavy chain variable domain comprising or consisting of the amino acid sequence set forth in SEQ ID NO:13 or a variant thereof and a light chain variable domain comprising or consisting of the amino acid sequence set forth in SEQ ID NO:14 or a variant thereof.

Said variants are invariable with respect to the CDR regions as defined in WO2021/221565 A1, i.e. any variation occurs in the framework region. Said variants, in various embodiments, share at least 80%, preferably at least 81%, 82%, 83%, 84%, 85%, 86%, 87%, 88%, 89%, 90%, 91%, 92%, 93%, 94%, 95%, 96%, 97%, 98%, or 99% sequence identity with the amino acid sequences set forth in SEQ ID Nos. 7-14. The variations, typically amino acid substitutions or deletions, occur in the framework region and not in the CDRs. Possible are for example short truncations on the C- or N-terminus of the variable domain, typically 1, 2, 3 or 4 amino acids in length and/or single amino acid substitutions. In various embodiments, in the variants of the heavy chain variable domain the positions corresponding to positions 2, 10, 11, 12, 15, 16, 19, 23, 24, 43, 46, 68, 72, 77, 78, 83, 84, 86, 87, 89, 90, 99, 114, and 115 in SEQ ID NO:7, i.e. using the positional numbering of SEQ ID NO:7, are invariable (in addition to the CDR regions). In various embodiments, in the variants of the light chain variable domain the positions corresponding to positions 1, 3, 4, 9, 10, 12, 13, 14, 15, 17, 18, 19, 20, 21, 22, 40, 46, 47, 49, 62, 64, 78, 80, 81, 82, 83, 84, 87, 88, 89, 104, 107, and 108 in SEQ ID NO:8, i.e. using the positional numbering of SEQ ID NO:8, are invariable (in addition to the CDR regions).

The term “humanized”, as used herein in relation to an antibody, generally refers to an antibody comprising a human framework region (FR) and one or more complementarity determining regions (CDRs) from a non-human (usually mouse or rat) antibody. The non-human antibody providing the CDRs is called the “donor” and the human immunoglobulin providing the framework is called the “acceptor”. Humanization relies principally on the grafting of donor CDRs onto acceptor (human) VL and VH frameworks (Winter U.S. Pat. No. 5,225,539). This strategy is referred to as “CDR grafting”. “Backmutation” of selected acceptor framework residues to the corresponding donor residues is often required to regain affinity that is lost in the initial grafted construct (U.S. Pat. No. 5,693,762). The humanized antibody optimally also will comprise at least a portion of an immunoglobulin constant region, typically that of a human immunoglobulin, and thus will typically comprise a human Fc region. A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein). Humanization or other methods of reducing the immunogenicity of nonhuman antibody variable regions may include resurfacing methods, as described for example in Roguska et al. (1994, Proc Natl Acad Sci USA 91 969-973). In various embodiments, selection-based methods may be employed to humanize and/or affinity mature antibody variable regions, that is, to increase the affinity of the variable region for its target antigen. Other humanization methods may involve the grafting of only parts of the CDRs, including but not limited to methods described in, Tan et al, 2002, J Immunol 169 1119-1125, De Pascalis et al, 2002, J Immunol 169 3076-3084. Structure-based methods may be employed for humanization and affinity maturation, for example as described in U.S. Pat. No. 7,117,096 and related applications.

In various embodiments, the antibodies can be a variety of structures, including, but not limited to antibody fragments that recognize and specifically bind to cANGPTL4. Antibody fragments include but are not limited to bispecific antibodies, minibodies, domain antibodies, synthetic antibodies, antibody mimetics, chimeric antibodies, antibody fusions (sometimes referred to as “antibody conjugates”), and fragments of each, respectively. Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment, which consists of a single variable region, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (viii) bispecific single chain Fv dimers and (ix) “diabodies” or “triabodies”, multivalent or multispecific fragments constructed by gene fusion. The antibody fragments may be modified. For example, the molecules may be stabilized by the incorporation of disulfide bridges linking the VH and VL domains. Examples of antibody formats and architectures are described in Holliger & Hudson, 2006, Nature Biotechnology 23(9):1126-1136, and Carter 2006, Nature Reviews Immunology 6:343-357 and references cited therein. The antibody fragments may be generated by known techniques For example, such fragments include but are not limited to: the F(ab′)2 fragments that can be produced by pepsin digestion of the antibody molecule and the Fab fragments that can be generated by reducing the disulfide bridges of the F(ab′)2 fragments. The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain cross-linking. Alternatively, the relevant cysteine residues are substituted with another amino acid residue or are deleted so as to prevent cross-linking.

In various embodiments, the antagonist of ANGPTL4 is an aptamer that is directed against the C-terminal region of angiopoietin-like 4 protein (cANGPTL4), and may be termed as an cANGPTL4 aptamer.

The term “aptamer” as used herein refers to nucleic acid molecules characterised by the ability to bind to a target molecule (i.e. cANGPTL4) with high specificity and high affinity and is a non-naturally occurring molecule. Aptamers to cANGPTL4 may be identified and/or produced by the method of Systematic Evolution of Ligands by Exponential enrichment (SELEX™). Aptamers targeting angiopoietin-like 4 (ANGPTL4) with high affinity and specificity obtained by SELEX technology have been previously disclosed (57).

In this regard, aptamers may be DNA or RNA molecules and may be single-stranded or double-stranded. The aptamer may comprise chemically modified nucleic acids, for example in which the sugar and/or phosphate and/or base is chemically modified. Such modifications may improve the stability of the aptamer or make the aptamer more resistant to degradation. Aptamers may be synthesised by methods which are well known to the skilled person. For example, aptamers may be chemically synthesised, e.g. on a solid support. Solid phase synthesis may use phosphoramidite chemistry. Aptamers can be thought of as the nucleic acid equivalent of monoclonal antibodies and often have K_{d}'s in the nM or pM range, e.g. less than one of 500 nM, 100 nM, 50 nM, 10 nM, 1 nM, 500 pM, 100 pM. As with monoclonal antibodies, they may be useful in virtually any situation in which target binding is required, including use in therapeutic and diagnostic applications, in vitro or in vivo. Aptamers may be formulated as a pharmaceutical composition or medicament.

In various embodiments, the antagonist of ANGPTL4 is an oligonucleotide molecule that is directed against the C-terminal region of angiopoietin-like 4 protein (cANGPTL4). In particular, the oligonucleotide molecule represses or inhibits the expression of ANGPTL4, and as a consequence cANGPTL4. Accordingly, in this context, the phrase “directed against the cANGPTL4” refers to the inhibition or interference of the cANGPTL4 protein being expressed and produced via the inhibition or interference of expression of a nucleic acid encoding ANGPTL4.

The term “oligonucleotide molecules” as used herein refers to nucleic acid molecules, particularly RNA, employed to regulate gene expression. These include antisense oligonucleotides, targeted degradation of mRNAs by small interfering RNAs (siRNAs), post transcriptional gene silencing (PTGs), developmental\hat{ } regulated sequence-specific translational repression of mRNA by micro-RNAs (miRNAs) and targeted transcriptional gene silencing. An antisense oligonucleotide is an oligonucleotide, preferably single stranded, that targets and binds, by complementary sequence binding, to a target oligonucleotide, e.g. mRNA. Where the target oligonucleotide is an mRNA, binding of the antisense to the mRNA blocks the translation of the mRNA and expression of the gene product, Antisense oligonucleotides may be designed to bind sense genomic nucleic acid and inhibit transcription of a target nucleotide sequence. In view of the known nucleic acid sequences for ANGPTL4, and cANGPTL4 (e.g. the known mRNA sequences available for human and (mouse ANGPTL4), oligonucleotides may be designed to repress or silence the expression of ANGPTL4, more particularly cANGPTL4. Such oligonuclotides may have any length, but may preferably be short, e.g. less than 100 nucleotides, e.g. 10-40 nucleotides, or 20-50 nucleotides, and may comprise a nucleotide sequence having complete- or near-complementarity (e.g. 80%, 85%, 90%, 91%, 92%, 93%, 94%, 95%, 96%, 97%, 38%, 99% or 100% complementarity) to a sequence of nucleotides of corresponding length in the target oligonucleotide, e.g. the ANGPTL4 mRNA. Repression of ANGPTL4, expression will preferably result in a decrease in the quantity of ANGPTL4, and thus cANGPTL4, expressed by a cell. For example, in a given cell the repression of ANGPTL4 by administration of a suitable oligonucleotide molecule will result in a decrease in the quantity of ANGPTL4 expressed by that ceil relative to an untreated cell. Repression may be partial. Preferred degrees of repression are at least 50%, more preferably one of at least 60%, 70%, 80%, 85% or 90%. A level of repression between 90% and 100% is considered a ‘silencing’ of expression or function. A role for the RNAi machinery and small RNAs in targeting of heterochromatin complexes and epigenetic gene silencing at specific chromosomal loc: has been demonstrated. Double-stranded RNA (dsRNA)-dependent post transcriptional silencing, also known as RNA interference (RNA), is a phenomenon in which dsRNA complexes can target specific genes of homology for silencing in a short period of time. It acts as a signal to promote degradation of mRNA with sequence identity. A 20-nt siRNA is generally long enough to induce gene-specific silencing, but short enough to evade host response. The decrease in expression of targeted gene products can be extensive with 90% silencing induced by a few molecules of siRNA.

In various embodiments, the oligonucleotide molecule is a RNA sequence selected from a “short or small interfering RNA” (siRNA) or a “microRNA” (miRNAs) depending on their origin, Both types of sequence may be used to down-regulate gene expression by binding to complementary RNAs and either triggering mRNA elimination (RNAi) or arresting mRNA translation into protein. siRNA are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences. The oligonucleotide molecule may be formulated as a pharmaceutical composition or medicament.

For example, International patent publication WO 2011046515 A1 discloses siRNAs directed against the ANGPTL4 protein capable of neutralizing or knocking down the protein to halt or reduce cell proliferation and methods of making the siRNA. International patent publication WO 2011046515 A1, which is incorporated by its entirety herein, discloses four sets of siRNAs against human ANGPTL4, as defined in the below Table 4, for ease of reference.

TABLE 4
Examples of siRNAs against ANGPTL4

| SEQ | | |
|---|---|---|
| ID NO | Description | Sequence |
| 17 | siRNA against human | aaagcugcaagaugac |
| | ANGPTL4 sense strand-1 | cucagauggaggcug* |
| 18 | siRNA against human | uaaacagccuccaucu |
| | ANGPTL4 antisense | gaggucaucuugcag* |
| | strand-1 | |
| 19 | siRNA against human | ucgaggcagcaccugc |
| | ANGPTL4 sense strand-2 | gaauucagcaucugca |
| | | uucaagagaugcagau |
| | | gcugaauucgcaggug |
| | | cugcuuuuuuacgcgu |
| | | a* |
| 20 | siRNA against human | agcuuacgcguaaaaa |
| | ANGPTL4 antisense | gcagcaccugcgaauu |
| | strand-2 | cagcaucugcaucucu |
| | | ugaaugcagaugcuga |
| | | auucgcaggugcugcc* |

*“u” in the above RNA sequences is replaced with “t” in the sequence listing, but both refer to uracil.

Accordingly, the oligonucleotide molecules down-regulate the expression of ANGPTL4, and as a consequence cANGPTL4. siRNA ligands are typically double-stranded and, in order to optimise the effectiveness of RNA-mediated down-regulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response, miRNA ligands are typically single-stranded and have regions that are partially complementary enabling the ligands to form a hairpin, miRNAs are RNA genes which are transcribed from DNA, but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double stranded RNA segment. Typically, the RNA ligands intended to mimic the effects of siRNA or miRNA have between 10 and 40 ribonucleotides (or synthetic analogues thereof), more preferably between 17 and 30 ribonucleotides, more preferably between 19 and 25 ribonucleotides and most preferably between 21 and 23 ribonucleotides. In some embodiments of the invention employing double-stranded siRNA, the molecule may have symmetric 3′ overhangs, e.g. of one or two (ribo)nucleotides, typically a UU of dTdT 3′ overhang. Based on the common general knowledge in the technical field, the skilled person can readily design suitable siRNA and miRNA sequences, for example using resources such the Ambion siRNA finder. siRNA and miRNA sequences can be synthetically produced and added exogenously to cause gene downregulation or produced using expression systems (e.g. vectors). In various embodiments, the siRNA is synthesized synthetically.

As will be appreciated, the ANGPTL4 antagonist, which is an oligonucleotide molecule, can be introduced to a subject by gene therapy. Gene therapy refers to therapy performed by the administration of an oligonucleotide molecule to a subject. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo (e.g., ANGPTL4-siRNA). In particular, short antisense oligonucleotides can be imported into cells where they act as inhibitors. There are a variety of techniques available for introducing nucleic acids into viable cells. The techniques vary depending upon whether the nucleic acid is transferred into cultured cells in vitro, or in vivo in the cells of the intended subject. Techniques suitable for the transfer of nucleic acid into cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11, 205-210 (1993)). For example, in vivo nucleic acid transfer techniques include transfection with viral vectors (such as adenovirus, Herpes simplex I virus, lentivirus, retrovirus, or adeno-associated virus) and lipid-based systems.

Accordingly, in various embodiments, the ANGPTL4 antagonist is directed against the nucleotide sequence that encodes ANGPTL4, including the C-terminal fibrinogen-like domain of angiopoietin like 4 (cANGPTL4).

In various embodiments, the ANGPTL4 antagonist is a siRNA molecule. In various embodiments, the siRNA molecule is an ANGPTL4-siRNA molecule, where the siRNA molecule targets a mRNA sequence encoding ANGPTL4. As the mRNA sequence encoding the ANGPTL4 is known, the person skilled in the art would have no difficulty identifying and screening siRNA molecules which specifically target and interfere with the expression of ANGPTL4 with sufficient specificity.

In various embodiments, the ANGPTL4 antagonist is a ANGPTL4-siRNA molecule that targets a mRNA sequence encoding ANGPTL4 and its C-terminal fibrinogen-like domain.

In various embodiments, the ANGPTL4 antagonist is a ANGPTL4-siRNA molecule comprising or consisting of the nucleotide sequence as set forth in SEQ ID NO: 17, or SEQ ID NO. 18, or SEQ ID NO. 19, or SEQ ID NO. 20.

The terms “treating, ameliorating, delaying or preventing”, as used herein refers to achieving one or more of the following in the subject: (a) reducing the severity of a given condition; (b) limiting or preventing the development of a condition; (c) removing a given condition; (d) limiting or preventing the recurrence of a given condition; (e) alleviation of the condition and/or its symptoms; and (f) delay the onset of a condition. Any one or more of these effects may be achieved in a subject who previously had or currently has or is suspected to develop liver fibrosis or a liver disease associated with liver fibrosis. In particular, the therapeutic terms “treating, ameliorating or preventing”, may refer to reducing the likelihood of a particular condition or disease state (e.g., non-alcoholic steatohepatitis) from occurring in a subject not presently experiencing or afflicted with the condition or disease state. The terms do not necessarily indicate complete or absolute prevention or treatment. For example, “preventing NASH” refers to reducing the likelihood of NASH occurring in a subject not presently experiencing or diagnosed with NASH. For example, preventing NASH may reduce the likelihood of NASH occurring in a subject currently diagnosed with mild NAFLD but not currently diagnosed with NASH. The terms may also refer to delaying the onset of a particular condition or disease state (e.g. NASH) in a subject not presently experiencing or afflicted with the condition or disease state. In order to “treat, ameliorate, or prevent” a disease or condition, a method need only reduce the likelihood and/or delay the onset of the disease or condition, not completely block any possibility thereof.

Administration of the ANGPTL4 antagonist, may be done in a variety of ways, including, but not limited to orally, subcutaneously, intravenously, intranasally, intraotically, transdermally, intraperitoneally, intramuscularly, intrapulmonary, vaginally, parenterally, rectally, or intraocularly. The ANGPTL4 antagonist may be formulated accordingly depending upon the manner of administration. In various embodiments, the ANGPTL4 antagonist is formulated as, or comprised in, a composition, more particularly a pharmaceutical composition or medicament for administration to a subject. As will be appreciated, the mode of administration will vary depending on the ANGPTL4 antagonist used and its formulation as a medicament.

Protein therapeutics are often delivered by IV infusion or bolus. The ANGPTL4 antagonist or compositions comprising the same may, in various embodiments, also be delivered using such methods. For example, administration may be by intravenous infusion with 0.9% sodium chloride as an infusion vehicle or carrier.

Medicaments or compositions comprising the ANGPTL4 antagonist may be prepared for storage by mixing the ANGPTL4 antagonist with optional pharmaceutically acceptable carriers, excipients or stabilizers (Remington's Pharmaceutical Sciences 16th edition, Osol, A. Ed., 1980), for example in the form of lyophilized formulations or aqueous solutions. Acceptable carriers, excipients, or stabilizers are pharmaceutically acceptable, i.e. nontoxic to recipients, at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or other immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™, PLURONICS™ or polyethylene glycol (PEG).

In various embodiments, the ANGPTL4 antagonist or composition/medicament comprising the same, may be administered concomitantly with one or more other therapeutic regimens or agents. The additional therapeutic regimes or agents may be used to improve the efficacy or safety of the ANGPTL4 antagonist. Also, the additional therapeutic regimes or agents may be used to treat the same disease or a comorbidity rather than to alter the action of the ANGPTL4 antagonist. The ANGPTL4 antagonist of the present invention may be administered in combination with one or more other prophylactic or therapeutic agents, including but not limited to anti-obesity drugs and anti-diabetes drugs known in the art. The terms “in combination with” and “co-administration” are not limited to the administration of the ANGPTL4 antagonist or therapeutic agents at exactly the same time. Instead, it is meant that the ANGPTL4 antagonist and the other agent or agents are administered in a sequence and within a time interval such that they may act together to provide a benefit that is increased versus treatment with only either the ANGPTL4 antagonist or the other agent or agents. The skilled medical practitioner can determine empirically, or by considering the pharmacokinetics and modes of action of the agents, the appropriate dose or doses of each therapeutic agent, as well as the appropriate timings and methods of administration.

In various embodiments, the ANGPTL4 antagonist is comprised in a pharmaceutical composition or medicament and may be in a water-soluble form, such as comprising certain ingredients in the form of pharmaceutically acceptable salts, such as acid and/or base addition salts. The composition or medicament to be used for in vivo administration are typically sterile. This is readily accomplished by known methods.

In various embodiments, where the ANGPTL4 antagonist is an antibody, the antibody may be formulated as immunoliposomes or in microcapsules. Techniques for preparing such formulations are disclosed in Remington's Pharmaceutical Sciences 16th edition, Osol, A. Ed., 1980.

Accordingly, there is also provided the use of an angiopoietin-like 4 protein (ANGPTL4) antagonist in the manufacture of a medicament for treating, ameliorating, delaying or preventing liver fibrosis or a disease associated with liver fibrosis in a subject.

Further, there is also provided the use of an angiopoietin-like 4 protein (ANGPTL4) antagonist in the manufacture of a medicament for treating, preventing, delaying or ameliorating liver inflammation in a subject suffering from liver fibrosis or a disease or condition associated with liver fibrosis.

The term “effective amount” as used herein refers to an amount of the ANGPTL4 antagonist that, when administered to the subject to treat the condition or disease, will have the intended therapeutic effect, e.g., “treating, ameliorating or preventing”. As will be appreciated, the “effective amount” will vary depending on the ANGPTL4 antagonist used, the severity of the condition, disease and/or its symptoms, as well as the age, body weight of the subject to be treated, and the like. The full therapeutic effect does not necessarily occur by administration of one dose (or dosage), and may occur only after administration of a series of doses. Thus, an effective amount may be administered in one or more administrations. Dosages and administration of an ANGPTL4 antagonist may be determined by one of ordinary skill in the art of clinical pharmacology or pharmacokinetics. An effective amount of the ANGPTL4 antagonist to be employed therapeutically, for example an antibody as described herein, will depend, for example, upon the therapeutic objectives, the route of administration, and the condition of the subject. Accordingly, it will be necessary for the therapist to titre the dosage and modify the route of administration as required to obtain the optimal therapeutic effect. The dosing amounts and frequencies of administration are, in one embodiment, selected to be therapeutically or prophylactically effective. As is known in the art, adjustments for protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

The term “subject”, as used herein in the context of therapeutic methods, refers to a warm-blooded animal, preferably a mammal, more preferably a human. The terms “subject”, “individual” and “patient” are used interchangeably herein and refer to both human and non-human animals. The term “non-human animals” includes all vertebrates, e.g., mammals and non-mammals, such as nonhuman primates, sheep, dogs, cats, horses, cows, chickens, amphibians, reptiles, and the like. In various embodiments, the subject is a non-human. In various embodiments, the subject is a human. Said subject may be awaiting or receiving medical care for is or will become the subject of a medical procedure or is being monitored for the development of liver fibrosis or liver inflammation or diseases associated with liver fibrosis. Subjects include those already being afflicted by or suffering from liver fibrosis or liver inflammation or diseases associated with liver fibrosis and/or liver inflammation as well as subjects susceptible to liver fibrosis or liver inflammation or diseases associated with liver fibrosis or for whom liver fibrosis or liver inflammation or diseases associated with liver fibrosis should be prevented or delayed. In various embodiments, the subject is a human suffering from, or is at risk of suffering from liver fibrosis or liver inflammation or diseases associated with liver fibrosis, more preferably NAFLD, NASH or related disease phenotypes.

However, in another embodiment, rather than administering the ANGPTL4 antagonist in vivo to a subject such as a patient or individual in need of treatment, it is also contemplated that the ANGPTL4 antagonist may be used for in vitro methods for treating liver samples with fibrosis and/or inflammation and/or obtained from a subject suffering from a disease associated with liver fibrosis.

All embodiments disclosed above in relation to the therapeutic methods and ANGPTL4 antagonist similarly apply to the in vitro methods, described below.

Accordingly, there is provided an in vitro method of reducing fibrosis in a liver sample, which comprises the step of contacting the liver sample with an angiopoietin-like 4 protein (ANGPTL4) antagonist.

In another embodiment, there is provided an in vitro method of reducing liver inflammation in a liver sample, which comprises the step of contacting the liver sample with an angiopoietin-like 4 protein (ANGPTL4) antagonist.

In another embodiment, there is provided an in vitro method for regulating an immune response in a liver sample, comprising: contacting the liver sample with an angiopoietin-like 4 protein (ANGPTL4) antagonist, wherein the contacting induces the number, activity and/or effector functions of a population of regulatory T cells, preferably CD8+ T and/or CD4+ T cells, to be reduced in the liver sample.

In various embodiments of the in vitro methods, the ANGPTL4 antagonist is directed against the C-terminal fibrinogen-like domain of angiopoietin like 4 (cANGPTL4).

The terms “sample” or “biological sample” as used interchangeably herein includes any suitable sample isolated from the subject. Suitable samples include, but are not limited to, a sample containing tissues, cells, and/or biological fluids isolated from a subject. Examples of samples include, but are not limited to, tissues, cells, biopsies, blood, lymph, serum, plasma, urine, saliva, mucus and tears. In one embodiment, the sample comprises a serum sample, a blood sample, or a plasma sample. A sample may be obtained directly from a subject or a control (e.g., by blood or tissue sampling) or from a third party (e.g., received from an intermediary, such as a healthcare provider or lab technician). The sample may be obtained from a subject that is suffering from liver fibrosis, liver inflammation or a disease or condition associated with liver fibrosis.

The “contacting” step in the in vitro methods generally refer to any suitable means for delivering, exposing or bringing the sample and the antagonist together in a controlled experimental setting under suitable conditions. As will be appreciated by the skilled person, the “contacting” step can vary depending on the nature of the sample and antagonist involved, whereby the contacting may involve physically mixing the sample with the antagonist in a test tube, petri dish or other container, or it could involve applying the antagonist to the sample through techniques such as pipetting, incubation, or surface immobilization, or it could involve introducing the antagonist into the sample (i.e. cells), such as through transfection. The purpose of the “contacting” step is to allow the antagonist to interact with the sample and exert its inhibitory effect on the target molecule (ANGPTL4). In various embodiments, contacting occurs in a solution in which the sample and ANGPTL4 antagonist are mixed in a common solution and are allowed to freely associate, or the contacting can occur at or otherwise within a cel or in a cell-free environment.

In various embodiments, the liver sample comprises liver cells. In various embodiments, the cells are derived from a liver cell line or obtained from a subject that is suffering from liver fibrosis, liver inflammation or a disease associated with liver fibrosis. In this regard, the cell or cell line may also be maintained or grown in cell culture dishes under suitable conditions, and may be in contact with the ANGPTL4 antagonist (i.e. antibody) in such an environment.

In various embodiments, the contacting step may comprise culturing the cell or cell line in a cell culture in the presence of the ANGPTL4 antagonist, or transfecting a cell or cell line with the ANGPTL4 antagonist.

In various embodiments, the liver sample is a liver tissue. In various embodiments, the liver tissue is obtained from a subject that is suffering from liver fibrosis, liver inflammation or a disease or condition associated with liver fibrosis.

The present invention is further illustrated by the following examples. However, it should be understood, that the invention is not limited to the exemplified embodiments.

### EXAMPLES

### Materials and Methods

Animals, feeding regimens and treatment. Male wild-type C57BL/6J (Invivos, Singapore), LysMCre-Angptl4_{-/-} and NOD scid gamma (NSG, Jackson Laboratory) mice aged between 8-9 weeks were given ad libitum access to LIDPAD (Liver Disease Progression Aggravation Diet, modified from Teklad diet TD. 88137, Envigo, USA) or a control diet (Teklad Custom Diet, modified from AIN-93M, Envigo, USA) (Table 5, below) and water. LysMCre-Angptl4_{-/-} mice have site-specific knockout of Angptl4 expression, primarily in myeloid immune cells. Neutralizing monoclonal antibodies against mouse cAngptl4 (clone 3F4F5) were administered intraperitoneally at 10 mg/kg twice weekly after 4 weeks of NAFLD induction with LIDPAD. Animals were kept in standard housing cages for up to 48 weeks at a controlled temperature and humidity of 30° C. (thermoneutral zone) and 49.9%, respectively, with a 12-hour darklight cycle. Body weight and food consumption were measured weekly. Animals were euthanized using 002 prior to blood and tissue collection. Organs were fixed with 4% PEA or snap frozen with liquid nitrogen. All experiments were carried out following the guidelines of the Institutional Animal Care and Use Committee (IACUC) (SingHealth IACUC: #2014/SHS/1008; NTU-IACUC: A8031, A0032, A8033, A8042, A20055).

TABLE 5
Nutritional Information on Diets
LIDPAD (Liver Disease Progression
Aggravation Diet, Teklad Custom Diet,
1% cholesterol, Envigo)

| Macronutrient Information | % kcal |
|---|---|
| Protein | 11.5 |
| Carbohydrate | 45.2 |
| Fat | 43.3 |

Kcal/g: 4.6

Control (Teklad Custom Diet, Envigo)

| Macronutrient Information | % kcal |
|---|---|
| Protein | 13.7 |
| Carbohydrate | 75.0 |
| Fat | 10.3 |

Kcal/g: 3.6

Patient samples. Ten NAFLD patients and 5 liver cancer patients scheduled for liver surgery in the Fifth Affiliated Hospital of Sun Yat-Sen University were recruited to provide surplus liver tissue excised from the surgery. The patients had no prior or existing liver infection or other active infectious diseases. NAFLD liver tissue samples were obtained from the NAFLD regions of the 10 NAFLD patient livers. Normal liver tissue samples were obtained from the normal tissue surrounding the liver tumours of the 5 liver cancer patients. The obtained samples were then cut in halves and kept either in formaldehyde for histological analysis or in a −80° C. freezer before RNA extraction. The study was approved by the Institutional Review Board of the Fifth Affiliated Hospital of Sun Yat-Sen University, Zhuhai, China (Approval No. L136-1), and written consent was obtained from all the participants.

Liver histopathology. Liver samples were fixed in 4% paraformaldehyde and embedded in paraffin. Histological sections were cut into 5 μm thick sections, dewaxed, and rehydrated before staining with H&E, PSR, and MT via a Leica Autostainer XL (Leica, Germany). Frozen tissue samples for ORO staining were prepared and stained following an isopropanol-based protocol. Images were captured using Axioscan. Z1 (Zeiss, Germany) under brightfield settings at 20× magnification. A blinded histological assessment was performed using the Steatosis, Activity, and Fibrosis (SAF) scoring system described below.

RNA sequencing. Total RNA was extracted from frozen liver samples obtained from all mouse groups and patient samples with TRizol reagent (Life Technologies, U.S.) and E.Z.N.A.® HP Total RNA kit (Omega Biotek, U.S.). RNA samples were sequenced using the Illumina HiSeq platform to generate 50-bp paired-end reads. Raw reads were mapped to the Mus musculus genome assembly from Ensembl, GRCm38 or GRCh38 for human (21), via HISAT2 (22). Uniquely mapped reads were analysed with FeatureCounts (23) to produce gene count matrices, which were subjected to differential expression analysis using DESeq2 (24). Genes meeting the criteria of >±1 log 2-fold-change and adjusted p value<0.05 were considered differentially expressed genes (DEGs). DEGs were stratified by expression and analysed using Gene Ontology and Ingenuity Pathway Analysis (QIAGEN Inc.) to identify the top-ranked enriched pathways. Raw sequences were deposited into GEO with accession numbers GSE159911 and GSE214504.

Single-cell isolation of intrahepatic immune cells. Intrahepatic immune cells were isolated as previously described with some modifications (25). Briefly, mice were anesthetized with 100 mg/kg ketamine and 10 mg/kg xylazine, injected with 3 μg of anti-mouse CD45-FITC antibodies (Miltenyi Biotec, Germany) retro-orbitally and left for 3 min prior to euthanasia by cardiac puncture. The livers were harvested and dissociated into single-cell suspensions using the Liver Dissociation Kit on a GentleMACS Octo Dissociator (Miltenyi Biotec). The cell suspension was filtered through a 70 μm strainer. Lymphocytes in the supernatant were enriched with Percoll density gradient centrifugation (1.07 g/mL) and treated with RBC lysis buffer to deplete red blood cells. The lymphocyte fractions were stained with anti-CD45-APC (panhematopoietic cell marker) and propidium iodide (Live/Dead stain) before sorting on a FACSAria Fusion cell sorter (BD Biosciences, USA). Live intrahepatic immune cells (CD45−FITCCD45−APC+PI−) were collected, washed twice, and counted before single-cell library preparation. Single-cell RNA sequencing and annotation of the scRNAseq immune subpopulation described below.

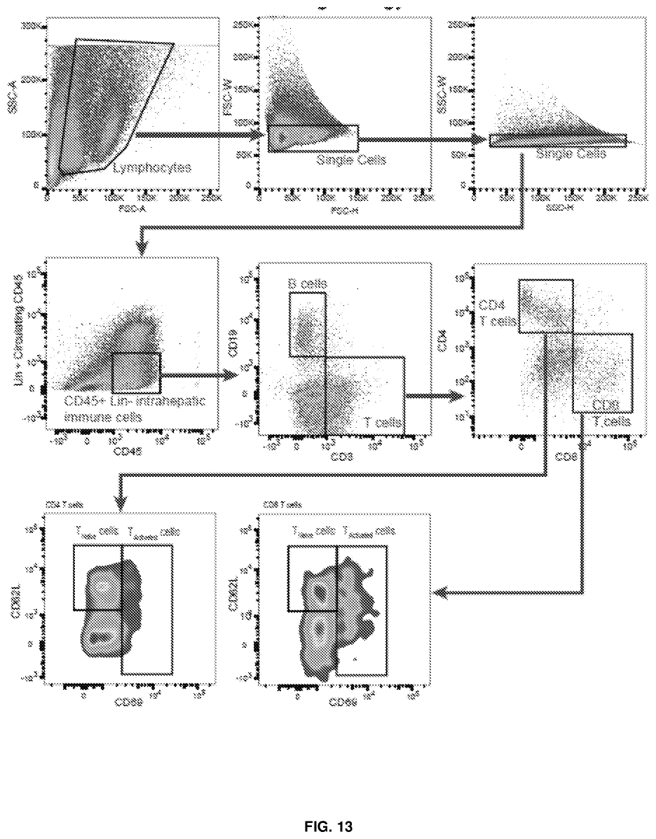

FACS. Intrahepatic immune cells were isolated as mentioned above. Single cells were obtained through a 70 pM strainer, blocked with 3% BSA containing FcR blocker and stained with antibodies (Table 6). The proportions of naïve (CD45₊Lin₋CD3₊CD4₊CD8₋CD62L₊CD69₋) and activated (CD45₊Lin₋CD3₊CD4₊CD8₋CD69₊) CD4₊ T cells, and naïve (CD45₊Lin₋CD3₊CD4₋CD8₊CD62L₊CD69₋) and activated (CD45₊Lin₋CD3₊CD4₋CD8₊CD69₊) CD8₊ T cells were analysed with LSRFortessa X-20 (BD Biosciences, USA) based on the gating strategy in FIG. 13.

TABLE 6
Antibodies used for FACS

| | | | | Catalog |
|---|---|---|---|---|
| Antigen | Fluorophore | Cell type | Company | number |
| CD45 | FITC | Circulating leukocytes | BD | 553080 |
| CD45 | BV785 | Intrahepatic | Biolegend | 103149 |
| | | leukocytes | | |
| CD11b | FITC | Myeloid cells | Miltenyi | 130-113-796 |
| NK1.1 | FITC | Natural killer cells | Biolegend | 156507 |
| Ly6G | FITC | Neutrophils | Miltenyi | 130-102-934 |
| CD19 | APC | B cells | Miltenyi | 130-123-791 |
| CD3 | BV421 | T cells | Biolegend | 100227 |
| CD4 | PE | CD4 T cells | Miltenyi | 130-116-509 |
| CD8b | BV711 | CD8 T cells | Biolegend | 126633 |
| CD62L | APC-Cy7 | Naïve T cells | Biolegend | 104427 |
| CD69 | BV510 | Activated T cells | Biolegend | 104531 |

Kinase inhibitor screen. Splenic CD4₊ T cells were isolated from wild-type and Angptl4_{-/-} murine lymphocytes (methodology as above) using a CD4₊ T-cell magnetic separation column (Miltenyi Biotech). Isolated CD4₊ T cells were cultured in AIM-V culture media (Thermo Fisher Scientific, USA) supplemented with 5% wild-type and Angplt4_{-/-} mouse serum. CD4₊ T cells were treated with a panel of 28 kinase inhibitors (SYN-2103; SYNkinase, Victoria, Australia & TargetMol, Massachusetts, USA) at the stipulated concentrations (Table 7) overnight before treatment with 1 μg/mL LIPS for 2 h. The log 2(fold change) relative expressions of eIF2α, and target genes of eIF2α-mediated downstream genes (i.e., DDIT3, HSPA5 and ATF4) of each kinase inhibitor treatment were compared against LPStreated wild-type conditions via qPCR analysis.

TABLE 7
List of kinase inhibitors used, molecular targets
and concentration used in the drug screen.

| | | Concentration |
|---|---|---|
| Inhibitor | Target | (nM) |
| DMSO | NA | NA |
| Acalabrutinib | BTK | 20 |
| AMG-47a | Lck | 40 |
| AS703026 | MEK1/2 | 44 |
| AZD1208 | Pim1; Pim2; Pim3 | 80 |
| AZD-5438 | CDK1; CDK2; CDK9 | 80 |
| CC-401 HCl | JNK | 200 |
| CID755673 | PKD1; PKD2; PKD3 | 728 |
| CX-4945 | CK2 | 4 |
| Enzastaurin | PKCα; PKCβ; PKCγ; | 24 |
| | PKCε | |
| GSK429286A | ROCK | 760 |
| H 89 diHydrochloride | PKA; S6K1 | 192 |
| IPI-145 | pan-PI3K | 200 |
| KN-62 | CaMK V; CaMKII | 640 |
| KN-93 | CaMK | 1200 |
| Momelotinib | JAK1/2 | 80 |
| Palbociclib Isethionate | CDK4/6 | 64 |
| PD173955-Analog1 | c-Src | 40 |
| PF-562271 | FAK | 6 |
| PF-6260933 | MAP4K4 | 640 |
| Phenformin hydrochloride | AMPK | 40000 |
| R788 (Fostamatinib) | Syk | 164 |
| Disodium | | |
| S-99 | ASK 4 | 4 |
| SB202190 | MAPK (p38); p38αβ | 400 |
| SD169 | MAPK (p38) | 16 |
| SNS-314 | Aurora A/B/C | 124 |
| Tofacitinib | JAK3 | 364 |

Adoptive transfer. Splenic lymphocytes were harvested from Angptl4-knockout mice as described above. Knockdown of eIF2α (or Eif2s1) was performed using 10 nM of Accell mouse Eif2s1 SMARTpool siRNA (Horizon Discovery, UK) based on our published protocol (28). Control cells were transfected with scrambled siRNA. After knockdown, the lymphocytes were labelled with CellTracker™ Green CMFDA Dye (Thermo Fisher Scientific, USA) at 1 μM for 45 mins. A total of 1×10⁷ cells were intravenously injected into mice fed the LIDPAD diet for 12 weeks to create a NASH environment. Intrahepatic immune cells were isolated to examine the proportion of naïve and activated cells in donor (labelled with Cell Tracker Green; FITC_{Hi}) and recipient (unlabelled; FITC_{Lo}) CD4₊ T cells via FACS.

In Vivo Magnetic Resonance Imaging. Prior to in vivo imaging, animals were initially anaesthetized with 2.5-3% isoflurane in combination with medical oxygen and medical air in a dedicated mouse chamber. Isoflurane concentration was reduced following induction to 1-2% during imaging to maintain respiration between 80-90 cycles/minute. Animal's respiration rate and body temperature were monitored through a physiological monitoring system. Respiratory-linked gating with a 50 ms trigger delay was used during imaging. In vivo magnetic resonance imaging (Bruker 9.4 T Biospec) was performed using a 40 mm transmit/receive body coil. High-resolution anatomical T1-weighted images were acquired by a Fast Low Angle Shot (FLASH) sequence with repetition time (TR)-337 ms, echo time (TE) 2.5 ms, flip angle (FA) 30°, averages (AV)-3, image matrix size 256×256 and field of view (FOV)— 40×40 mm. Highresolution anatomical T2 weighted images were acquired using RApid imaging with Refocused Echoes (RARE) sequence with TR-5175 ms, TE-30 ms, FA-90°, AV-2, image matrix size 256×256 and FOV— 40×40 mm. Hepatic proton density fat fraction imaging was performed on LIDPAD (L) and control (C) mice at week 4 (n: L=3, C=3), week 8 (n: L=5, C=4) and week 16 (n, L=4, C=6) using multi-echo Dixon (m-Dixon) pulse sequence with parameters: TR-12 ms, TEs-1.85, 2.08, 2.32, 2.56, 2.80, 3.04, 3.27, 3.51 ms, FA-5°, slices-30, slice thickness-1 mm, images matrix size—256×256 and FOV— 40×40 mm. The m-Dixon imaging data was processed and liver PDFF images were generated using the Fat-Water Toolbox. Identical regions of interest (ROI) were drawn within the liver of all animals by matching anatomical positions. Liver PDFF values were expressed as average percentages and statistical analysis (Mann-Whitney tests) was performed using SPSS software V23.

Histological scoring of liver sections. The steatosis score (S) ranged from 0 to 3 (S0: <5%; S1: 5%-33%, mild; S2: 34%-66%, moderate; S3; >67%, marked). Activity grade (A, from 0-4) is the combination of hepatocyte ballooning (0-2) and lobular inflammation (0-2). Ballooning was scored as 0 (normal polygonal hepatocytes), 1 (round, not enlarged hepatocytes, reticulated cytoplasm), and 2 (2× enlarged hepatocytes with clear cytoplasm and clumping of intermediate fibers). Lobular inflammation was scored as 0 (none), 1 (≤2 foci per 20× field), and 2 (>2 foci/per 20× field). Fibrosis scoring was as follows: stage 0 (F0): none; stage 1 (F1): 1a or 1b delicate or dense zone 3 perisinusoidal fibrosis, respectively, 1c periportal fibrosis only; stage 2 (F2): zone 3 perisinusoidal fibrosis and periportal fibrosis; stage 3 (F3): bridging fibrosis; and stage 4 (F4): cirrhosis. Portal inflammation was noted as absent or present.

Histological preparation of kidney samples. Kidneys were fixed in buffered formaldehyde solution, processed, and embedded in paraffin. Four-micrometer-thick tissue sections were stained with H&E, Periodic Acid-Schiff (PAS) Stain Kit (ab150680), and MT stain, according to the manufacturer's protocol. All slide images were captured using a Carl Zeiss Axio Slide Scanner Z1.

Pancreas harvesting, H&E staining, and islet size determination. Harvested pancreatic specimens were fixed in 4% paraformaldehyde (PFA) for 16 h followed by cryoprotection in 30% sucrose for an additional 24 h. The specimens were embedded and sectioned in Optimal Cutting Temperature compound (OCT, Sakura, Japan) and stained with H&E. The stained slides were imaged using a Carl Zeiss Axio Slide Scanner. Z1 at 20× magnification. Zoomed-in-tif images of the islets were documented together with a digital ruler and annotations. Islets were marked and analysed for size using ImageJ software. The scale was adjusted for each image, and the ROI for each islet, manually marked, was used to calculate the area. Islet areas were then collated and evaluated on GraphPad Prism. Data are presented as the means±SEMs (≥5 islets/animal).

Insulin tolerance test. Mice were injected intraperitoneally (i.p.) with 1 U/kg body weight insulin (Lilly, USA) after 4 h of fasting. Blood glucose concentrations were measured up to 120 min after injection from the tail vein using a glucometer (Accucheck Performa, Roche, USA). All measurements were taken from 10 am-12 noon the next day.

Intraperitoneal Glucose Tolerance Test. Mice were injected intraperitoneally with 2.0 g/kg body weight glucose after 16 h of overnight fasting. Blood glucose concentrations were measured up to 120 min after injection from the tail vein using a glucometer (Accucheck Performa, Roche, USA). All measurements were taken from 10 am-12 noon the next day.

Serum cytokine array. Blood samples collected from the mice via cardiac were left to clot for 30 minutes at room temperature and then transferred to ice. The clotted samples were spun in the centrifuge at 12,000×g for 1 min to separate the serum. The supernatant was carefully retrieved and stored. A mouse cytokine 32-plex discovery assay was then performed by Eve Technologies (Calgary, Canada).

Calorimetric chamber. Mice were prehabituated in the testing room at 30° C. for 5 days, including for 3 days in habituation cages. The climate chamber was maintained at a constant temperature of 30° C. and humidity of 50%. The light was ON at 7 am with 80 lux in the chamber and OFF at 7 μm. Animals had ad libitum access to food (LIDPAD or control accordingly) and filtered water. Oxygen and carbon dioxide sensors were calibrated with calibration gas mixtures (calibration report: good air equivalent: CO₂: 0.050±0.0005%, O₂: 20.900±0.1% in N2; CO₂ span: CO₂: 1.000±0.001%, O₂: 20.000±0.1% in N2). The sample air flow was adjusted to 0.37 L/min. High-precision weighing stations combined with leak- and spill-proof containers recorded the body weight, food intake, and water intake. Spontaneous activity was recorded with two levels of infrared light beam frames surrounding each cage. Recording began from the chamber's first entrance, but measures were considered only from the 2nd day in the chamber. Running wheels were added to the cage from the 5th day. Habituation with running wheels lasted 2.5 days before the data were considered. Measurements were taken every 15 min and are presented here hourly or as the means per hour.

Lipid analyses using liquid chromatography-mass spectrometry. Lipids were extracted from mouse liver tissue using liquid-liquid extraction as described (26) with modifications. Briefly, 10-20 mg of tissue was mechanically homogenized in a methanol:MTBE mixture using bead beating (Precellys). The lysates were further subjected to sonication in an ultrasonic bath with ice for 10 min. Phase separation was induced by adding water, followed by a 10 min incubation at room temperature. The sample was then centrifuged at 1,000×g for 10 min. The upper organic phase was collected, and the aqueous phase was re-extracted. The pooled organic phase was dried under vacuum using a Speedvac, and the lipid extracts were stored at −80° C. prior to further analyses. Samples were spiked with internal standards containing d7-cholesterol (Avanti Lipids), and sterols were analysed using atmospheric pressure chemical ionization (APCI) triple quadrupole mass spectrometry (SCIEX Qtrap 6500+) with upfront liquid chromatography (Agilent Technologies). The separation of sterols was achieved using a Poroshell 120 SB-C18 column (3.0×50 mm, 2.7 μM, Agilent Technologies), which is a modification of a previous method (27). For mass spectrometry analyses, the instrument was operated in multiple reaction mode (MRM). For quantitation, the area under the curve was obtained for each sterol measured and normalized to the internal standard and tissue weight. External calibration was performed for cholesterol and squalene to adjust the response factor for squalene relative to cholesterol.

Immunofluorescence staining. Liver sections were dewaxed, rehydrated and subject to heat antigen retrieval in sodium citrate buffer (10 mM, 0.05% Tween-20, pH 6) using Aptum Biologics 2100 Antigen Retriever (Aptum Biologics, UK). The sections were blocked with 5% (v/v) fetal bovine serum for an hour and labelled with anti-mouse CD45 or anti-mouse α-smooth muscle actin (α-SMA) antibodies (Cell Signaling Technology, USA) at 4° C. overnight. The sections were then treated with Alexa Fluor 680-conjugated secondary antibodies and counterstained with Hoechst 33342. Microscopic images of the sections were captured with Zeiss AxioScan.Z1.

Single-cell RNA sequencing (scRNA-seq). Single-cell droplets and cDNA libraries were prepared using a Chromium Single Cell 3′ V3 kit (10× Genomics, USA) according to the manufacturer's instructions. The cDNA libraries were sequenced on a NovaSeq6000 (Illumina, USA). Raw sequencing reads were aligned to the mm10 mouse reference genome using Cell Ranger (v3.1.0) to generate single-cell count matrices that were normalized, integrated, and annotated using Seurat (v3.0) (28). The obtained count matrices were merged and normalized using SCTransform. Subsequently, cells with fewer than 250 genes, more than 20% mitochondrial genes, and genes with less than 500 UMI counts were filtered out. Average housekeeping gene counts were used as a quality control measure. Datasets were not corrected for cell cycle differences, as visual inspection of the first 2 principal components of cell cycle genes did not show cell cycle bias. Overall, a total of 32551 cells were obtained across all 8 conditions. Subsequently, the dataset is integrated using the Seurat v4 pipeline. The kBET metric revealed the effective removal of the batch effect. Principal component analysis (PCA) was performed on highly variable genes, and the first 35 PCA components were used to construct UMAP. Unsupervised cell clustering was performed with the Louvain algorithm. Cluster marker detection was performed by differentially expressed gene (DEG) analysis for each marker against the remaining markers using FindAllMarkers. Functional annotation of differentially expressed gene sets was performed using EnrichR (29). Trajectory and pseudotime DEG analyses were performed using Slingshot (30) and Trade-seq (31). The single-cell sequencing data were deposited in GEO (Accession number: GSE214172).

Annotation of the scRNAseq immune subpopulation. Cell type annotation was performed automatically using the SingleR package (32) against the ImmGen (33) database, as it performed superior in a recent benchmarking evaluation (34). Subsequently, manual curation of cell types was performed with the top differentially expressed genes of each cluster and compared to automatic annotation to ensure fidelity of annotation (35). T lymphocytes are identified with expression of canonical Cd3 marker—where it is segregated into alpha-beta T-cell receptor (TCR) T lymphocytes and gd-T cells (expressing gamma-delta TCR) via Trac or Trdc markers, respectively. Furthermore, conventional CD4₊ and CD8₊ T cells were identified with Cd4, Cd8a, and Cd8b1 markers. Naïve T cells were identified with L-selectin (Sell) within these populations. Cells that ambiguously expressed CD4 and CD8 markers were identified as doublenegative T cells (dnT). T-innate-like and NKT-cell types are enriched with innate-like markers such as Nkg7 and the Klr gene family. Murine intrahepatic NKT cells specifically express the Cxcr6 marker, which indicates the ability to recognize Cd1d+ antigen presenting cells (36).

B lymphocytes were identified with enrichment of canonical Ms4a1, Cd19, and Cd79a markers. B lymphocytes also express H2-Aa, which are major histocompatibility class 2 (MHC-II) molecules (37) that are functionally involved in antigen presentation to CD4+ T cells during activation. Antibody producing plasma cells were identified with the Igha marker. Innate lymphoid cells (ILCs) are differentiated from NK cells with high expression of Xcl1 and Cxcr6 and lower expression of Nkg7(38). Neutrophils were identified with S100a8 and S100a9 markers (36). Others have reported technical dropout issues in scRNA-seq to identify neutrophils with canonical Ly6g mRNA markers (38). Monocytes express Fcgr1, Fcgr3, and Mafb and lack the Itgax activation marker. Kupffer cells are differentiated from monocyte-derived macrophages (mo-macs) with exclusive expression of Clec4f+ and Csf1r+ (39). Finally, classical dendritic cells (cDCs) were identified with Clec10a and H2-Aa, while plasmacytoid DCs (pDCs) were identified with Tcf4 and Irf8 markers (40,41).

### Results

### Example 1: LIDPAD Mice Show Transitory Stages that Mirror Human NAFLD and Extrahepatic Complications

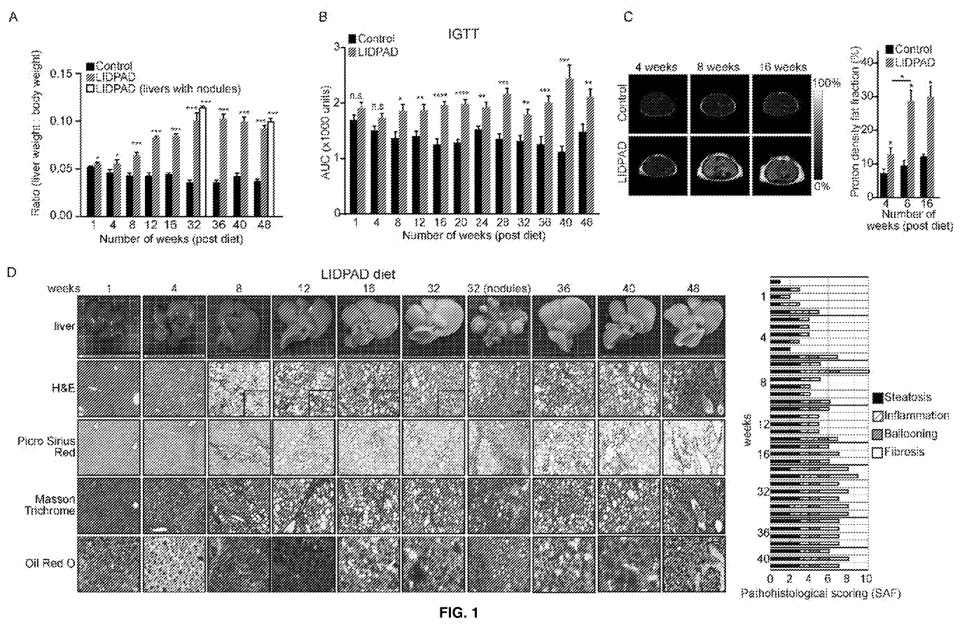

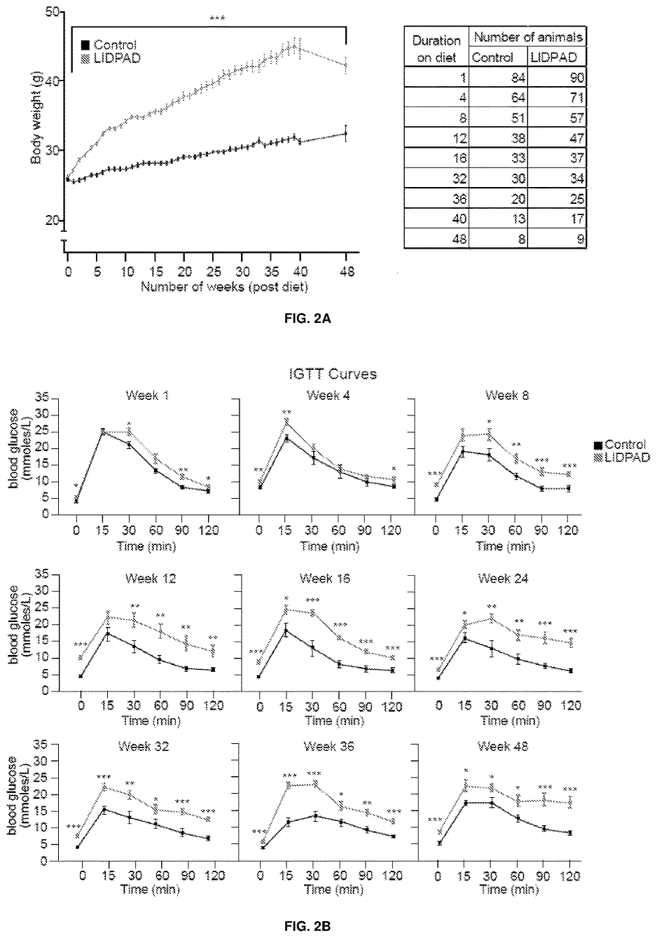

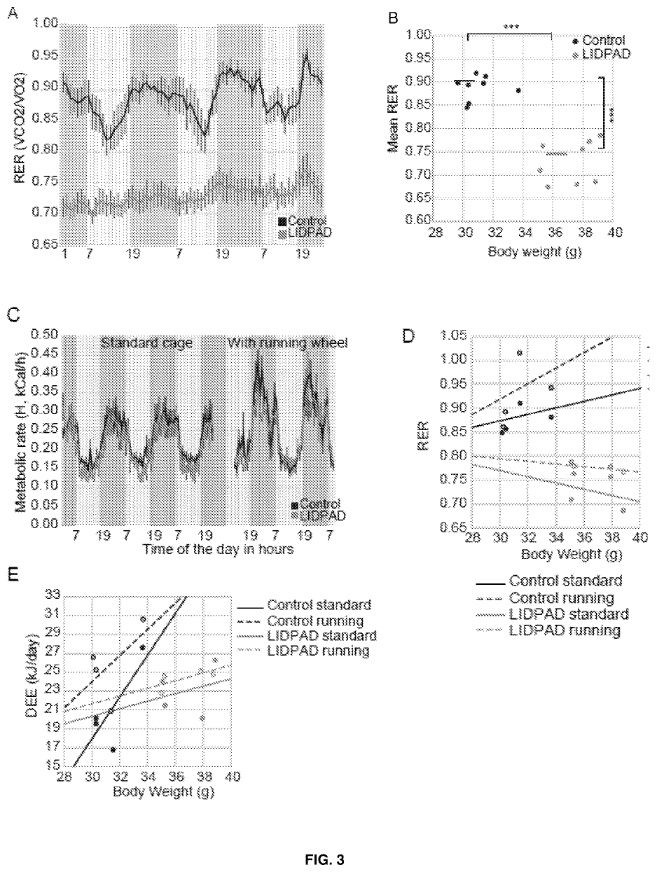

Wild-type C57BL/6J male mice housed in the thermoneutral zone (30° C.) were fed either a modified high fat-cholesterol diet of refined ingredients, herein called LIDPAD mice (Liver Disease Progression Aggravation Diet), or a matched Control diet with refined ingredients to ensure comparability (Table 5). LIDPAD mice showed a gain in body and liver weight as early as 1 week into the diet, which increased progressively compared with the control group (FIG. 1A, 2A). Whole-body calorimetric analysis revealed that LIDPAD mice displayed impaired metabolic flexibility, with less obvious circadian oscillation between carbohydrates and lipid utilization with a stable low respiratory exchange ratio (RER 0.70-0.75), indicating a preference for lipids as fuel compared with control mice (FIG. 3A-B). The daily energy expenditure shift was also lower in LIDPAD mice when running wheels were introduced, indicating that LIDPAD mice exercise less than control mice (FIG. 3C-E).

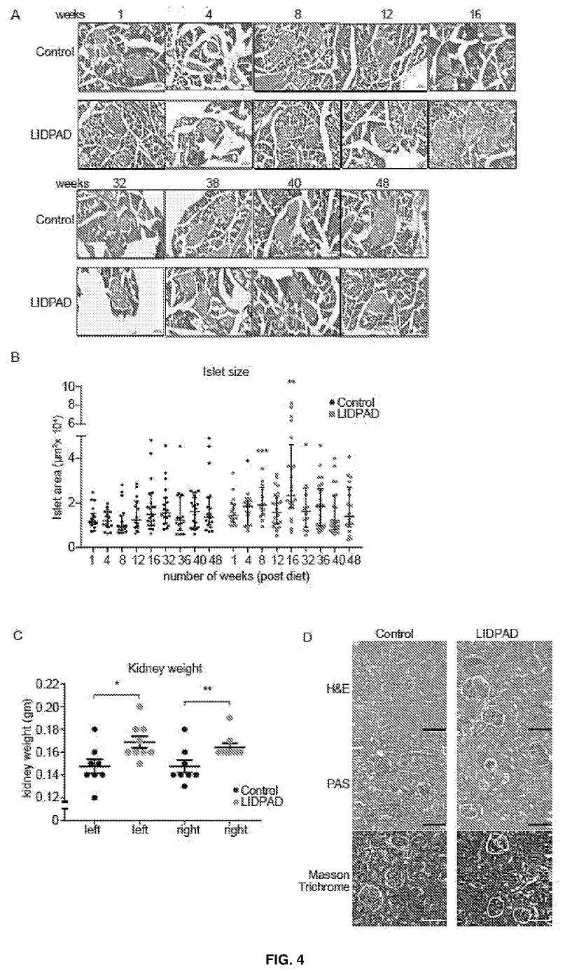

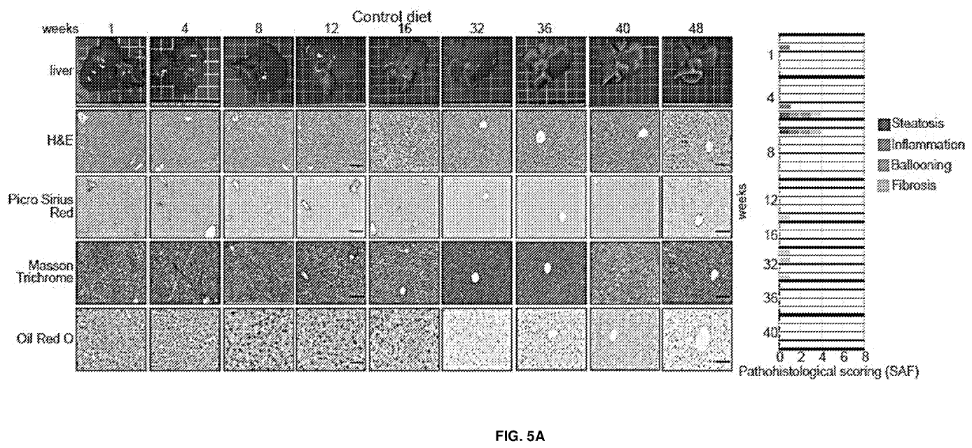

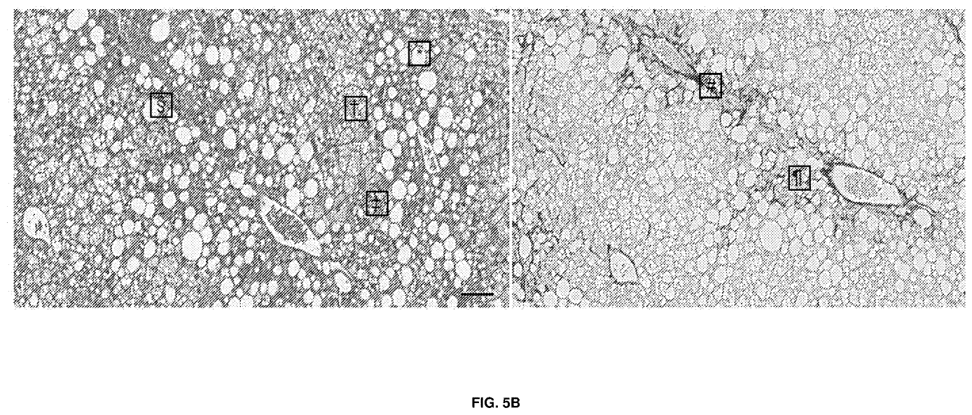

LIDPAD mice developed glucose intolerance within 8 weeks, which corresponded to changes in pancreatic islet size (FIG. 1B, 2B). The islet area in the control group showed no significant difference from week 1 to 48, with an average islet area of 0.5 μm₂×10₄ (FIG. 4A-B). In contrast, in LIDPAD mice, there was a significant increase (1.5-fold) in the islet area between weeks 4 and 16, corresponding to times when glucose intolerance became evident, suggesting compensation via islet cell hyperplasia, consistent with islet compensation during pre-diabetes. From 40 weeks onwards, smaller islets (compared with control mice) started to appear (≤0.5 μm₂×10₄) in LIDPAD mice, although the islet area remained widely distributed, corresponding to perhaps a deterioration of islet mass because of prolonged insulin resistance, mirroring associations between NAFLD and the development of type-2 diabetes. Nephromegaly characterized by an enlarged interstitial space, increased immune cell infiltration, and mesangial expansion was also observed in LIDPAD mice at 48 weeks (FIG. 4C-D). Kidney sections also showed fibrosis, suggesting prior renal injury. Given the impaired ability of these mice to metabolize glucose, kidney enlargement could result from diabetic nephropathy.

LIDPAD and control mice underwent magnetic resonance imaging-based proton density fat fraction (PDFF) of liver at 4-, 8- and 16-weeks post-diet initiation. At all imaging timepoints, LIDPAD mice had a significantly increased liver fat fraction compared with control mice (FIG. 1C). LIDPAD-fed mice displayed a hepatic PDFF ranging from 12.9% at 4 weeks post-diet to 30.13% at 16 weeks post diet. A 2.2-fold increase (12.9 to 28.9%) in the liver fat fraction of the LIDPAD mice was measured between the 4 and 8 weeks imaging timepoints. Hepatic lipid accumulation slowed between 8 and 16 weeks, with the PDFF rising by only 1.3% (FIG. 1C). Control mice showed an expected slight increase in liver fat fraction across the 4-, 8-, and 16-weeks imaging timepoints, with PDFF ranging from 7.4 to 12.5% (FIG. 1C).

Histological liver examination remains the gold standard for diagnosing NASH. Liver sections of LIDPAD and control mice at various weeks of diet were scored by pathologists using the Steatosis, Activity, and Fibrosis (SAF) system. Hepatic lipid accumulation was evident as early as 1 week of LIDPAD feeding but was absent in the livers of control mice (FIG. 1D, 5A), consistent with the MRI imaging data. After 4 weeks, the livers of LIDPAD mice were pale and enlarged with intracellular lipid vacuoles, supported by an SAF score of either grade 2 or 3 in all liver specimens. By 8-12 weeks, mice under LIDPAD developed widespread steatosis, varying levels of intralobular inflammation and infiltration of lymphocytes and macrophages, similar to what is observed in human NASH. In particular, hepatocyte ballooning, the characteristic feature of NASH was seen by 8 weeks. (FIG. 1D, 5B). Fibrosis was prevalent in 50-80% of the mice (FIG. 1D). With prolonged feeding of LIDPAD, an increased severity of hepatic fibrosis and more extensive lipid accumulation were observed, except for weeks 32 and 48 (FIG. 1D, 5A). These instances of reduced steatosis were likely due to decreased lipid accumulation when fibrosis became more prevalent. Liver specimens from weeks 16 through 40 indicated a sustained and consistent increase in all aspects of steatosis, ballooning, inflammation, and fibrosis (FIG. 1D, 5A, B). From and beyond week 32, hepatocellular nodules were observed in ~13% of the mice, comparable to the rate of ~2-12% for human hepatocellular carcinoma (42).

Altogether, LIDPAD mice display a wide array of physiological and physical activity impairments. The LIDPAD mouse model presents the entire spectrum of NAFLD disease progression, with steatosis starting in the early timepoints (weeks 1-4) and NASH appearing after 8 weeks. Fibrosis was noted in 80% of mice starting at 12 weeks. A proportion of LIDPAD mice eventually develop end stage sequelae such as cirrhosis and hepatocellular nodules, mirroring NAFLD patients in disease progression and incidence.

### Example 2: Hepatic Transcriptomic Analyses and Cytokine Profiling Highlight Hepatic Inflammation and Fibrosis

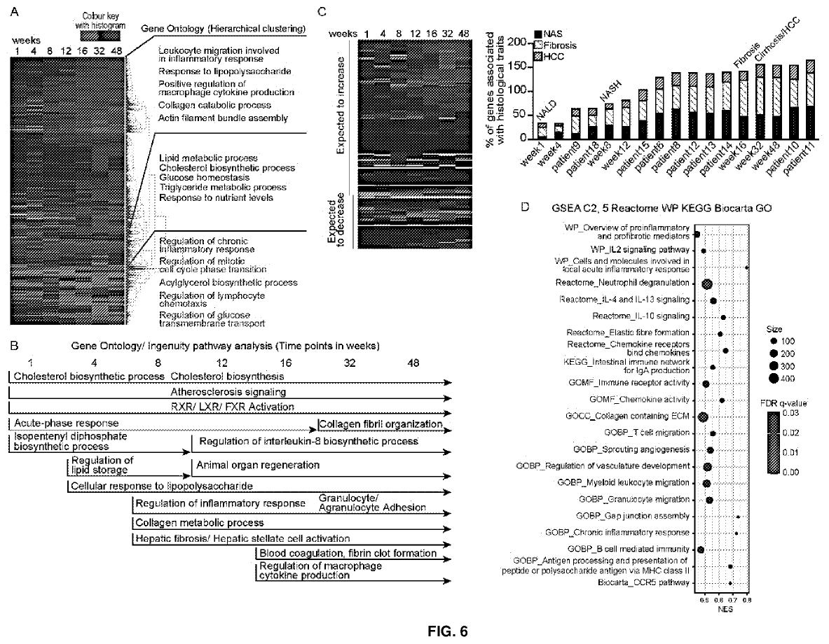

To identify key cellular mechanisms that underpin the observed histological changes in NAFLD, transcriptomic and biochemical analyses was performed. Hierarchical clustering of differentially expressed genes (DEGs) across all LIDPAD liver samples revealed three gene groups according to their expression trends with diverse Gene Ontology profiles (FIG. 6A-B). Among the prominent early events were the dysregulation of cholesterol and lipid homeostasis, as well as the acute-phase response, which indicates hepatic inflammation in response to lipotoxicity. LIDPAD mice also have a hepatic transcriptomic profile of altered cellular response to LPS between weeks 1-4, suggesting a compromised gut barrier. As NASH developed, genes involved in collagen assembly and inflammatory response pathways appeared and intensified, concurring with the histology and pathological scoring for NASH (FIG. 6A-B).

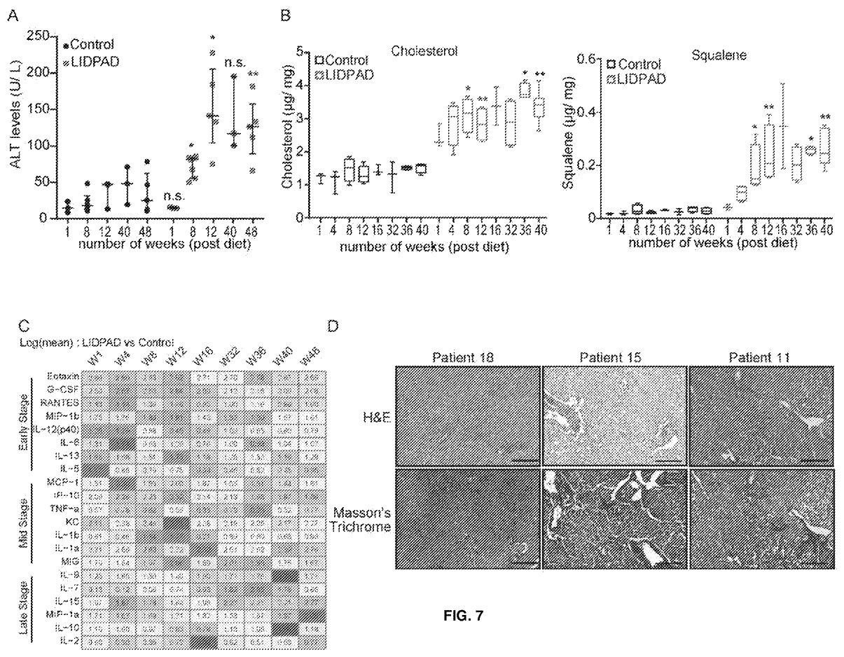

The above findings were confirmed with a series of biochemical measurements. A significant increase in alanine transaminase (ALT) levels, as a biomarker of liver damage, was observed in LIDPAD mice beginning at week 8 of the diet (FIG. 7A). Although there was a mild increasing trend of ALT in control mice during aging, this increase was not statistically significant. Corroborating the transcriptomics analysis, both free cholesterol and squalene levels were elevated in the LIDPAD mice compared to the controls, as determined by LC-MS (FIG. 7B). A basal amount of cholesterol was detected in the livers of control mice, a site for cholesterol synthesis, whereas squalene levels were close to none (FIG. 7B). The increase in free cholesterol can be attributed to cholesterol in the LIDPAD diet. The elevation in the concentration of squalene, a precursor of cholesterol, suggests that the sterol biosynthetic pathway was affected by LIDPAD.

Various studies have identified serum cytokines in the evaluation of NAFLD patients (43). Multiplex immunoanalysis of serum revealed that no single cytokine remained elevated throughout the progression of steatosis to cirrhosis. We observed distinct yet overlapping early-, mid-, and late-stages of cytokine profiles (FIG. 7C). At weeks 1 to 4, we observed an increase in low-grade chronic inflammatory cytokines (IL-6 and IL-13) and inflammatory chemokines (MIP-1α and eotaxin), consistent with the start of steatosis and liver damage. At weeks 8-12, the inflammatory response was sustained by a new group of cytokines (TNF-α and IL-1α) and chemokines (MCP-1 and MIG) that further promoted the infiltration of immune cells. After 12 weeks, the early- and mid-stage cytokines decreased and were replaced by another group of cytokines that are important for the maturation of adaptive immune cells (IL-7, IL-9, IL-10, and IL-2) (FIG. 7C). While cohort studies have identified similar serum biomarkers, they failed to address the temporal changes as disease progresses. Thus, different panels of cytokines/chemokines appear useful for monitoring the progression of NAFLD.

Next, the LIDPAD model results were compared to a human meta-analysis of NAFLD transcriptomic signatures of 218 genes associated with histological worsening (44). The expression of most genes detected in the LIDPAD model exhibited the same-sign consistency compared to human gene expression (FIG. 6C). Guided by histological examination and major shifts in temporal gene expression, it was revealed that the weeks 1, 8, 16, and 32 of LIDPAD feeding corresponded to key transition phases of human NAFLD progression (FIG. 6C). Transcriptomic-guided staging of 10 NASH patients was performed to assess the robustness of the LIDPAD transcriptomic progression signature, which revealed that most of the patient's liver biopsies (6 out of 10) corresponded to weeks 12 to 16 of the LIDPAD mouse model, i.e., NASH with fibrosis (FIG. 6C). Histological evaluation of matched liver specimens showed some degree of concordance based on ordinal SAF staging (FIG. 7D). Gene set enrichment analysis (GSEA) of the human NASH transcriptomes revealed that biological processes such as hepatic inflammation, fibrosis and vascular perturbations predominated in human NASH livers (FIG. 6D).

### Example 3: Angptl4 Deficiency Alters the Intrahepatic Immune Cell Landscape to Delay Liver Fibrosis

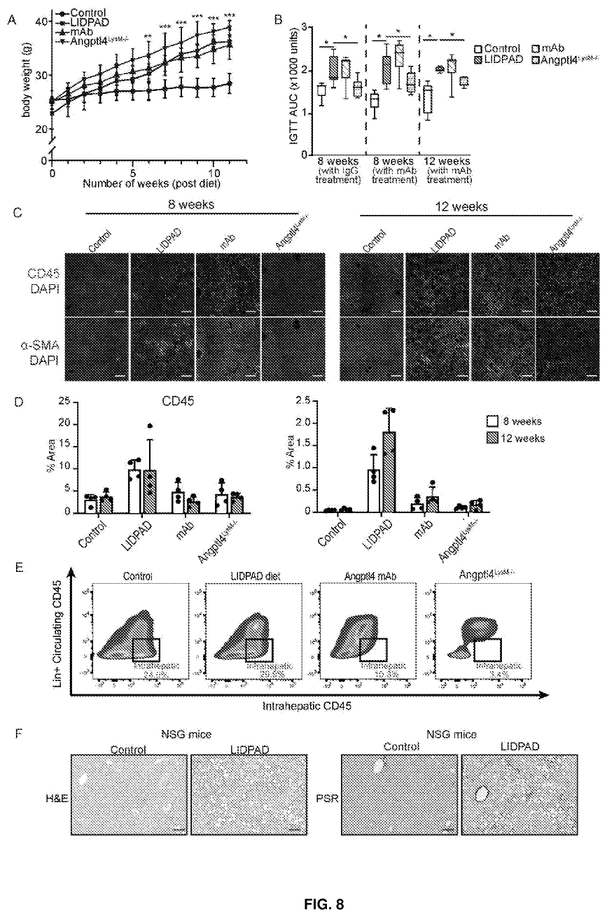

To understand the role of Angptl4 in the immunopathogenesis of fibrosis, the effect of Angptl4 deficiency was examined in two models: myeloid cell-specific Angptl4 mutant mice (Angptl4_{LysM-/-}) and mice treated with a neutralizing antibody (mAb) against cAngptl4 during LIDPAD induced NASH (28, 32). Mice at 4 weeks of LIDPAD feeding were treated with mAb. These mice continued to gain weight similar to control mice. mAb did not alter glucose intolerance induced by LIDPAD feeding (FIG. 8A-B). Similarly, Angptl4_{LysM-/-} mice gained weight but had significantly higher glucose tolerance across all conditions. These observations suggest that immunoneutralization of cAngptl4 does not affect glucose disposal following bolus administration, whereas genetic Angptl4 deletion in myeloid cells improves whole body glucose tolerance.

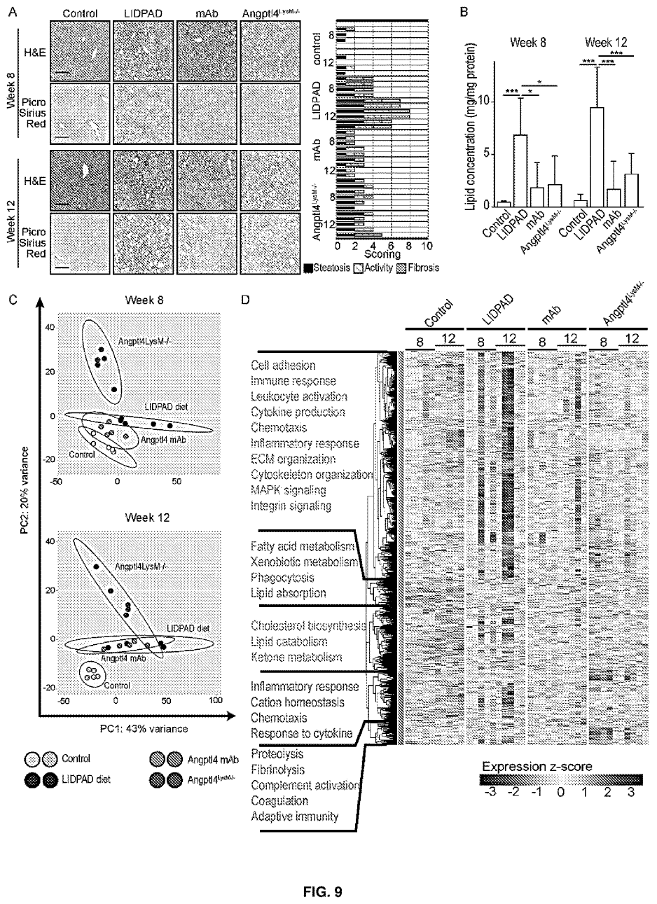

At 8 weeks of LIDPAD, wild-type mice develop steatosis and inflammation in the liver, with increased prevalence and severity of NASH and fibrosis by week 12. Notably, both the mAb treatment and Angptl4_{LysM-/-} mice had reduced hepatic inflammation and fibrosis (FIG. 9A-B, 8C-E). Hepatic transcriptomic profiles confirmed the ameliorative effects of targeting Angptl4 to delay NASH progression. Principal component analysis (PCA) revealed that the liver transcriptomes of mAb-treated LIDPAD mice occupy the space between control and LIDPAD mice, with diminished differences between mAb-treated LIDPAD mice and LIDPAD mice on longer feeding, suggesting a slower disease onset in the former (FIG. 9C). The hepatic transcriptomes of Angptl4_{LysM-/-} mice formed a distinct cluster from other groups that shifted toward LIDPAD mice with prolonged feeding, suggesting a different hepatic impact from the combined effects of gene ablation and high-calorie feeding. DEG analysis identified five functional clusters (FIG. 9D). As expected, the LIDPAD diet markedly activated the hepatic inflammatory response and ECM remodelling activities, which became stronger with prolonged feeding. These proinflammatory and profibrotic events were effectively diminished by cAngptl4 mAb, leading to slower progression. While most of the NASH-related immune response was suppressed in Angptl4_{LysM-/-} mice, different sets of proinflammatory genes that were associated with fibrinolysis, complement activation, and the adaptive immune system were triggered in these mice, highlighting the interrelationship between innate and adaptive immune responses.

This analysis revealed an immunomodulatory role of cAngptl4 and the benefits of targeting cAngptl4 to delay diet-induced NASH.

### Example 4: Single-Cell Transcriptomics Reveals Adaptive Immune Cell Remodeling During Fibrosis

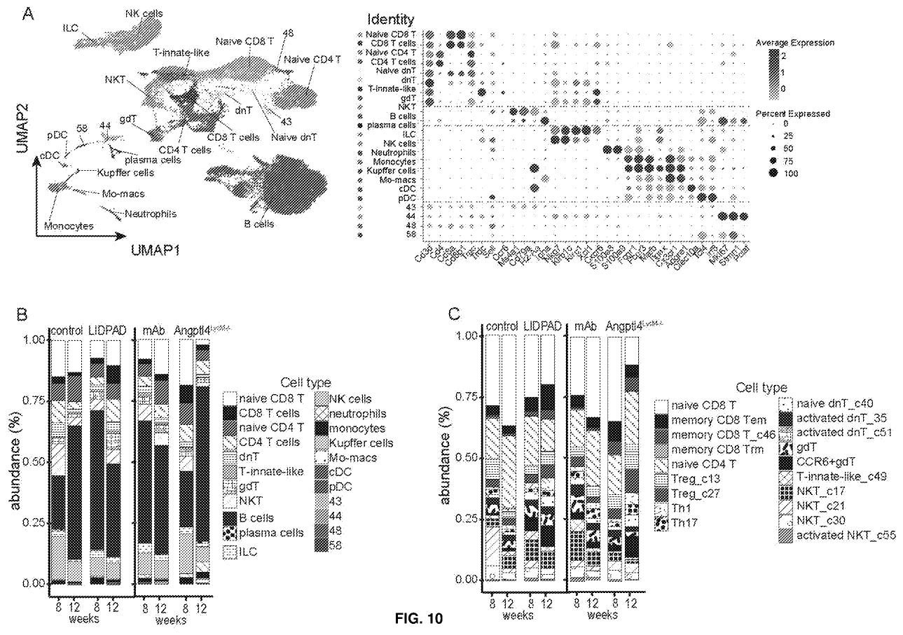

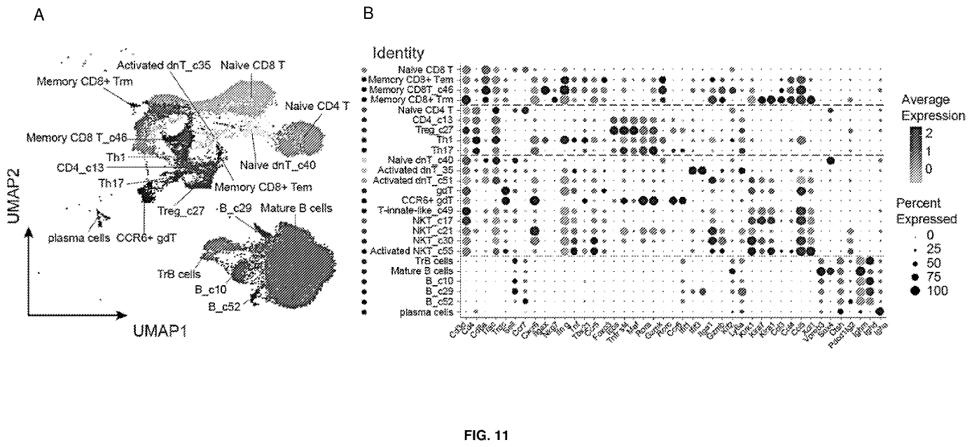

Immuno-deficient NSG mice developed steatosis with only mild fibrosis after chronic (1 year) LIPDAD feeding, confirming the essential prominent role of immune cells in fibrosis (FIG. 8F). To gain insight into the immune cell landscape as NASH livers develop fibrosis, scRNA-seq of intrahepatic immune cells from LIDPAD and control livers was performed at weeks 8 and 12. Nineteen immune cell subpopulations were identified, with B and T lymphocytes constituting most of the intrahepatic immune cell population. B lymphocytes (differentiated and matured plasma cells) and T lymphocytes (8 major cell subclusters) made up ~43.2% and ~41.2% of the total intrahepatic immune cells, respectively. Six subpopulations of myeloid-derived immune cells, namely, neutrophils, monocytes, Kupffer cells, monocyte-derived macrophages (mo-macs), classical dendritic cells (cDCs) and plasmacytoid DCs (pDCs), accounted for close to 10% of the CD45₊ cells in the livers (FIG. 10A-B, Table 8).

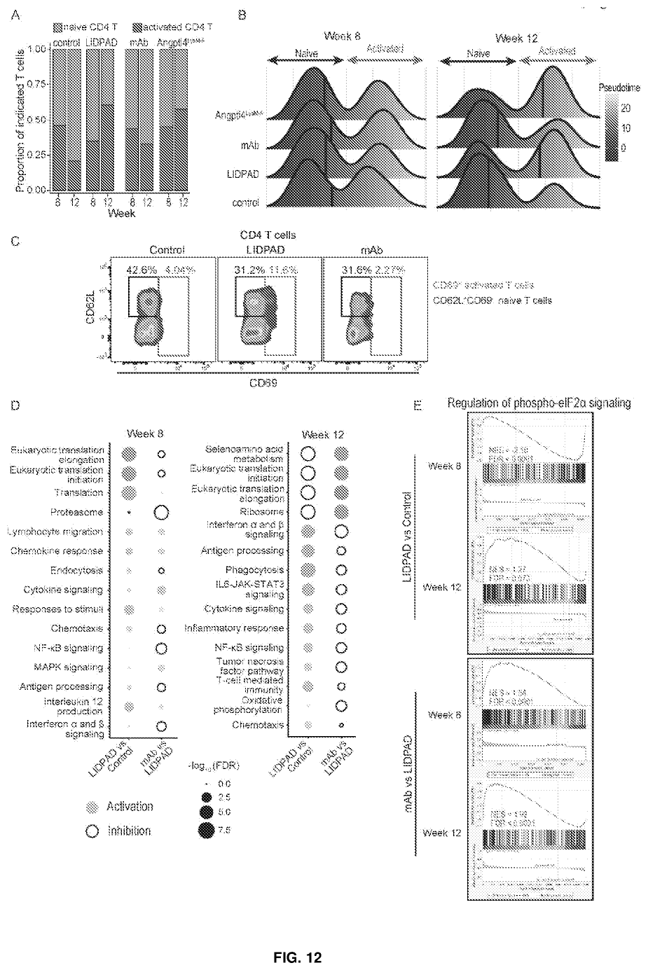

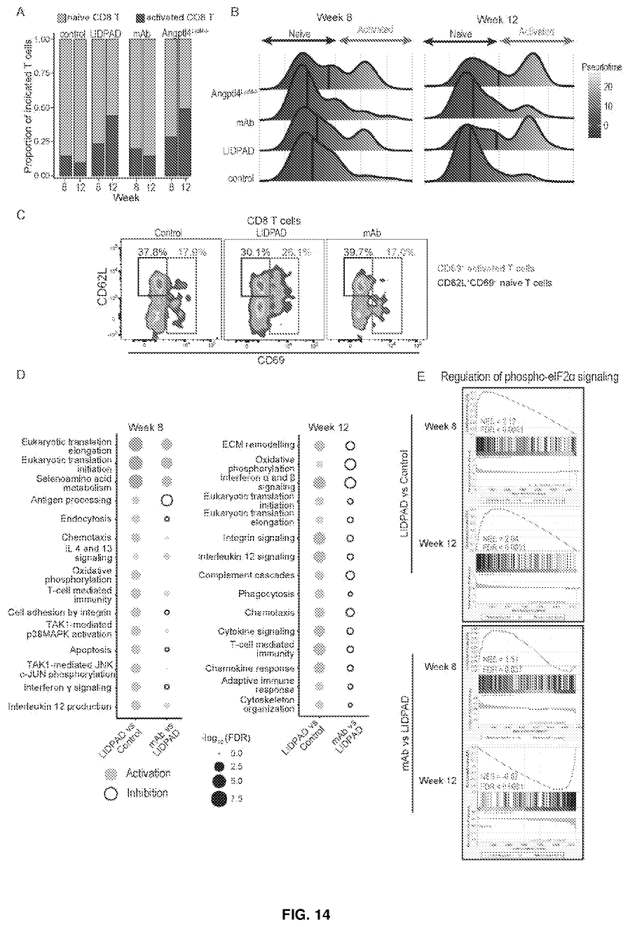

Most myeloid-lineage cells were marginally increased in NASH livers at weeks 8 and 12 compared with the control diet (FIG. 10B, Table 8). On the other hand, B lymphocytes increased at week 8 and decreased at week 12. The proportion of CD8₊ T, CD4₊ T, dnT, and NKT cells showed an opposite trend compared with the control diet, i.e., decreased at week 8 and increased at week 12, suggesting their involvement in fibrosis. High-resolution analysis of the T-cell subpopulations revealed 4 subclusters of CD8₊ T cells, 5 subclusters of CD4₊ T cells, 3 subclusters of dnT cells, 5 subclusters of innate-like T and NKT cells, and 2 clusters of gdT cells (FIG. 10C, 11A, Table 9). CD4₊ and CD8₊T cells demonstrated dynamic shifts in proportions across conditions, accounting for approximately two thirds of the T-lymphocyte population (FIG. C). We interrogated the fate of T-cells to further understand their role. As the disease progressed from 8 to 12 weeks, more naïve CD4₊ T cells were activated, as evidenced by scRNA-sequencing and FACS analysis (FIG. 12A-C, 13). Pseudotime analysis revealed two major peaks along the CD₊ T-cell differentiation trajectory that correspond to the activation state (FIG. 12B). Similar shifts in intrahepatic CD8₊ T cells were also observed (FIG. 14A-C). In summary, fibrosis progression is marked by remodelling of the lymphoid-lineage immune cell landscape, particularly an increase in activated CD4₊ and CD8₊ T cells.

In LIDPAD-fed Angptl4_{LysM-/-} mice, there was a higher neutrophil persistence and macrophage:monocyte ratio than in LIDPAD-fed mice, suggesting a prolonged innate inflammatory response. This also revealed an interrelationship between innate and adaptive immune responses during fibrosis. The directionality of change in the relative abundance of T and B cells was opposite to LIDPAD mice and similar to control mice, i.e., total B cells increased, and T cells decreased from week 8 to 12. (Table 8). However, no change in the activation status of CD4₊ and CD8₊ T cells was observed compared with LIDPAD-fed mice. The mAb treatment of LIDPAD-fed mice reduced the abundance of many immune cells, e.g., neutrophils, mo-macs and monocytes, to levels similar to the control mice. mAb treatment attenuated the relative abundance of T and B cells from weeks 8 to 12 compared with LIDPAD mice (Table 8-9). Importantly, and in contrast to Angptl4_{LysM-/-} and LIDPAD-fed mice, mAb treatment effectively blocked the increase in activated CD4₊ and CD8₊ T cells from weeks 8 to 12 (FIG. 12A-C, 14A-C). Hence, the activation of CD4₊ and CD8₊ T cells is a key event in fibrosis, which was diminished by blocking cAngptl4.

GSEA of CD4₊ and CD8₊ T cells revealed gene sets in two primary processes, namely, eukaryotic translation and immune responses (FIG. 12D, 14D). During fibrosis, a downregulation of eukaryotic translation-associated genes was observed, accompanied by a concomitant upregulation of genes involved in the inflammatory response in CD4₊ T cells (FIG. 12D). In CD8₊ T cells, the eukaryotic translation gene sets became insignificant by week 12, while the genes involved in cell recognition and cytokine mediated responses were upregulated (FIG. 14D). These observations suggest that the regulation of eukaryotic translation activities is pivotal for the proinflammatory phenotypes of the two T-cell lineages.

mAb treatment repressed eukaryotic translation machineries in CD4₊ and CD8₊ T cells, accompanied by substantial inhibition of inflammatory activities in both CD4₊ and CD8₊ T cells at week 12 (FIG. 12D, FIG. 14D-E). The effect of mAb on eIF2α signalling was observed in CD8₊ T cells at week 8 (FIG. 14E), which is likely due to the complex activation mechanism of CD8₊ T cells that require inputs from CD4₊ T helper cells. Using a curated gene set from StringDB and Reactome, a stimulatory effect of the cAngptl4 mAb on the phosphorylated eukaryotic initiation factor-2α (phospho-eIF2α)-mediated signalling was identified, which is often associated to global translation attenuation and could contribute to the suppressed T-cell activation in mAb-treated mice (FIG. 12E).

### Example 5: CANGPTL4 Deficiency Modulates T-Cell Activation by Suppressing Translation Initiation in CD4+ T Cells

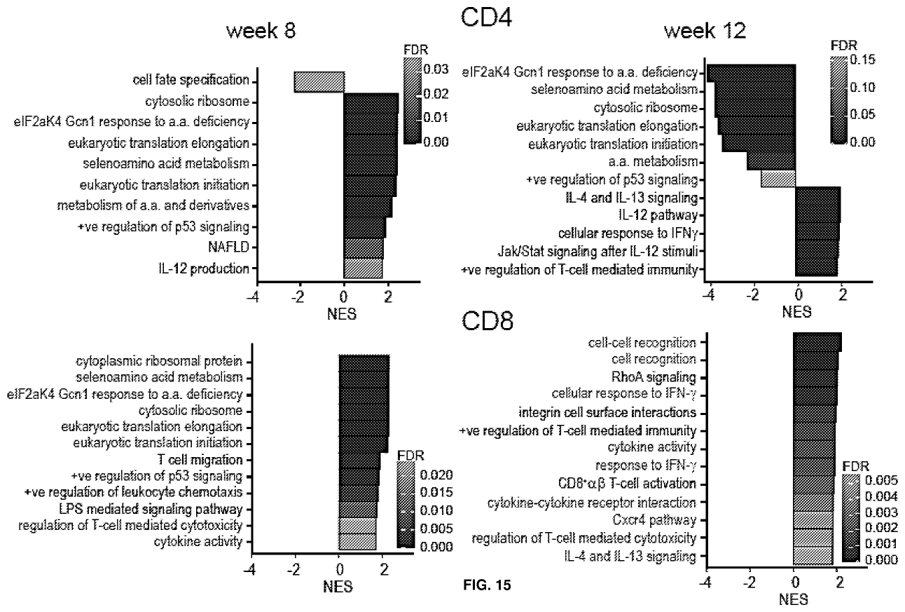

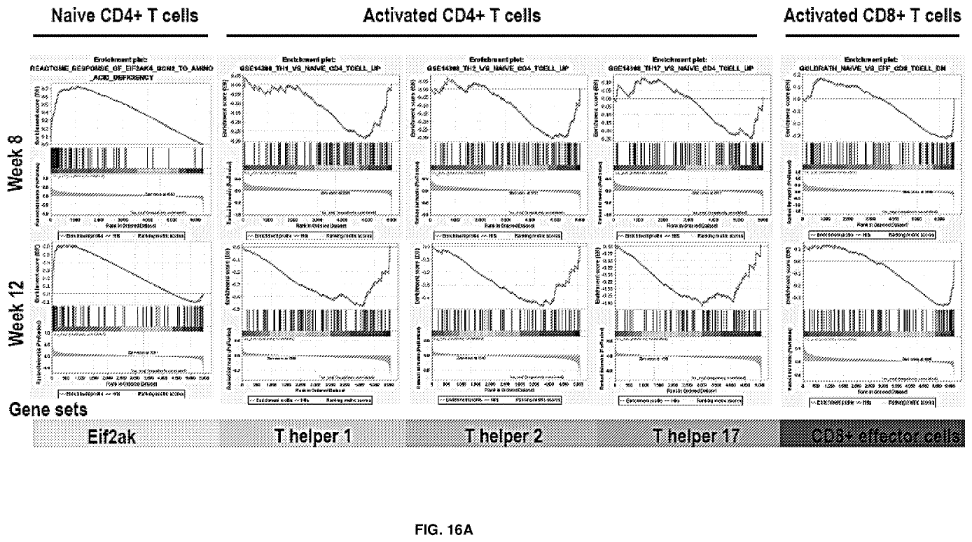

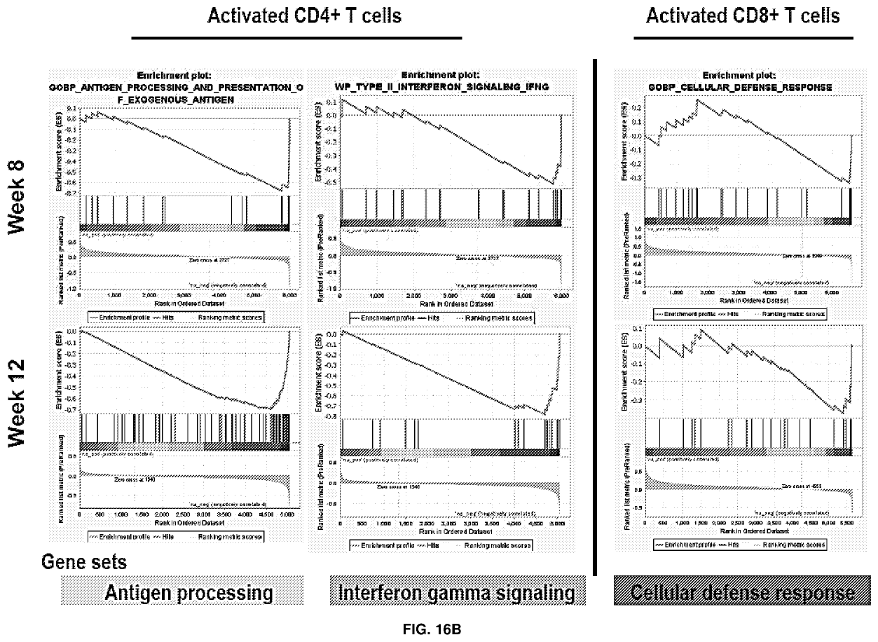

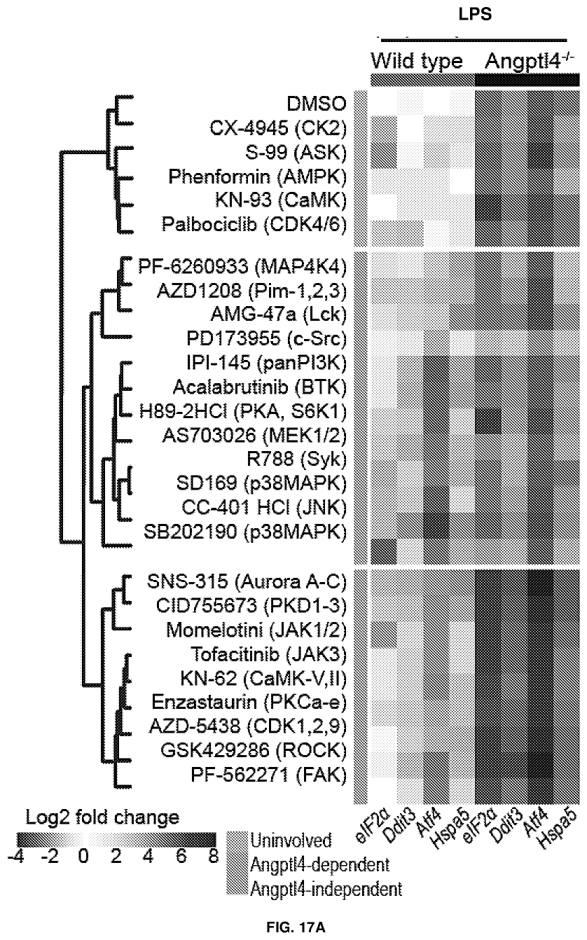

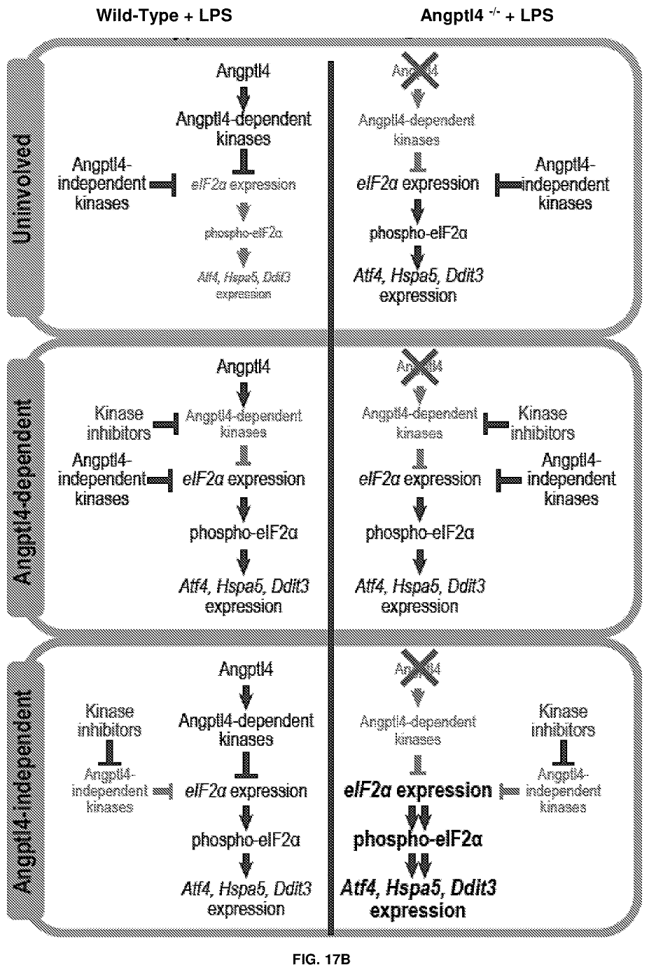

To understand how cANGPTL4 mediates T-cell activation, GSEA of CD4₊ T and CD8₊ T cells was performed which revealed gene sets in primarily two processes, namely in eukaryotic translation and immune responses. With increased disease severity, a downregulation of eukaryotic translation associated genes was observed, accompanied by a concomitant upregulation of genes involved in immune response in CD4₊ T cells. These two gene sets became insignificant by week 12 and were replaced by genes involved in cell recognition and cytokine-mediated responses usually observed in CD8₊ T cells (FIG. 15). These observations suggest distinct roles for CD4₊ and CD8₊ T cells in NASH severity and liver fibrosis.

Upon treatment with cANGPTL4 monoclonal antibodies, the Eif2ak4 signalling response, which suppresses global protein translation, was markedly activated in CD4₊ T cells. In contrast, the proinflammatory signalling pathways demonstrated the reverse trend.

During NASH progression from weeks 8 to 12, many immune- and inflammatory-associated gene sets, including cytokine-cytokine receptor interactions and the IL-4, IL-1, IL-13 and IFNγ signalling pathways, were consistently upregulated in both CD4₊ and CD8₊ T cells. AnticANGPTL4 monoclonal antibodies effectively silenced proinflammatory signalling and antigen processing activity in T cells.

When zooming into T cells of different activation statuses, it was found that Eif2ak4 signalling is specifically activated by cANGPTL4 monoclonal antibodies in naïve CD4₊ T cells, whereas the gene signatures of effector CD4₊ (i.e., Th1 and Th17) and CD8₊ T cells were markedly suppressed in activated T cells, regardless of disease progression (FIG. 16A). The activation of Eif2ak4 signalling in naïve CD4₊ T cells hinders global protein synthesis, preventing subsequent maturation into more specialized lineages. Likewise, the differentiation of effector CD8₊ T cells was hampered since CD4₊ T cells are crucial mediators of CD8₊ T cell activation. Functionally, cANGPTL4 monoclonal antibodies diminished interferon-γ signalling and antigen processing activities in activated CD4₊ T cells, both of which are linked to the immune-modulatory capacity of CD4₊ T cells (FIG. 16B). In activated CD8₊ T cells, although interferon-γ signalling was unaffected by cANGPTL4 deficiency, the defence response was inhibited.

### Example 6: cAngptl4-Mediated eIF2α Signaling Modulates the Activation of CD4+ T Cells

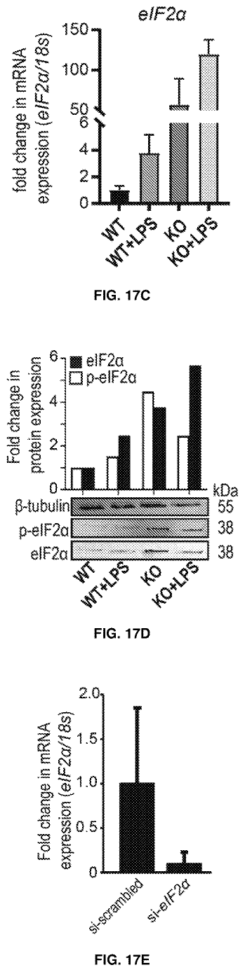

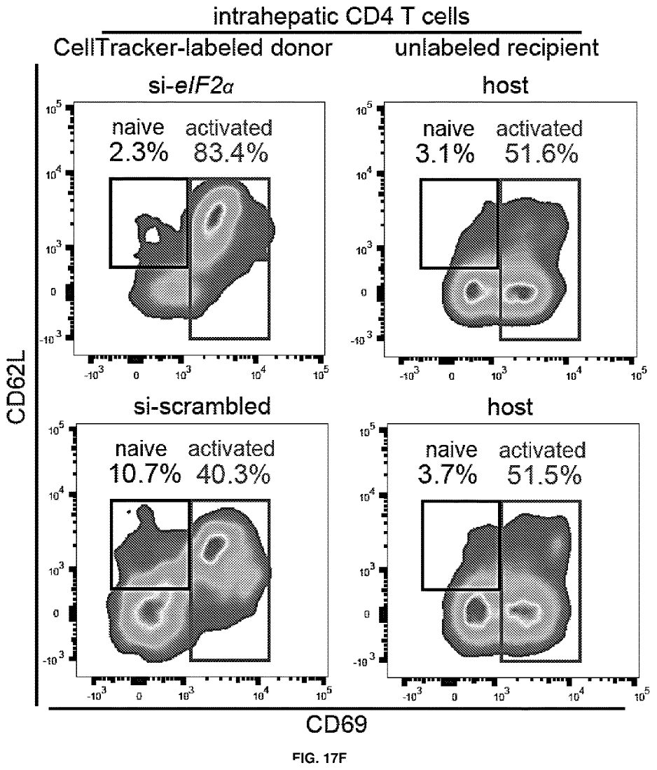

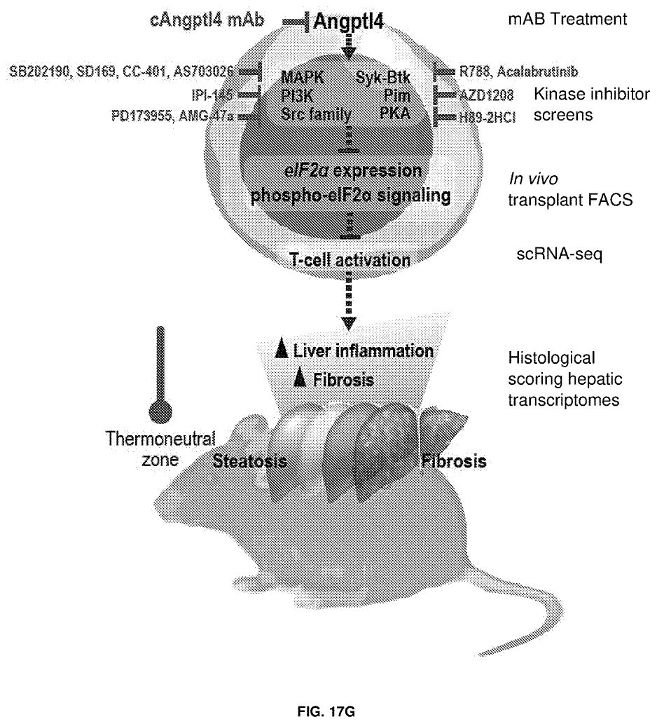

Phosphorylation of eIF2α reduces global translation, allowing cells to conserve resources and to initiate a reconfiguration of gene expression to effectively manage stress conditions. It was sought to decipher how cAngptl4 modifies phospho-eIF2α signalling. Active phospho-eIF2α signalling stimulates the expression of three canonical target genes, activating transcription factor 4 (ATF4), heat shock protein family A member 5 (HSPA5), and DNA damage inducible transcript 3 (DDIT3). As protein kinases are ubiquitously involved in cellular signalling activities, kinase inhibitor screens were conducted to map the regulatory pathways of Angptl4 on eIF2α phosphorylation (based on the readouts from target gene expression) using LPS-activated wild-type and Angptl4_{-/-} CD4₊ T cells. Unsupervised hierarchical clustering of the results revealed 3 clusters of kinase targets corresponding to (i) uninvolved kinases, (ii) Angptl4-independent kinases, and (iii) Angptl4-dependent kinases (FIG. 17A). In wild-type cells, a quarter of the kinase inhibitor has minimal effect on the expression of these target genes compared with DMSO treatment, indicating that these kinases are not involved in phospho-eIF2α-mediated signalling. In DMSO-treated Angptl4_{-/-} cells, target gene expression was already higher than in DMSOtreated wild-type cells, suggesting that Angptl4 deficiency activates phospho-eIF2α-mediated signalling of these target genes. As expected, inhibition of these uninvolved kinases did not alter the target genes compared to DMSO-treated Angptl4_{-/-} cells (FIG. 17A-B).

Twenty-one kinase inhibitors activated the expression of target genes in wild-type cells, indicating that these kinases repress phospho-eIF2α-mediated signalling (FIG. 17A-B). Among them, nine kinase inhibitors against Angptl4-independent kinases derepressed phospho-eIF2α-mediated signalling, increasing the expression of the target genes. These inhibitors further elevated the expression of target genes in Angptl4_{-/-} cells compared with DMSO treatment. The remaining 12 kinase inhibitors did not alter the expression of the target genes in Angptl4_{-/-} cells compared with DMSO-treated Angptl4_{-/-} cells and were identified as Angptl4-dependent (FIG. 17A-B). Angptl4 deficiency did not activate Angptl4-dependent kinases, and futile inhibition by the respective kinase inhibitors yielded minimal changes in the expression of target genes (FIG. 17A-B). Generally, the expression profile of eIF2α followed those of the target genes, revealing a positive correlation between eIF2α and phospho-eIF2α signalling. Angptl4_{-/-} cells had higher eIF2α expression than wild-type cells, which was further increased by LPS (FIG. 17C). Immunoblot analyses showed higher phosphorylated eIF2α in Angptl4_{-/-} CD4 T cells than in wild-type cells, regardless of LPS exposure (FIG. 17D). The results confirmed a suppressive effect of Angptl4 on phospho-eIF2α through the inhibition of eIF2α expression and the kinase inhibitor screens reveal three predominant kinase pathways, i.e., MAPK, PI3K, and Src, in Angptl4-eIF2α axis.

To confirm the in vivo role of phospho-eIF2α signalling in CD4₊ T-cell activation, the effect of eIF2α deficiency on the fate of Angptl4_{-/-} CD4₊ T cells was monitored, which has high endogenous eIF2α, in LIDPAD mice. CellTracker-labelled donor Angptl4_{-/-}CD4₊ T cells treated with si-eIF2α were introduced intravenously into LIDPAD recipient mice (FIG. 17E). As expected, an increase in activated host CD4₊ T cells (unlabelled) was detected in the livers of LIDPAD-fed mice. The deficiency of eIF2α in labelled donor CD4₊ T cells significantly increased the percentage of activated CD4₊ T cells compared to si-scrambled donor CD4₊ T cells (FIG. 17F). Evidently, a reduced eIF2α expression suppresses phospho-eIF2α signalling, leading to more T cell activation. Taken together, it was systematically showed the pivotal role of Angptl4-eIF2α signalling in the activation of T cells that contributes to increased inflammation and fibrosis (FIG. 17G).

### DISCUSSION

In summary, a human relevant diet-induced NAFLD model has been conceived, thus offering opportunities to reliably translate findings in mice to insights into human NAFLD for drug development. Using this model, a domain-specific role for cAngptl4 and phospho-eIF2α signalling has been confirmed in curbing the activation of T-lymphocytes, especially CD4₊ T cells, during fibrosis.

The pathogenesis of NAFLD is a multifactorial and multistep process that involves ill characterized gene-environment interactions and multiorgan involvement (45).

Firstly, it was described that a physiologically relevant refined diet-induced NAFLD model called LIDPAD that recapitulates key transitory stages of human NAFLD with full-range histological and transcriptomic changes. Understanding the network of multisystem interactions requires a physiologically relevant animal model that can mirror the many human NASH features and comorbidities (46). An improved diet inducible liver disease mouse model was established using a refined Liver Disease Progression Aggravation Diet (LIDPAD). Species differences between the LIDPAD model and humans were minimized via thermoneutral mouse housing conditions, while refined diets mitigate batch-to-batch compositional variations, which are metabolic confounders. One key feature of the NAFLD model is its fast disease progression that can capture the full range of anthropometric, physiologic, histological, and transcriptomic anomalies of human pathogenesis in a considerably shorter time frame, i.e., simple steatosis in 1-4 weeks, chronic inflammation in 4-8 weeks, and fibrosis in 12-16 weeks. LIDPAD mice rapidly gain weight and develop impaired glucose homeostasis. Non-invasive longitudinal imaging through magnetic resonance techniques and temporal hepatic transcriptomic profiling pointed to the shift from fatty liver to NASH at week 4 of the diet and the development of fibrosis at 12-16 weeks. Using transcriptomic-guided staging of patients with biopsy-confirmed NASH emphasizes the relevance of LIDPAD mice to model human NAFLD. This model supports a liver-pancreas axis that promotes compensatory pancreatic islet cell hyperplasia, a phenomenon similarly observed in animal models of insulin resistance (47) and in humans, where there is a clear correlation between BMI and β-cell mass (48) and thereafter fatty liver and the development of type-2 diabetes (49). Furthermore, LIDPAD mice also displayed NAFLD-associated vasculopathy, with heightened endothelial expression of CXCL12 in the aortas and the liver vasculature (50). These various metabolic dysfunctions and extrahepatic comorbidities are collectively absent in current NAFLD models.

Secondly, using the LIDPAD model, the intrahepatic immune landscape during NASH was delineated at single-cell resolution, which revealed the importance of adaptive immunity in disease progression.

A single-cell analysis of the intrahepatic immune landscape of a human relevant diet-induced NAFLD mouse model was performed to reveal an indispensable role of adaptive immune cell remodelling in disease aggravation characterized by increased activated T-lymphocytes. The activation of CD4₊ T cells was shown to depend on eIF2α-mediated eukaryotic translation initiation during fibrosis.

Understanding the time frame for the fatty liver to NASH transition in the LIDPAD model allowed for in-depth examination of the role of immune cells in disease aggravation to fibrosis. Histological examination revealed elevated infiltration of cells from the adaptive immune system at 8 weeks of LIDPAD feeding, which persisted in a more advanced stage. The single-cell surveillance of intrahepatic immune cells of livers in LIDPAD mice at this transition revealed an overrepresentation of adaptive immune cells, including B- and T-lymphocytes. These abnormal immune responses are predominantly orchestrated by CD4₊ helper T cells, which are highly activated throughout the early and advanced NASH stages in the LIDPAD model. This corroborates findings that T-lymphocytes are heavily involved in NAFLD deterioration (50-52). A recent study also identified an integral role of activated CD4₊ T cells at fibrotic sites in NASH-driven hepatic inflammation and fibrosis (53). Mechanistically, the results disclosed here revealed that Angptl4-eIF2α signalling is pivotal for the activation of CD4₊ T cells. CD4₊ T cells also mediate NASH exacerbation by modulating the activation of CD8₊ T cells, NKT cells and fibrogenic HSCs. Mechanistically, CD8₊ cytotoxic T cells drive the NAFL-NASH transition through elevated proinflammatory cytokine secretion and auto aggressive killing of hepatocytes (54). Different CD4₊ T cell subsets exhibit diverse effects on liver fibrosis. Th2- and Th17-derived cytokines stimulate collagen production and TGFβR-dependent hepatic fibrosis (55). Notably, NAFLD patients also exhibit increased serum proinflammatory and profibrogenic cytokine signatures coupled with increased activated CD4₊ T cells (55). Collectively, the multifaceted role of the CD4₊ T-cell response remains an integral driver in NASH exacerbation.

Finally, it was demonstrated that cANGPTL4 orchestrates the remodelling of hyperactive adaptive immunity, which aggravates liver inflammation and fibrosis, and shows that its inhibition is a promising approach to ameliorating NASH.

ANGPLT4-eIF2α signalling pathway was further deciphered and found essential for the activation of CD4₊ T cells (FIG. 17G). The immunoneutralization of cANGPTL4 effectively diminishes the NASH-promoting immunological response and delays liver fibrosis.

These results particularly focused on the exacerbation of NASH with fibrosis and affirmed an immunomodulatory role for cANGPTL4 in the intrahepatic adaptive immune response through the ANGPTL4-eIF2α axis. Very little is known about the role active phospho-eIF2α signalling in the immunopathology of NAFLD. It was shown that active eIF2α supports the maturation and clonal expansion of CD4₊ T cells, which subsequently mediate the activation of other lymphocytes to create an inflamed and fibrogenic liver environment in NASH. Indeed, inhibition of the components in the translation initiation complex abrogates CD4₊ T-cell activation and differentiation, which was observed in the NAFLD mice bearing eIF2α-knockdown CD4₊ T cells via adoptive transfer. These results also showed reduced fibrosis in myeloid-specific Angptl4-knockout mice, likely due to reduced T-cell infiltration rather than their activation status. The immunomodulation of T cells offers an exciting avenue for alleviating chronic inflammation that contributes to NASH. It has been shown that immunotherapy with the administration of mAb against cANGPTL4 effectively diminishes T-lymphocyte activation and delays liver fibrosis in NASH via modulation of phosphorylated-eIF2α signalling.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The methods, and uses described herein are presently representative of preferred embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The content of all documents and patent documents cited herein is incorporated by reference in their entirety.

### REFERENCES

1. F. Kanwal, et al, Preparing for the NASH epidemic: a call to action. Metabolism 122, 154822 (2021).
2. Z. M. Younossi, et al, Global epidemiology of non-alcoholic fatty liver disease-Meta-analytic assessment of prevalence, incidence, and outcomes. Hepatology 64, 73-84 (2016).
3. K. M. Flegal, et al., Ogden, Prevalence of obesity and trends in the distribution of body mass index among US adults, 1999-2010. JAMA 307, 491-497 (2012).
4. M. Romero-Gomez, S. Zelber-Sagi, M. Trenell, Treatment of NAFLD with diet, physical activity and exercise. J Hepatol 67, 829-846 (2017).
5. A. A. Sohrabpour, M. Mohamadnejad, R. Malekzadeh, Review article: the reversibility of cirrhosis. Aliment Pharmacol Ther 36, 824-832 (2012).
6. C. H. Ng, et al., Mortality Outcomes by Fibrosis Stage in Nonalcoholic Fatty Liver Disease: A Systematic Review and Meta-analysis. Clin Gastroenterol Hepatol, (2022).
7. S. Bruzzi, et al., B2-Lymphocyte responses to oxidative stress-derived antigens contribute to the evolution of nonalcoholic fatty liver disease (NAFLD). Free Radic Biol Med 124, 249-259 (2018).
8. S. Sutti, E. Albano, Adaptive immunity: an emerging player in the progression of NAFLD. Nat Rev Gastroenterol Hepatol 17, 81-92 (2020).
9. M. Ghazarian, et al, Type I Interferon Responses Drive Intrahepatic T cells to Promote Metabolic Syndrome. Sci Immunol 2, (2017).
10. S. Raza, S. Rajak, A. Upadhyay, A. Tewari, R. Anthony Sinha, Current treatment paradigms and emerging therapies for NAFLD/NASH. Front Biosci (Landmark Ed) 26, 206-237 (2021).
11. Eslarn M. et al. A new definition for metabolic dysfunction-associated fatty liver disease: An international expert consensus statement. J. Hepatol. 2020; 73:202-209,
12. P. Zhu, et al, Angiopoietin-like 4: a decade of research. Biosci Rep 32, 211-219 (2012).
13. X. Lei, F. Shi, D. Basu, A. Huq, S. Routhier, R. Day, W. Jin, Proteolytic Processing of Angiopoietin like Protein 4 by Proprotein Convertases Modulates Its Inhibitory Effects on Lipoprotein Lipase Activity *. Journal of Biological Chemistry 286, 15747-15756 (2011).
14. Y. Y. Goh, et al, Angiopoietin-like 4 interacts with matrix proteins to modulate wound healing. J Biol Chem 285, 32999-33009 (2010).
15. L. Li, et al, Antibody Treatment against Angiopoietin-Like 4 Reduces Pulmonary Edema and Injury in Secondary Pneumococcal Pneumonia. mBio 10, (2019).
16. K. H. Jung, et al., ANGPTL4 exacerbates pancreatitis by augmenting acinar cell injury through upregulation of C5a. EMBO Mol Med 12, e11222 (2020).
17. C. Hubers, et al, Primary tumor-derived systemic nANGPTL4 inhibits metastasis. J Exp Med 220, (2023).
18. R. L. Huang, et al, ANGPTL4 modulates vascular junction integrity by integrin signaling and disruption of intercellular VE-cadherin and claudin-5 clusters. Blood 118, 3990-4002 (2011).
19. T. Phua, et al, Angiopoietin-like 4 Mediates Colonic Inflammation by Regulating Chemokine Transcript Stability via Tristetraprolin. Sci Rep 7, 44351 (2017).
20. W. K. J. Wee, et al, Single-cell analysis of skin immune cells reveals an Angptl4-ifi20b axis that regulates monocyte differentiation during wound healing. Cell Death Dis 13, 180 (2022).
21. A. D. Yates, et al, Ensembl 2020. Nucleic Acids Res 48, D682-D688 (2020).
22. D. Kim, B. Langmead, S. L. Salzberg, HISAT: a fast spliced aligner with low memory requirements. Nat Methods 12, 357-360 (2015).
23. Y. Liao, G. K. Smyth, W. Shi, featureCounts: an efficient general purpose program for assigning sequence reads to genomic features. Bioinformatics 30, 923-930 (2014).
24. M. I. Love, W. Huber, S. Anders, Moderated estimation of fold change and dispersion for RNA seq data with DESeq2. Genome Biol 15, 550 (2014).
25. X. Xiong, et al, Landscape of Intercellular Crosstalk in Healthy and NASH Liver Revealed by Single-Cell Secretome Gene Analysis. Mol Cell 75, 644-660 e645 (2019).
26. V. Matyash, G. Liebisch, T. V. Kurzchalia, A. Shevchenko, D. Schwudke, Lipid extraction by methyl-tert-butyl ether for high-throughput lipidomics. J Lipid Res 49, 1137-1146 (2008).
27. J. G. McDonald, D. D. Smith, A. R. Stiles, D. W. Russell, A comprehensive method for extraction and quantitative analysis of sterols and secosteroids from human plasma. J Lipid Res 53, 1399-1409 (2012).
28. Y. Hao, et al, Integrated analysis of multimodal single-cell data. Cell 184, 3573-3587 e3529 (2021).
29. M. V. Kuleshov, et al, a comprehensive gene set enrichment analysis web server 2016 update. Nucleic Acids Res 44, W90-97 (2016).
30. K. Street, et al, Slingshot: cell lineage and pseudotime inference for single-cell transcriptomics. BMC Genomics 19, 477 (2018).
31. K. Van den Berge, et al, Trajectory-based differential expression analysis for single-cell sequencing data. Nat Commun 11, 1201 (2020).
32. D. Aran, et al, Reference-based analysis of lung single-cell sequencing reveals a transitional profibrotic macrophage. Nat Immunol 20, 163-172 (2019).
33. T. S. Heng, M. W. Painter, C. Immunological Genome Project, The Immunological Genome Project: networks of gene expression in immune cells. Nat Immunol 9, 1091-1094 (2008).
34. T. S. P. Heng, et al. The Immunological Genome Project, The Immunological Genome Project: networks of gene expression in immune cells. Nature Immunology 9, 1091-1094 (2008).
35. Z. A. Clarke, et al, Tutorial: guidelines for annotating single-cell transcriptomic maps using automated and manual methods. Nature Protocols 16, 2749-2764 (2021).
36. H. Shen, C. Gu, T. Liang, H. Liu, F. Guo, X. Liu, Unveiling the heterogeneity of NKT cells in the liver through single cell RNA sequencing. Sci Rep 10, 19453 (2020).
37. R. D. Lee, S. A. Munro, T. P. Knutson, R. S. LaRue, L. M. Heltemes-Harris, M. A. Farrar, Singlecell analysis identifies dynamic gene expression networks that govern B cell development and transformation. Nat Commun 12, 6843 (2021).
38. A. Remmerie, et al, Osteopontin Expression Identifies a Subset of Recruited Macrophages Distinct from Kupffer Cells in the Fatty Liver. Immunity 53, 641-657 e614 (2020).
39. C. Bleriot, F. Ginhoux, Understanding the Heterogeneity of Resident Liver Macrophages. Front Immunol 10, 2694 (2019).
40. C. C. Brown, et al, Transcriptional Basis of Mouse and Human Dendritic Cell Heterogeneity. Cell 179, 846-863 e824 (2019).
41. A. Musumeci, K. Lutz, E. Winheim, A. B. Krug, What Makes a pDC: Recent Advances in Understanding Plasmacytoid DC Development and Heterogeneity. Front Immunol 10, 1222 (2019).
42. G. Cholankeril, R. Patel, S. Khurana, S. K. Satapathy, Hepatocellular carcinoma in non alcoholic steatohepatitis: Current knowledge and implications for management. World J Hepatol 9, 533-543 (2017).
43. V. Braunersreuther, G. L. Viviani, F. Mach, F. Montecucco, Role of cytokines and chemokines in non-alcoholic fatty liver disease. World J Gastroenterol 18, 727-735 (2012).
44. M. Ryaboshapkina, M. Hammar, Human hepatic gene expression signature of non-alcoholic fatty liver disease progression, a meta-analysis. Sci Rep 7, 12361 (2017).
45. C. D. Byrne, G. Targher, NAFLD: a multisystem disease. J Hepatol 62, S47-64 (2015).
46. D. Chua, Z. S. Low, G. X. Cheam, A. S. Ng, N. S. Tan, Utility of Human Relevant Preclinical Animal Models in Navigating NAFLD to MAFLD Paradigm. International Journal of Molecular Sciences 23, 14762 (2022).
47. O. Escribano, et al, Beta-Cell hyperplasia induced by hepatic insulin resistance: role of a liver-pancreas endocrine axis through insulin receptor A isoform. Diabetes 58, 820-828 (2009).
48. Y. Saisho, A. E. Butler, E. Manesso, D. Elashoff, R. A. Rizza, P. C. Butler, beta-cell mass and turnover in humans: effects of obesity and aging. Diabetes Care 36, 111-117 (2013).
49. Z. M. Younossi, et al, The global epidemiology of NAFLD and NASH in patients with type 2 diabetes: A systematic review and meta-analysis. J Hepatol 71, 793-801 (2019).
50. C. Y. Ng, et al, Endothelial-immune crosstalk contributes to vasculopathy in nonalcoholic fatty liver disease. EMBO Rep 23, e54271 (2022).
51. P. Hirsova, A. O. Bamidele, H. Wang, D. Povero, X. S. Revelo, Emerging Roles of T Cells in the Pathogenesis of Nonalcoholic Steatohepatitis and Hepatocellular Carcinoma. Front Endocrinol (Lausanne) 12, 760860 (2021).
52. J. T. Haas et al, Transcriptional Network Analysis Implicates Altered Hepatic Immune Function in NASH development and resolution. Nat Metab 1, 604-614 (2019).
53. Z. Her, et al, CD4+ T Cells Mediate the Development of Liver Fibrosis in High Fat Diet-Induced NAFLD in Humanized Mice. Frontiers in Immunology 11, (2020).
54. M. Dudek, et al, Auto-aggressive CXCR6(+) CD8 T cells cause liver immune pathology in NASH. Nature 592, 444-449 (2021).
55. Y. Zhou, H. Zhang, Y. Yao, X. Zhang, Y. Guan, F. Zheng, CD4+ T cell activation and inflammation in NASH-related fibrosis. Frontiers in immunology 13, (2022).
56. L Li et al, Angiopoietin-like 4 Increases Pulmonary Tissue Leakiness and Damage during Influenza Pneumonia. Cell Reports. Volume 10, Issue 5, 10 Feb. 2015, Pages 654-663.
57. W Wee et al, Single-cell analysis of skin immune cells reveals an Angptl4-ifi20b axis that regulates monocyte differentiation during wound healing. Cell Death & Disease volume 13, Article number: 180 (2022).
58. S. Goa, X Zheng, J Wu, A graphene oxide-based aptasensor for ANGPTL4 biomarker detection. Sensors and Actuators B: Chemical. Volume 345, 15 Oct. 2021, 130389 (2021).

1. A method for treating, ameliorating, preventing or delaying liver fibrosis or a disease associated with liver fibrosis in a subject or liver inflammation in a subject suffering from liver fibrosis, the method comprising administering to the subject an effective amount of an angiopoietin-like 4 protein (ANGPTL4) antagonist.

2. The method of claim 1, wherein the disease associated with liver fibrosis is selected from non-alcoholic steatohepatitis (NASH), autoimmune hepatitis, congenital liver fibrosis, non-alcoholic fatty liver disease (NAFLD), cholestatic liver disease, alcoholic hepatitis, and viral hepatitis.

3. The method of claim 1, wherein the disease associated with liver fibrosis is non-alcoholic steatohepatitis (NASH) or wherein the liver fibrosis is associated with non-alcoholic steatohepatitis (NASH).

4. The method of claim 1, wherein the disease associated with liver fibrosis is non-alcoholic fatty liver disease (NAFLD) or wherein the liver fibrosis is associated with non-alcoholic fatty liver disease (NAFLD).

5. The method of claim 1, wherein the ANGPTL4 antagonist is an antisense molecule; or an aptamer; or an siRNA molecule; or an anti-ANGPTL4 antibody.

6. The method of claim 1, wherein the ANGPTL4 antagonist is directed against the C-terminal fibrinogen-like domain of angiopoietin like 4 (cANGPTL4).

7. The method of claim 1, wherein the ANGPTL4 antagonist neutralizes, blocks, inhibits, reduces or interferes with the expression and/or activity of the C-terminal fibrinogen-like domain of angiopoietin-like 4 (cANGPTL4).

8. The method of claim 1, wherein the ANGPTL4 antagonist is an anti-cANGPTL4 antibody, preferably a monoclonal antibody, more preferably a neutralizing monoclonal antibody.

9. The method of claim 8, wherein the anti-cANGPTL4 antibody is a monoclonal antibody that binds to the same epitope as mAb 11F6C4.

10. The method of claim 8, wherein the anti-cANGPTL4 antibody is a humanized antibody, preferably a humanized mAb 11F6C4.

11. The method of claim 8, wherein the anti-cANGPTL4 antibody specifically binds to the C-terminal fibrinogen-like domain of ANGPTL4.

12. The method of claim 1, wherein the ANGPTL4 antagonist is a siRNA molecule, preferably a ANGPTL4-siRNA molecule.

13. The method of claim 12, wherein the siRNA molecule targets a mRNA sequence encoding ANGPTL4.

14. The method of claim 1, wherein the subject is a human.

15. (canceled)

16. A method for regulating an immune response in a subject suffering from liver fibrosis or a disease associated with liver fibrosis, the method comprising administering an effective amount of an angiopoietin-like 4 protein (ANGPTL4) antagonist, wherein the administration induces the number, activity and/or effector functions of a population of regulatory T cells or activated T-cells, preferably CD8⁺ and/or CD4+T cells, to be reduced in the liver of the subject.

17. An in vitro method of reducing fibrosis or liver inflammation in a liver sample, comprising: contacting the liver sample with an angiopoietin-like 4 protein (ANGPTL4) antagonist.

18. (canceled)

19. (canceled)

20. The method of claim 16, wherein the ANGPTL4 antagonist is directed against the C-terminal fibrinogen-like domain of angiopoietin like 4 (cANGPTL4).

21. The method of claim 17, wherein the ANGPTL4 antagonist is directed against the C-terminal fibrinogen-like domain of angiopoietin like 4 (cANGPTL4).


SEQUENCE LISTING (20 sequences)

SEQ ID NO: 1 - protein, 406 aa, Homo sapiens
MSGAPTAGAALMLCAATAVLLSAQGGPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 2 - protein, 223 aa, Homo sapiens
SRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMA

SEQ ID NO: 3 - DNA, 1872 bp, Homo sapiens
  source 1..1872: mol_type = other DNA
agaagccgagctgagcggatcctcacacgactgtgatccgattctttccagcggcttctgcaaccaagcgggtcttacccccggtcctccgcgtctccagtcctcgcacctggaaccccaacgtccccgagagtccccgaatccccgctcccaggctacctaagaggatgagcggtgctccgacggccggggcagccctgatgctctgcgccgccaccgccgtgctactgagcgctcagggcggacccgtgcagtccaagtcgccgcgctttgcgtcctgggacgagatgaatgtcctggcgcacggactcctgcagctcggccaggggctgcgcgaacacgcggagcgcacccgcagtcagctgagcgcgctggagcggcgcctgagcgcgtgcgggtccgcctgtcagggaaccgaggggtccaccgacctcccgttagcccctgagagccgggtggaccctgaggtccttcacagcctgcagacacaactcaaggctcagaacagcaggatccagcaactcttccacaaggtggcccagcagcagcggcacctggagaagcagcacctgcgaattcagcatctgcaaagccagtttggcctcctggaccacaagcacctagaccatgaggtggccaagcctgcccgaagaaagaggctgcccgagatggcccagccagttgacccggctcacaatgtcagccgcctgcaccggctgcccagggattgccaggagctgttccaggttggggagaggcagagtggactatttgaaatccagcctcaggggtctccgccatttttggtgaactgcaagatgacctcagatggaggctggacagtaattcagaggcgccacgatggctcagtggacttcaaccggccctgggaagcctacaaggcggggtttggggatccccacggcgagttctggctgggtctggagaaggtgcatagcatcacgggggaccgcaacagccgcctggccgtgcagctgcgggactgggatggcaacgccgagttgctgcagttctccgtgcacctgggtggcgaggacacggcctatagcctgcagctcactgcacccgtggccggccagctgggcgccaccaccgtcccacccagcggcctctccgtacccttctccacttgggaccaggatcacgacctccgcagggacaagaactgcgccaagagcctctctggaggctggtggtttggcacctgcagccattccaacctcaacggccagtacttccgctccatcccacagcagcggcagaagcttaagaagggaatcttctggaagacctggcggggccgctactacccgctgcaggccaccaccatgttgatccagcccatggcagcagaggcagcctcctagcgtcctggctgggcctggtcccaggcccacgaaagacggtgactcttggctctgcccgaggatgtggccgttccctgcctgggcaggggctccaaggaggggccatctggaaacttgtggacagagaagaagaccacgactggagaagccccctttctgagtgcaggggggctgcatgcgttgcctcctgagatcgaggctgcaggatatgctcagactctagaggcgtggaccaaggggcatggagcttcactccttgctggccagggagttggggactcagagggaccacttggggccagccagactggcctcaatggcggactcagtcacattgactgacggggaccagggcttgtgtgggtcgagagcgccctcatggtgctggtgctgttgtgtgtaggtcccctggggacacaagcaggcgccaatggtatctgggcggagctcacagagttcttggaataaaagcaacctcagaaca

SEQ ID NO: 4 - protein, 410 aa, Mus musculus
MRCAPTAGAALVLCAATAGLLSAQGRPAQPEPPRFASWDEMNLLAHGLLQLGHGLREHVERTRGQLGALERRMAACGNACQGPKGKDAPFKDSEDRVPEGQTPETLQSLQTQLKAQNSKIQQLFQKVAQQQRYLSKQNLRIQNLQSQIDLLAPTHLDNGVDKTSRGKRLPKMTQLIGLTPNATHLHRPPRDCQELFQEGERHSGLFQIQPLGSPPFLVNCEMTSDGGWTVIQRRLNGSVDFNQSWEAYKDGFGDPQGEFWLGLEKMHSITGNRGSQLAVQLQDWDGNAKLLQFPIHLGGEDTAYSLQLTEPTANELGATNVSPNGLSLPFSTWDQDHDLRGDLNCAKSLSGGWWFGTCSHSNLNGQYFHSIPRQRQERKKGIFWKTWKGRYYPLQATTLLIQPMEATAAS

SEQ ID NO: 5 - protein, 223 aa, Mus musculus
THLHRPPRDCQELFQEGERHSGLFQIQPLGSPPFLVNCEMTSDGGWTVIQRRLNGSVDFNQSWEAYKDGFGDPQGEFWLGLEKMHSITGNRGSQLAVQLQDWDGNAKLLQFPIHLGGEDTAYSLQLTEPTANELGATNVSPNGLSLPFSTWDQDHDLRGDLNCAKSLSGGWWFGTCSHSNLNGQYFHSIPRQRQERKKGIFWKTWKGRYYPLQATTLLIQPME

SEQ ID NO: 6 - DNA, 1916 bp, Homo sapiens
  source 1..1916: mol_type = other DNA
gctttataaagtggggctttaggtgcaaccgtgaaacgcttatgagctacgggctccagatcttcttctgcaccagagcaagtctaagtctgagccggctcccccagaactccagctgctgggtcttgaactcctgcgttccggagtcctagcgttgctgcacccaaggccacccccagaatcatgcgctgcgctccgacagcaggcgctgccctggtgctatgcgcggctactgcggggcttttgagcgcgcaagggcgccctgcacagccagagccaccgcgctttgcatcctgggacgagatgaacttgctggctcacgggctgctacagctcggccatgggctgcgcgaacacgtggagcgcacccgtgggcagctgggcgcgctggagcgccgcatggctgcctgtggtaacgcttgtcaggggcccaagggaaaagatgcacccttcaaagactccgaggatagagtccctgaaggccagactcctgagactctgcagagtttgcagactcagctcaaggctcaaaacagcaagatccagcaattgttccagaaggtggcccagcagcagagatacctatcaaagcagaatctgagaatacagaatcttcagagccagatagacctcttggcccccacgcacctagacaatggagtagacaagacttcgaggggaaagaggcttcccaagatgacccagctcattggcttgactcccaacgccacccacttacacaggccgccccgggactgccaggaactcttccaagaaggggagcggcacagtggacttttccagatccagcctctggggtctccaccatttttggtcaactgtgagatgacttcagatggaggctggacagtgattcagagacgcctgaacggctctgtggacttcaaccagtcctgggaagcctacaaggatggcttcggagatccccaaggcgagttctggctgggcctggaaaagatgcacagcatcacagggaaccgaggaagccaattggctgtgcagctccaggactgggatggcaatgccaaattgctccaatttcccatccatttggggggtgaggacacagcctacagcctgcagctcactgagcccacggccaatgagctgggtgccaccaatgtttcccccaatggcctttccctgcccttctctacttgggaccaagaccatgacctccgtggggaccttaactgtgccaagagcctctctggtggctggtggtttggtacctgtagccattccaatctcaatggacaatacttccactctatcccacggcaacggcaggagcgtaaaaagggtatcttctggaaaacatggaagggccgctactatcctctgcaggctaccaccctgctgatccagcccatggaggctacagcagcctcttagcctcctcactggagcctggttccaggcctaagaagacagtgactttggttgtggccctgagatttggccattctctgctgggggcaggagctctaagtagggctatctgcgtcttgtggacaaagaagaagcccgtaactggagagactggaggaccccttttccgtgttggggtctgcaagcattgttgtctgaaacagtcagagcaacaggaaacaaatggcccagatccagaaaacatgggctcgaggggcactgaatatcacttctcgcctaccagagaagttggggatgcagagggaccactacagtccaactagctgggcccttaatggcggactcagtcatattgactgactggagacagggtgccaggagccctggatacactcatggtgctgttgtaggtgctgtggatgcacaggtgctaactgtggttcccaggcacagctcacagcattcttacaataaaaacaacctcagaacaaaaaaaaaaaaaa

SEQ ID NO: 7 - protein, 119 aa, synthetic construct
QITLKESGPTLVKPTQTLTLTCTFSGFSLSTSGMGVGWIRQPPGKALEWLAHIWWDDDKYYNPSLKSRLTITKDTSKNQVVLTMTNMDPVDTATYYCARKDYGSSYDYWGQGTLVTVSS

SEQ ID NO: 8 - protein, 111 aa, synthetic construct
DIVMTQSPDSLAVSLGERATINCKASQSVDYDGDSYLNWYQQKPGQPPKLLIYTASNLESGVPDRFSGSGSGTDFTLTISSLQAEDVAVYYCQQSNEDPWTFGQGTKVEIK

SEQ ID NO: 9 - protein, 119 aa, synthetic construct
QVTLKESGPVLVKPTETLTLTCTVSGFSLSTSGMGVGWIRQPPGKALEWLAHIWWDDDKYYNPSLKSRLTISKDTSKSQVVLTMTNMDPVDTATYYCARKDYGSSYDYWGQGTLVTVSS

SEQ ID NO: 10 - protein, 111 aa, synthetic construct
EIVLTQSPATLSLSPGERATLSCKASQSVDYDGDSYLNWYQQKPGQAPRLLIYTASNLESGIPARFSGSGSGTDFTLTISSLEPEDFAVYYCQQSNEDPWTFGQGTKLEIK

SEQ ID NO: 11 - protein, 119 aa, synthetic construct
QVTLKESGPTLVKPTQTLTLTCTFSGFSLSTSGMGVGWIRQPPGKALEWLAHIWWDDDKYYNPSLKSRLTISKDTSKNQVVLTMTNMDPVDTATYYCARKDYGSSYDYWGQGTTVTVSS

SEQ ID NO: 12 - protein, 111 aa, synthetic construct
DIVMTQSPLSLPVTPGEPASISCKASQSVDYDGDSYLNWYLQKPGQSPRLLIYTASNLESGVPDRFSGSGSGTDFTLKISRVEAEDVGVYYCFQSNEDPWTFGQGTKLEIK

SEQ ID NO: 13 - protein, 119 aa, synthetic construct
QITLKESGPTLVKPTQTLTLTCTFSGFSLSTSGMGVGWIRQPPGKALEWLAHIWWDDDKYYNPSLKSRLTITKDTSKNQVDLTMTFMDPWDTATYYCAHKDYGSSYDYWGQGTLVTVSS

SEQ ID NO: 14 - protein, 111 aa, synthetic construct
DIQMTQSPSSLSASVGDRVTITCKASQSVDYDGDSYLNWYQQKPGKAPNLLIYTASNLESGVPSRFSGSGSGTDFTLTISSLQPEDFATYYCQQSNEDPWTFGEGTKVEIK

SEQ ID NO: 15 - protein, 118 aa, synthetic construct
QVQLQESGPGILKPSQTLSLTCSFSGFSLSTSGMGVGWIRQPSGKGLERLAHIWWDDDKYYNPSLKSQLTISKDTSRNQVFLKIISVDTADTATYYCARKDYGSSYDYRGQGTTVTVS

SEQ ID NO: 16 - protein, 112 aa, synthetic construct
DIELTQSPASLAVSLGQRATISCKASQSVDYDGDSYLNRFQQKPGQPPKLLIYTASNLESGIPARFSGSGSGTDFTLNIHPVEEEDAATYYCQQSNEDPWTFGGGTKLEIKR

SEQ ID NO: 17 - RNA, 31 nt, synthetic construct
  source 1..31: mol_type = other RNA
aaagctgcaagatgacctcagatggaggctg

SEQ ID NO: 18 - RNA, 31 nt, synthetic construct
  source 1..31: mol_type = other RNA
taaacagcctccatctgaggtcatcttgcag

SEQ ID NO: 19 - RNA, 81 nt, synthetic construct
  source 1..81: mol_type = other RNA
tcgaggcagcacctgcgaattcagcatctgcattcaagagatgcagatgctgaattcgcaggtgctgcttttttacgcgta

SEQ ID NO: 20 - RNA, 80 nt, synthetic construct
  source 1..80: mol_type = other RNA
agcttacgcgtaaaaagcagcacctgcgaattcagcatctgcatctcttgaatgcagatgctgaattcgcaggtgctgcc